(19) United States
(12) Patent Application Publication
(10) Pub. No.: US 2017/0014455 A1
(43) Pub. Date: Jan. 19, 2017

(54) INDUCING BROWN FAT FATE AND FUNCTION

(71) Applicant: The J. David Gladstone Instiutes, a testamentary trust established under the Will of J. David Glads, San Francisco, CA (US)

(72) Inventors: Baoming Nie, San Francisco, CA (US); Sheng Ding, Orinda, CA (US)

(21) Appl. No.: 15/124,410

(22) PCT Filed: Mar. 12, 2015
(86) PCT No.: PCT/US2015/020108

§ 371 Date: Sep. 8, 2016

Related U.S. Application Data
(60) Provisional application No. 61/952,216, filed on Mar. 13, 2014.

(51) Int. Cl. A61K 35/35; C12N 5/077; A61K 31/4704; A61K 31/192; A61K 31/4418

(52) U.S. Cl. CPC A61K 35/35; A61K 31/192; A61K 31/4418; A61K 31/4704; C12N 5/0653; C12N 2501/06; C12N 2501/999; C12N 2501/385

(57) ABSTRACT

Methods and compositions are described herein for generating brown adipose cells and tissues that involve contacting one or more starting cells with bexarotene, ciclopirox, IOX2, or combinations thereof. When administered in vivo, subjects receiving bexarotene, ciclopirox, IOX2, or combinations thereof have reduced white adipose tissue mass (with enhanced beige features) as well as enlarged brown fat tissue compared to a control mammal that did not receive the bexarotene, ciclopirox, IOX2, or combinations thereof. The subjects also have increased energy expenditure, generate more heat, and/or consume more oxygen, than a control mammal that did not receive the bexarotene, ciclopirox, IOX2, or combinations thereof.

### CROSS REFERENCE TO RELATED APPLICATIONS

This application claims benefit of priority to the filing date of U.S. Provisional Application Ser. No. 61/952,216, filed Mar. 13, 2014, the contents of which are specifically incorporated herein by reference in their entity.

### BACKGROUND

Obesity represents the most prevalent of body weight disorders, and it is the most important nutritional disorder in the Western world, with estimates of its prevalence ranging from 30% to 50% of the middle-aged population. The number of overweight and obese Americans has continued to increase since 1960, a trend that is not slowing down. Today, 64.5 percent of adult Americans (about 127 million) are categorized as being overweight or obese. Obesity is becoming a growing concern as the number of people with obesity continues to increase and more is learned about the negative health effects of obesity. Each year, obesity causes at least 300,000 deaths in the U.S., and healthcare costs of American adults with obesity amount to more than $125 billion (American Obesity Association). Severe obesity, in which a person is 100 pounds or more over ideal body weight, in particular poses significant risks for severe health problems. Accordingly, a great deal of attention is being focused on treating patients with obesity.

Even mild obesity increases the risk for premature death, diabetes, hypertension, atherosclerosis, gallbladder disease and certain types of cancer. Because of its high prevalence and significant health consequences, its treatment should be a high public health priority. Therefore, a better understanding of the mechanism for weight loss is needed, as well as better methods and therapeutics for treating obesity and inducing weight loss.

While obesity is a known risk factor for metabolic diseases (e.g. diabetes), visceral white fat (i.e. visceral white adipose tissue or visceral WAT) is particularly associated with disease risk (Klein, S., et al. Diabetes Care 30: 1647-1652 (2007)). In contrast, brown adipose tissue (BAT) can be beneficial to a subject.

### SUMMARY

Methods and compositions are described herein for generating one or more brown adipose cells from one or more starting cells that involves contacting one or more of the starting cells with bexarotene, ciclopirox, IOX2, or combinations thereof, to thereby generate one or more brown adipose cells.

As described herein, subjects receiving bexarotene, ciclopirox, IOX2, or combinations thereof, have lower body fat, reduced white adipose tissue mass, increased energy expenditure, generate more heat, and/or consume more oxygen, than a control mammal that did not receive the bexarotene, ciclopirox, IOX2, or combinations thereof.

Methods and compositions are therefore also described herein for administering bexarotene, ciclopirox, IOX2, or combinations thereof to a mammal, to thereby reduce body fat, reduce white adipose tissue mass, increase energy expenditure, generate more heat, and/or consume more oxygen, than a control mammal that did not receive the bexarotene, ciclopirox, IOX2, or combinations thereof.

### DESCRIPTION OF THE FIGURES

FIG. 1A-1G illustrate that bexarotene induces adipogenesis in C2C12 and C3H10T1/2 cells, switching adipocytes to a brown adipose cell type. FIG. 1A illustrates the high-throughput screening (HTS) strategy and the timing in days (D-1, D-2, D0, etc.) for the steps employed in identifying compounds that modulate adipogenesis in C2C12 myoblast cells. The assays were performed in 384-well plates. FIG. 1B shows images of C2C12 myoblast cells after staining with Oil-red-O (ORO), illustrating adipogenesis induced by bexarotene during the high-throughput screen. Images of cells treated with retinoic acid (RA), 9-cis-retinoic acid (9-cis-RA), 1,25-dihydroxyvitamin D3 (1,25(OH)₂D₃), and reversine (2-(4-morpholinoanilino)-6-cyclohexylaminopurine) after staining with ORO are also shown. FIG. 1C illustrates ORO staining in C2C12 myoblast cells showing adipogenesis after treatment with bexarotene, rosiglitazone and bexarotene+rosiglitazone compared to an untreated Control (Ctrl). FIG. 1D illustrates ORO staining of C3H10T1/2 mesenchymal cells showing adipogenesis by bexarotene. FIG. 1E shows Ucp1 and Pgc1α expression levels in the C3H10T1/2 mesenchymal cells shown in FIG. 1D, as detected by real-time PCR. Expression of the thermogenic genes Ucp1 and Pgc1α in the presence of bexarotene (Bex10) with forskolin (Fsk) stimulation was greater than when the COX-2 agonist cPGI2 was present, even with no forskolin stimulation. Data were normalized to Control (Ctrl) and represent mean±SEM, **p<0.01, ***p<0.001. FIG. 1F illustrates ORO staining in primary mouse embryonic fibroblasts showing adipogenesis in the presence of bexarotene and rosiglitazone. FIG. 1G graphically illustrates Ucp1 and PPARγ expression in the primary mouse embryonic fibroblasts shown in FIG. 1F, as detected by real-time PCR. Data were normalized to those of mouse embryonic fibroblasts and represent mean±SEM, ***p<0.001.

FIG. 2A-2I show that bexarotene induces adipogenesis in C2C12 cells and switches adipocytes to a ‘browning’ phenotype. C2C12 cells (FIG. 2A-2C), 3T3-L1 cells (FIG. 2D-2F), and pre-brown adipose tissue cells (FIG. 2G-2I) were treated with or without 1 μM bexarotene (Bex1) or 10 μM bexarotene (Bex10) in adipogenesis medium for the first 2 days. Then cells were stained with Oil-red-O (ORO) on Day 6 as shown in FIGS. 2A, 2D, and 2G. Expression levels of various genes are shown in FIGS. 2B, 2C, 2E, 2F, 2H, and 2I. Total RNA samples were isolated on Day 6 without or with forskolin stimulation for 4 h. Expression of various genes was analyzed by quantitative real-time PCR (qPCR). Data were normalized to Control (Ctrl) and represent mean±SEM, **p<0.01, **p<0.01, ***p<0.001.

FIGS. 3A-3N illustrate that bexarotene-mediated adipogenesis in C2C12 cells depends on RXRα and RXRγ. FIG. 3A illustrates the adipogenic effect of bexarotene (10 μM), the RXR antagonist HX531 (10 μM), and the combination of Bex+HX531 upon C2C12 cells. Bexarotene and/or HX531 were added for the first 2 days during adipogenesis. ORO staining was performed on Day 6. FIG. 3B illustrates the effects of RXR agonist bexarotene compared to the RXR antagonist HX531 upon white adipose tissue (WAT) cells (3T3-L1 and C3H10T1/2) and upon pre-brown adipose tissue (BAT) cell differentiation, as detected by ORO staining. As shown, the RXR agonist bexarotene has an opposite effect upon browning compared to the RXR antagonist HX531. FIG. 3C illustrates that the adipogenic effect of bexarotene upon C2C12 cells was reduced by shRNA targeted inhibition of RXRα, RXRβ, or RXRγ. The shNT is a non-target shRNA used as a control. FIG. 3D graphically illustrates the knock-down efficiency of various RXR shRNA in C2C12 cells as detected by real-time PCR. The shRNA reduced the detected RNA levels of RXRα, RXRβ, and RXRγ. FIG. 3E illustrates that RXR overexpression potentiated the adipogenic effect of bexarotene in C2C12 cells as shown by ORO staining. FIG. 3F graphically illustrates the relative overexpression (OE) of RXRα, RXRβ, or RXRγ in different C2C12 cell lines, as detected by real-time PCR. Expression levels of cells transfected with the pMX vector (no RXR transgene) are shown for comparison with those that express RXRα, RXRβ, or RXRγ. FIG. 3G graphically illustrates mRNA levels of general adipocyte markers PPARγ and Adiponectin in C2C12 cells. Data were normalized to Control (Ctrl) and represent mean±SEM, *p<0.05, **p<0.01, ***p<0.001. FIG. 3H-1 graphically illustrates mRNA levels of brown-selective genes Ucp1, Prdm16, and PPARα in C2C12 cells and pre-BAT during reprogramming. FIG. 3H-2 graphically illustrates mRNA levels of brown-selective genes PPARγ, Cox7a1, and Cox8b in C2C12 cells and pre-BAT during reprogramming. Data were normalized to Control (Ctrl) and represent mean±SEM, *p<0.05, **p<0.01, ***p<0.001. FIG. 3H-3 graphically illustrates Ucp1 expression levels with or without forskolin stimulation for 4 h during bexarotene/RXR-mediated C2C12 adipogenic differentiation. Data were normalized to Ctrl and represent mean±SEM, *p<0.05, **p<0.01. FIG. 3I graphically illustrates bexarotene/RXR inhibition of myogenesis gene (Myf5, MyoD, MyoG) expression as detected by real-time PCR. Data were normalized to Control (Ctrl) and represent mean±SEM, *p<0.05, **p<0.01, ***p<0.001. FIG. 3J graphically illustrates Ucp1 expression levels in bexarotene/RXR-induced adipocytes obtained from mouse primary myoblasts. Data were normalized to Control (Ctrl) and represent mean±SEM, ***p<0.001. FIG. 3K shows bright-field images of bexarotene/RXR-induced adipogenesis in primary myoblasts purified from mouse limb muscles. FIG. 3L shows a bright-field image illustrating bexarotene/RXR-induced adipogenesis compared to control in C3H10T1/2 cells. Images of cells transfected with the pMX vector (no RXR transgene) are shown for comparison with those that express RXRα, RXRβ, or RXRγ. FIG. 3M graphically illustrates expression of white adipose tissue-specific genes Retn, Retn1a, and Psat1 in bexarotene/RXR-induced adipocytes from C3H10T1/2 cells. Data were normalized to Control (Ctrl) and represent mean±SEM, *p<0.05, **p<0.01, ***p<0.001. FIG. 3N-1 graphically illustrates expression of BAT-specific genes Ucp1, Prdm16, PRARα, and Pgc1α in bexarotene/RXR-induced adipocytes obtained from C3H10T1/2 cells. FIG. 3N-2 graphically illustrates expression of BAT-specific genes Cox7a1, Cox8b, and PPARγ n bexarotene/RXR-induced adipocytes obtained from C3H10T1/2 cells. Data were normalized to Control (Ctrl) and represent mean±SEM, *p<0.05, **p<0.01, ***p<0.001.

FIG. 4A-4J illustrate that bexarotene increases brown adipose tissue formation and function in vivo. FIG. 4A graphically illustrates food intake in saline-treated and bexarotene-treated mice (n=7). FIG. 4B graphically illustrates body weight gain in saline-treated and bexarotene-treated mice (n=7). *p<0.05. FIGS. 4C-1 and 4C-2 graphically illustrate oxygen consumption (VO₂) in saline-treated and bexarotene-treated mice (n=7). **p<0.01. FIGS. 4D-1 and 4D-2 graphically illustrates carbon dioxide release (VCO₂) in saline-treated and bexarotene-treated mice (n=7). **p<0.01. FIGS. 4E-1 and 4E-2 graphically illustrates heat release in saline-treated and bexarotene-treated mice (n=7). **p<0.01. FIGS. 4F-1, 4F-2 and 4F-3 illustrate brown adipose tissue (BAT) mass in saline-treated and bexarotene-treated mice (n=7). *p<0.05. FIGS. 4G-1 and 4G-2 graphically illustrate white adipose tissue (WAT) mass in saline-treated and bexarotene-treated mice (n=7). FIG. 4H shows images of brown adipose tissue (BAT), subcutaneous inguinal white adipose tissue (subWAT), and epididymal white adipose tissue (epiWAT) in bexarotene-treated mice and Control (Ctrl) after haematoxylin and eosin staining of representative sections of BAT, subWAT and epiWAT tissues. FIG. 4I graphically illustrates expression of brown adipose tissue-related genes in brown adipose tissue (BAT), in subcutaneous/inguinal white adipose tissue (subWAT), and in epididymal white adipose tissue (epiWAT). *p<0.05, **p<0.01. FIGS. 4J1 and 4J2 illustrate RXRα, RXRβ, and RXRγ expression in white adipose tissue (WAT), brown adipose tissue (BAT), and Skeletal Muscle (SKM). mRNA were purified in WATs, BAT and SKM from mice (n=3). Data were normalized to RXRα levels in epididymal white adipose tissues (epiWAT) and represent mean±SEM, *p<0.05, ***p<0.001.

FIG. 5A-5E show that bexarotene/RXR-mediated brown adipogenesis partially depends upon PRDM16. FIG. 5A-1 illustrates cell morphology changes in bexarotene/RXRα-treated C2C12 cells during adipogenesis. FIG. 5A-2 graphically illustrates Ucp1 and Prdm16 expression levels in bexarotene/RXRα-treated C2C12 cells during adipogenesis. FIG. 5B graphically illustrates Ucp1 and Prdm16 levels in bexarotene/RXR-treated C2C12 cells on Day 2. Data were normalized to Control (Ctrl) and represent mean±SEM, *p<0.05, **p<0.01, ***p<0.001. FIG. 5C-1 to 5C-3 shows that a shRNA directed against PRDM16 partially inhibited bexarotene/RXR-induced adipogenesis in C2C12 cells. Ucp1 and PPARγ expression levels were evaluated on Day 6. FIGS. 5D-1 and 5D-2 show that bexarotene and PRDM16 synergistically induce UCP1 expression in primary myoblasts. Data were normalized to control (Ctrl) and represent mean±SEM, ***p<0.001. FIG. 5E-1 to FIG. 5E-6 graphically illustrate mRNA expression levels in C3H10T1/2 cells treated with Bex and/or various types of RXR. FIG. 5E-1 shows UCP1 expression. FIG. 5E-2 shows PGC1α expression. FIG. 5E-3 illustrates Cox7a1 expression. FIG. 5E-4 shows PPARα expression. FIG. 5E-5 shows AP2 expression. FIG. 5E-6 shows Prdm16 expression.

FIG. 6A-6E illustrate that bexarotene initiates ‘browning’ pathways at an early stage of adipogenesis in C2C12 cells. FIG. 6A is a scatter map of gene expression in control (Ctrl) and bexarotene-treated adipogenic samples. Genes up-regulated by bexarotene (lighter dots) and those down-regulated (darker dots) are shown. FIG. 6B illustrates the enriched Wikipathways of differentially expressed genes in GO-Elite as visualized in Cytoscape. FIG. 6C graphically illustrates mRNA levels of some adipogenesis and browning genes in C2C12 cells on Day 2 of bexarotene treatment as detected by real-time PCR. Data were normalized to Control (Ctrl) and represent mean±SEM, *p<0.05, **p<0.01, ***p<0.001. FIG. 6D graphically illustrates mRNA levels of some adipogenesis and browning genes in C2C12 cells on Day 2 of bexarotene treatment as detected by real-time PCR. Data were normalized to Control (Ctrl) and represent mean±SEM, *p<0.05, **p<0.01, ***p<0.001. FIG. 6E illustrates early stage bexarotene RXR-mediated browning adipogenesis pathways in myoblasts.

FIG. 7A-7B illustrate the effects of compounds Ciclopirox (CPX) and IOX2 upon expression of brown adipose tissue markers Ucp1, Pgc1α, Tbx15, and PPARγ. FIG. 7A-1 graphically illustrates the effects of Ciclopirox upon Ucp1, Pgc1α, and Tbx15 expression levels in the C3H10T1/2 cells. FIG. 7A-2 shows images of Ciclopirox-treated and untreated C3H10T1/2 cells. FIG. 7A-3 shows the structure of Ciclopirox. FIG. 7B-1 graphically illustrates the effects of IOX2 upon Ucp1, Pgc1α, Pgc1γ, and Tbx15 expression levels in the C3H10T1/2 cells. FIG. 7B-2 shows images of IOX2-treated and untreated C3H10T1/2 cells. FIG. 7A-3 shows the structure of IOX2.

FIG. 8A-8E graphically illustrate weight and blood glucose levels in mice fed a high fat diet (HFD) with and without bexarotene treatment. FIG. 8A graphically illustrates body weight of mice administered bexarotene compared to control mice who did not. FIG. 8B graphically illustrates that bexarotene-treated mice had less subcutaneous fat than control mice that did not receive bexarotene. FIG. 8C graphically illustrates that bexarotene-treated mice had less epididymal adipose tissue than control mice that did not receive bexarotene. FIG. 8D shows that bexarotene-treated mice responded to insulin more sensitively as detected by insulin tolerance tests (ITT). FIG. 8E shows that bexarotene-treated mice have improved glucose homeostasis upon glucose challenge as assessed by glucose tolerance testing (GTT).

### DETAILED DESCRIPTION

Methods and compositions are described herein for reprogramming cells to cross lineage boundaries and to directly convert into brown adipose cells. The methods and compositions include use of bexarotene, ciclopirox, IOX2, or combinations thereof, which have the following structures.

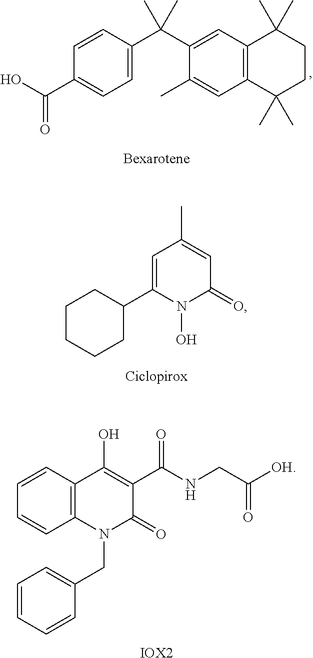

As described herein, the inventors have found that bexarotene, ciclopirox, IOX2, or combinations thereof, can modulate the phenotype of the cell. Specifically, addition or administration of bexarotene, ciclopirox, IOX2, or combinations thereof, to various cell types can convert the cells into brown adipocytes. The brown adipocytes so generated can engage in thermogenesis, and exhibit increased oxygen consumption and increased carbon dioxide release. When administered to mammals, the mammals exhibit reduced weight gain, even though food intake is the same as mammals who did not receive bexarotene. Mammals receiving bexarotene, ciclopirox, IOX2, or combinations thereof, had increased brown adipose mass and exhibited increased activity. White adipose tissue-related gene expression is also inhibited in cells contacted with bexarotene, ciclopirox, IOX2, or combinations thereof.

### Cellular Conversion to Brown Adipocytes/Brown Adipose Tissue

Methods of inducing a brown adipose tissue (BAT) generation from selected (non-BAT) cells are described herein. The method involves contacting selected cells with bexarotene, ciclopirox, IOX2, or combinations thereof. The method can, for example, be performed in vitro, ex vivo, or in vivo.

When performing the method in vitro or ex vivo the selected cell(s) can be contacted with a variety of bexarotene, ciclopirox, or IOX2 concentrations. For example, the cells can be incubated in a culture containing about 0.01 μM to about 100 μM bexarotene, ciclopirox, IOX2, or combinations thereof, or about 0.1 μM to about 90 μM bexarotene, ciclopirox, IOX2, or combinations thereof, or about 0.05 μM bexarotene to about 50 μM bexarotene, ciclopirox, IOX2, or combinations thereof. Experiments described herein illustrate, for example, that 1-10 μM bexarotene, ciclopirox, IOX2, or combinations thereof, induce selected cells to become brown adipose tissues. Many different cell types can be converted to brown adipocytes and/or brown adipose tissues. For example, the cells selected for such conversion can be myoblasts, adipocytes, pre-adipocytes, white adipocytes, mesenchymal precursor cells, multipotent stem cells, pluripotent stem cells, unipotent stem cells, fibroblasts, and combinations thereof. Other types of cells that can be used in the methods of the invention and converted into brown adipose cells are described hereinbelow.

The selected cells can be obtained from a subject to whom the cells (after treatment as described herein) are later returned. Hence, the cells can be autologous cells. Alternatively, the cells can be allogeneic cells (relative to a subject to be treated or who may receive the cells).

The methods can also be performed in vivo on a subject, such as a mammal. Such methods can involve administering bexarotene, ciclopirox, IOX2, or combinations thereof, to the subject. In some instances an effective amount of bexarotene, ciclopirox, IOX2, or combinations thereof, is administered to the subject. Such an effective amount induces conversion of cells to brown adipocytes or brown adipose tissue within the subject. The effective amount of bexarotene, ciclopirox, IOX2, or combinations thereof, increases expression of brown adipose-related tissues. The effective amount of bexarotene, ciclopirox, IOX2, or combinations thereof, decreases expression of white adipose-related tissues. Examples of bexarotene, ciclopirox, IOX2, or combinations thereof, dosages include about 1 mg/kg/day to about 1000 mg/kg/day, or about 5 mg/kg/day to about 500 mg/kg/day, or about 10 mg/kg/day to about 200 mg/kg/day, or about 25 mg/kg/day to about 100 mg/kg/day. As illustrated herein, subjects receiving 50 mg/kg/day.

The bexarotene, ciclopirox, and/or IOX2 compositions can be administered or incubated with selected cells for a time sufficient to induce expression of brown adipose tissue-related genes. The compositions can be administered or incubated with selected cells for a time sufficient to reduce white adipose tissue-related genes. For example, compositions can be administered or incubated with selected cells for a time sufficient to induce expression of Ucp1, Pgc1α, PPARγ, PPARδ, Prdm16, adiponectin, Cox7a1, Cox8b, Myf5, MyoD, MyoG, Tbx15, and any combinations thereof.

Bexarotene, ciclopirox, IOX2, or combinations thereof, can also be administered or incubated with selected cells for a time sufficient to reduce expression of white adipose tissue-related genes. Such white adipose tissue-related genes include resistin (Retn), resistin-like alpha (retn1α), phosphoserine aminotransferase 1 (Psat1), and any combination thereof.

The bexarotene, ciclopirox, and/or IOX2 compositions can be administered to subjects for a time sufficient to induce thermogenesis, increase oxygen consumption, increase carbon dioxide release, reduce weight gain (e.g, without significant reduction in food intake), increase brown adipose mass, increase physical activity, reduce white adipose tissue mass. The compositions and methods can be employed by a subject indefinitely, or from about 1 day to about 48 months, or from about 1 week to about 36 months, or from about 2 weeks to about 24 months, or from about 3 weeks to about 18 months, or from about 1 month to about 12 months.

For example, the selected cells (e.g., starting cells) can be treated for a time sufficient to convert those cells into brown adipocytes or brown adipose tissue. Such treatment can include incubation in media containing bexarotene. Such a time can be about 4 hours to about 14 days, or about 6 hours to about 10 days, or for about 8 hours to about 7 days, or for about 12 hours to about 5 days, or about 12 hours to about 3 days, or for about 1 to 3 days.

The starting cells can be incubated with bexarotene, ciclopirox, IOX2, or combinations thereof, in a cell culture medium. The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are available to those skilled in the art.

Examples of commercially available media include, but are not limited to, Dulbecco's Modified Eagle's Medium (DMEM), Minimal Essential Medium (MEM), Basal Medium Eagle (BME), RPM1 1640, Ham's F-10, Ham's F-12, Minimal Essential Medium alpha (αMEM), Glasgow's Minimal Essential Medium (G-MEM), Iscove's Modified Dulbecco's Medium, or a general purpose media modified for use with pluripotent cells, such as X-VIVO (Lonza).

The starting cells can be dispersed in a cell culture medium that contains bexarotene, ciclopirox, IOX2, or combinations thereof, at a cell density that permits cell expansion. For example, about 1 to 10¹⁰ cells can be contacted with bexarotene, ciclopirox, IOX2, or combinations thereof, in a selected cell culture medium, especially when the cells are maintained at a cell density of about 1 to about 10⁸ cells per milliliter, or at a density of about 100 to about 10⁷ cells per milliliter, or at a density of about 1000 to about 10⁶ cells per milliliter.

Such methods can be used to generate a population of brown adipocytes or brown adipose tissue that can be transplanted into a subject or used for experimentation.

In some embodiments, population of brown adipocytes or brown adipose tissue can be frozen at liquid nitrogen temperatures, stored for periods of time, and then thawed for use at a later date. If frozen, a population of brown adipocytes or brown adipose tissue can be stored in a 10% DMSO, 50% FCS, 40% RPMI 1640 medium. Once thawed, the cells can be expanded by culturing the cells in an appropriate medium that can contain selected growth factors, vitamins, feeder cells, and other components selected by a person of skill in the art.

### Selected Cells for Conversion to Brown Adipose Tissue

A selected cell type or population of cells for conversion to brown adipose tissue can be derived from essentially any source, and can be heterogeneous or homogeneous. In certain embodiments, the cells to be treated as described herein are adult cells, including essentially any accessible adult cell type(s). In other embodiments, the cells used are adult stem cells, progenitor cells, or somatic cells. In still other embodiments, the cells treated with any of the compositions and/or methods described herein include any type of cell from a newborn, including, but not limited to newborn cord blood, newborn stem cells, progenitor cells, and tissue-derived cells (e.g., somatic cells). Accordingly, a starting or selected population of cells that is reprogrammed by the compositions and/or methods described herein, can be essentially any live somatic cell type. As illustrated herein, myoblasts, mesenchymal cells, and fibroblasts can be reprogrammed to cross lineage boundaries and to be converted into brown adipose cells.

Various cell types from all three germ layers have been shown to be suitable for somatic cell reprogramming include, but not limited to myoblasts, adipocytes, pre-adipocytes, mesenchymal precursor cells, multipotent stem cells, pluripotent stem cells, unipotent stem cells, fibroblasts, and any combination thereof. Other types of cells that can be converted to brown adipocytes include liver and stomach, pancreatic β cells, human dermal fibroblasts, meningiocytes, stem cells. Such cells can be used in the compositions and methods described herein.

The cells can be autologous or allogeneic cells (relative to a subject to be treated or who may receive the cells).

### Treatment

The compositions containing bexarotene, ciclopirox, IOX2, or combinations thereof, with or without converted brown adipocytes and/or brown adipose tissue that are described herein, can be employed in a method of treating a subject. The compositions and methods can be employed for inducing weight loss in a subject. Such methods can include administering an effective amount bexarotene, ciclopirox, IOX2, or combinations thereof, to the subject; wherein the effective amount of bexarotene, ciclopirox, IOX2, or combinations thereof, is an amount sufficient to induce a brown adipose tissue-like phenotype in cells of the subject. For example, such a method can reduce the mass of white adipose tissue in the subject and/or increase the mass of brown adipose tissue in the subject.

As described herein, administration of bexarotene, ciclopirox, IOX2, or combinations thereof, causes various cell types (including white adipose tissue) to assume a brown adipose tissue-like phenotype. One effect of such a method is to reduce the body weight of subjects who have undergone treatment. Another effect of such a method is an increase of thermogenesis in the subject. As demonstrated herein, the increase of thermogenesis in a subject can increase oxygen consumption and carbon dioxide respiration. Such thermogenesis can also increase the ability of the subject to maintain core body temperature. Accordingly, in some embodiments, a subject in need of treatment according to the methods described herein can be a subject selected from the group of: a subject in need of an increased body temperature; a subject in need of treatment or prevention of exposure to low temperatures; and a subject in need of treatment or prevention of hypothermia.

In some embodiments of any of the foregoing aspects, a therapeutically effective amount of bexarotene, ciclopirox, IOX2, or combinations thereof, can be an amount that does not substantially reduce lean body mass of the subject.

In some embodiments, the methods described herein are used to treat a subject having or diagnosed as excess body fat and/or obesity with bexarotene, ciclopirox, IOX2, or combinations thereof. Subjects having obesity can be identified by a physician using current methods of diagnosis, for example, by determining the body mass index (BMI) of a subject. This can include, but is not limited to, a subject diagnosed as having and/or at risk of having or developing type II diabetes, metabolic syndrome, insulin resistance, cardiac disease, early-onset myocardial infarction, osteoarthritis, gout, heart disease, gall bladder disease, fatty liver disease, sleep apnea, gall stones, and numerous types of cancer. Also envisioned is the treatment of patients who desire treatment for aesthetic reasons (i.e. to maintain a desired weight, BMI, or appearance) even if they are at a healthy weight or BMI prior to treatment. Risk factors which can increase the likelihood of a subject being at risk of having or developing a higher than desired BMI include a high caloric intake, sedentary lifestyle, hypothyroidism and a family history of high BMI or obesity.

In some embodiments, the methods and compositions described herein can be used to treat a human subject. In some embodiments, the methods and compositions described herein can relate to the treatment of a domesticated animal, an experimental animal, a zoo animal, or a companion animal. Examples of animals that can be treated or tested using the compositions and/or methods described herein include dogs, cats, pigs, goats, cattle, horses, rats, mice, rabbits, poultry, pigeons, monkeys, and apes.

The compositions and methods described herein can be administered to a subject having or diagnosed as having obesity. In some embodiments, the methods described herein include administering an effective amount of a composition containing bexarotene, ciclopirox, IOX2, or combinations thereof, to a subject in order to alleviate a symptom of obesity.

As used herein, “alleviating a symptom of a condition is ameliorating any symptom associated with the condition. As compared with an equivalent untreated control, such reduction is by at least 5%, 10%, 20%, 40%, 50%, 60%, 80%, 90%, 95%, 99% or more as measured by any standard technique.

A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, or injection. In some embodiments, the administration can be intraperitoneal, oral, and/or intravenous. Administration can be local or systemic, as described in further detail below.

### Administration of Brown Adipocytes and Brown Adipose Tissue Cells

Cells generated as described herein can be employed to increase the mass of brown adipose tissue in the subject, to treat emaciation, and to treat or prevent reactions to low temperatures. The cells are administered in a manner that permits them to graft or migrate to various tissue sites and to reconstitute or regenerate various areas. The cells may be administered to a recipient by local injection, or by systemic injection. In some embodiments, the cells can be administered parenterally by injection into a convenient cavity.

Injected cells can be traced by a variety of methods, for example, for experimental purposes. Cells containing or expressing a detectable label (such as green fluorescent protein, or beta-galactosidase) can readily be detected. The cells can be pre-labeled, for example, with BrdU or [³H] thymidine, or by introduction of an expression cassette that can express green fluorescent protein, or beta-galactosidase. Alternatively, the reprogrammed cells can be detected by their expression of a cell marker that is not expressed by the animal employed for testing (for example, a human-specific antigen). The presence and phenotype of the administered population of reprogrammed cells can be assessed by fluorescence microscopy (e.g., for green fluorescent protein, or beta-galactosidase), by immunohistochemistry (e.g., using an antibody against a human antigen), by ELISA (using an antibody against a human antigen), or by RT-PCR analysis using primers and hybridization conditions that cause amplification to be specific for human polynucleotides.

A population of cells can be introduced by injection, catheter, implantable device, or the like. A population of cells can be administered in any physiologically acceptable excipient or carrier that does not adversely affect the cells.

A population of cells can be supplied in the form of a pharmaceutical composition. Such a composition can include an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000. The choice of the cellular excipient and any accompanying constituents of the composition that includes a population of reprogrammed cells can be adapted to optimize administration by the route and/or device employed.

A composition that includes a cell population can also include or be accompanied by one or more other ingredients that facilitate engraftment or functional mobilization of the cells. Suitable ingredients include matrix proteins that support or promote adhesion of the cells, or complementary cell types, such as cardiomyocytes or muscle cells. In another embodiment, the composition may include physiologically acceptable matrix scaffolds. Such physiologically acceptable matrix scaffolds can be resorbable and/or biodegradable.

The cell population generated by the methods described herein can include low percentages of non-brown adipose cells (e.g., low percentages or no fibroblasts or white adipose cells). For example, a population of cells for use in compositions and for administration to subjects can have less than about 90% non-brown adipose cells, less than about 85% non-brown adipose cells, less than about 80% non-brown adipose cells, less than about 75% non-brown adipose cells, less than about 70% non-brown adipose cells, less than about 65% non-brown adipose cells, less than about 60% non-brown adipose cells, less than about 55% non-brown adipose cells, less than about 50% non-brown adipose cells, less than about 45% non-brown adipose cells, less than about 40% non-brown adipose cells, less than about 35% non-brown adipose cells, less than about 30% non-brown adipose cells, less than about 25% non-brown adipose cells, less than about 20% non-brown adipose cells, less than about 15% non-brown adipose cells, less than about 12% non-brown adipose cells, less than about 10% non-brown adipose cells, less than about 8% non-brown adipose cells, less than about 6% non-brown adipose cells, less than about 5% non-brown adipose cells, less than about 4% non-brown adipose cells, less than about 3% non-brown adipose cells, less than about 2% non-brown adipose cells, or less than about 1% non-brown adipose cells of the total cells in the cell population.

In some instances the population of cells for use in compositions and for administration to subjects can have at least about 60% brown adipose cells, at least about 70% brown adipose cells, at least about 75% brown adipose cells, at least about 80% brown adipose cells, at least about 85% brown adipose cells, at least about 90% brown adipose cells, at least about 95% brown adipose cells, at least about 97% brown adipose cells, or at least about 98% brown adipose cells.

The cell population generated by the methods described herein can include other cell types that facilitate the function of brown adipose cells. For example, the cell population can include myoblasts, skeletal muscle cells, and/or cardiomyocytes.

The brown adipose cells can be inserted into such a delivery device, e.g., a syringe or an implant. Such an implant can release the cells in a bolus or over time.

Reprogrammed (brown adipose) cells can be included in the compositions in varying amounts depending upon the disease or injury to be treated. The reprogrammed cells can be administered in a “therapeutically effective amount.” Such a therapeutically effective amount is an amount sufficient to obtain the desired physiological effect, e.g., increased thermogenesis, increased oxygen consumption, reduced weight gain, increased brown adipose mass, increased physical activity, reduced white adipose tissue, loss of weight, or a combination thereof.

For example, the compositions can be prepared in liquid form for local or systemic administration containing about 10³ to about 10¹² reprogrammed (brown adipose) cells, or about 10⁴ to about 10¹⁰ reprogrammed (brown adipose) cells, or about 10⁵ to about 10⁸ reprogrammed (brown adipose) cells. The compositions can contain at least about 10⁴ reprogrammed (brown adipose) cells, or at least about 10⁵ reprogrammed (brown adipose) cells, at least about 10⁶ reprogrammed (brown adipose) cells, at least about 10⁷ reprogrammed (brown adipose) cells, at least about 10⁶ reprogrammed (brown adipose) cells, at least about 10⁷ reprogrammed (brown adipose) cells, or at least about 10⁸ reprogrammed (brown adipose) cells.

Administration of the cells can be via a single dose, in multiple doses, in a continuous or intermittent manner, depending, for example, upon the recipient's physiological condition, whether the purpose of the administration is therapeutic or prophylactic, and other factors known to skilled practitioners. The administration of the cells thereof can be in a series of spaced doses. Both local and systemic administration is contemplated.

### Compositions

The invention also relates to compositions containing bexarotene, ciclopirox, IOX2, or any combination thereof. The compositions can a carrier or excipient.

The compositions described herein can be pharmaceutical compositions. In other embodiments, the compositions are used as diagnostic imaging compositions. In some embodiments, the compositions can include a pharmaceutically acceptable carrier. By “pharmaceutically acceptable” it is meant a carrier, diluent, excipient, and/or salt that is compatible with the other ingredients of the formulation, and not deleterious to the recipient thereof.

In some embodiments, the bexarotene, ciclopirox, IOX2, or combinations thereof, and other ingredients can be administered in a “therapeutically effective amount.” Such a therapeutically effective amount is an amount sufficient to obtain the desired physiological effect, e.g., increased thermogenesis, increased oxygen consumption, reduced weight gain, increased brown adipose mass, increased physical activity, reduced white adipose tissue, loss of weight, conversion of a cell or population of cell into a brown adipose cell or a population of cells, or a combination thereof.

To achieve the desired effect(s), the bexarotene, ciclopirox, IOX2, or combinations thereof, and other ingredients can be administered as single or divided dosages.

For example, bexarotene, ciclopirox, IOX2, or combinations thereof, can be administered in dosages of at least about 0.01 mg/kg to about 750 mg/kg, of at least about 0.01 mg/kg to about 500 mg/kg, at least about 0.1 mg/kg to about 300 mg/kg or at least about 1 mg/kg to about 100 mg/kg of body weight, although other dosages may provide beneficial results. The amount administered will vary depending on various factors including, but not limited to, the severity of disease, the weight, the physical condition, the health, and the age of the mammal. Such factors can be readily determined by the clinician employing animal models or other test systems that are available in the art.

The bexarotene, ciclopirox, IOX2, or combinations thereof, compositions can be used to treat a human patient or other subjects in need of such treatment. The compositions are administered in a manner that permits the bexarotene, ciclopirox, IOX2, or combinations thereof, to become localized or to migrate to a diseased site. Devices are available that can be adapted for administering bexarotene, for example, to sites with cells selected for conversion to brown adipose cells. Bexarotene, ciclopirox, IOX2, or combinations thereof, can be administered locally or systemically. Compositions of bexarotene, ciclopirox, IOX2, or combinations thereof, can be introduced by injection, catheter, implantable device, or the like. For example, the compositions can be administered in any physiologically acceptable excipient or carrier that does not adversely affect the bexarotene ciclopirox, IOX2, and other ingredients. For example, compositions can be administered orally, intravenously, parenterally, into white adipose tissue, into muscle, intra-abdominally, and the like. Methods of administering bexarotene, ciclopirox, IOX2, or combinations thereof, and compositions thereof to subjects, particularly human subjects, include ingestion or injection into any such target sites in the subjects.

Bexarotene, ciclopirox, IOX2, or combinations thereof, can be included in the compositions in varying amounts depending upon the weight and condition of the subject, as well as the extent of cellular reprogramming to be achieved.

Administration of the bexarotene, ciclopirox, IOX2, or combinations thereof, can be via a single dose, in multiple doses, in a continuous or intermittent manner, depending, for example, upon the recipient's physiological condition, whether the purpose of the administration is therapeutic or prophylactic, and other factors known to skilled practitioners. The administration of the compositions thereof can be essentially continuous over a preselected period of time or can be in a series of spaced doses. Both local and systemic administration is contemplated.

To prepare the composition bexarotene, ciclopirox, IOX2, or/and other agents are synthesized or otherwise obtained, and purified as necessary or desired. Non-labile components can be lyophilized. However, the bexarotene, ciclopirox, IOX2, and/or other ingredients can be maintained in a solution, medium, liquid carrier, solid matrix, or semi-solid carrier that does not adversely affect their viability. The components can be stabilized, for example, by addition of chelating agents, physiological salts, and the like. These agents can be adjusted to the appropriate concentration, and optionally combined with other agents.

The absolute weight of bexarotene, ciclopirox, and/or IOX2, to be administered in a unit dose can vary widely. For example, about 0.01 to about 10 g, or about 0.1 to about 5 g, of bexarotene, ciclopirox, and/or IOX2 can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 10 g, from about 0.5 g to about 8 g, or from about 0.5 g to about 5 g.

Daily, bi-weekly, and weekly doses of bexarotene, ciclopirox, and/or IOX2can vary as well. Such daily doses can range, for example, from about 0.1 g/day to about 50 g/day, from about 0.1 g/day to about 25 g/day, from about 0.1 g/day to about 15 g/day, from about 0.5 g/day to about 10 g/day, from about 0.5 g/day to about 8 g/day, and from about 0.5 g/day to about 5 g/day.

Thus, one or more suitable unit dosage forms comprising bexarotene, ciclopirox, and/or IOX2 can be administered by a variety of routes including parenteral (including subcutaneous, intravenous, intramuscular and intraperitoneal), oral, rectal, dermal, transdermal, intrathoracic, intrapulmonary and intranasal (respiratory) routes. The bexarotene, ciclopirox, and/or IOX2 can also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods can include the step of mixing the therapeutic agent with liquid carriers, solid matrices, semi-solid carriers, finely divided solid carriers or combinations thereof, and then, if necessary, introducing or shaping the product into the desired delivery system.

The compositions can be prepared in many forms that include aqueous solutions, suspensions, tablets, hard or soft gelatin capsules, and liposomes and other slow-release formulations, such as shaped polymeric gels.

The bexarotene, ciclopirox, and/or IOX2 can be administered in an oral dosage form for release into the stomach or for release into the intestine after passing through the stomach. Such formulations are described in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives.

The compositions can also include retinoids and/or retinoic acid receptor agonists other than bexarotene. Examples include retinoic acid, 9-cis retinoic acid, all-trans 3,4-didehydro retinoic acid, 4-oxo retinoic acid and retinol. Other examples are described in PCT application publication WO/2013/052647 by Makra, published Apr. 11, 2013, which is incorporated herein by reference in its entirety. For example, the compositions can include retinoids such as those embraced by formula I.

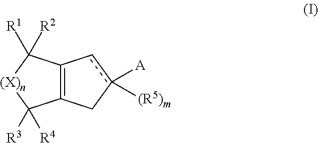

wherein the dotted bond is either present and forms a double bond, or is absent; R¹, R², R³ and R⁴ are independently hydrogen or alkyl; n is 1, 2 or 3; X is —C(R⁸)(R⁹)— for n=1, 2 or 3; or X is oxygen for n=1; R⁸ and R⁹ are independently hydrogen or alkyl; R is hydrogen, alkyl, alkoxy, alkoxy-alkyl-, alkylthio, alkyl-NR¹⁰—, alkenyl, alkenyloxy, alkynyl, benzyl, cycloalkyl-alkyl, phenyl-alkyl, R¹⁰ is hydrogen or alkyl; m is 0 when the dotted bond is present; and m is 1 when the dotted bond is absent; and A is a residue of formula II:

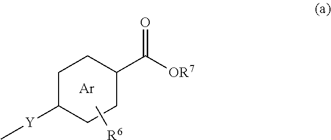

or of formula III:

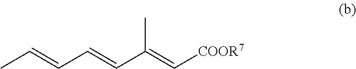

wherein Ar is phenyl or a heteroarylic ring; R⁶ is hydrogen, halogen, alkoxy or hydroxy; R⁷ is hydrogen or alkyl; and Y is —COO—, —OCO—, —CONR¹⁰—, —NR¹⁰CO—, —CH═CH—, —C≡C—, —COCH═CH—, —CHOHCH═CH—, —CH₂O—, —CH₂S—, —CH₂SO—, —CH₂S0₂-, —CH₂NR¹⁰—, —OCH₂—, —SCH₂—, —SOCH₂—, —SO₂CH₂— or —NR¹⁰CH₂—, with the proviso that when Y is —OCO—, —NR¹⁰CO—, —OCH₂—, —SCH₂—, —SOCH₂—, —SO₂CH₂— or —NR¹⁰CH₂—, R⁵ is hydrogen, alkyl, alkoxy-alkyl-, alkenyl, alkynyl, benzyl, cycloalkyl-alkyl or phenyl-alkyl; and pharmaceutically active salts of carboxylic acids of formula I.

The compounds described herein and other selected ingredients can be formulated for parenteral administration (e.g., by injection, for example, bolus injection or continuous infusion) and may be presented in unit dosage form in ampoules, prefilled syringes, small volume infusion containers or multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Suitable carriers include saline solution and other materials commonly used in the art.

The compositions can also contain other ingredients such as vitamins, anti-microbial agents, or preservatives.

### Kits

A variety of kits are described herein that include any of the compositions, compounds and/or agents described herein. The compounds and/or agents described herein can be packaged separately into discrete vials, bottles or other containers. Alternatively, any of the compounds and/or agents described herein can be packaged together as a single composition, or as two or more compositions that can be used together or separately. The compounds and/or agents described herein can be packaged in appropriate ratios and/or amounts to facilitate conversion of selected cells to brown adipose tissue.

A kit is described herein for culture of cells in vitro or ex vivo that can include any of the compositions, compounds and/or agents described herein, as well as instructions for using those compositions, compounds and/or agents. Some kits can include a cell culture or cell media that includes any of the compositions, compounds and/or agents described herein. The kits can include one or more sterile cell collection devices such as a swab, skin scrapping device, a needle, a syringe, and/or a scalpel. The kits can also include antibodies for detection of brown adipose cell markers or white adipose cell markers such as antibodies against Ucp1, PPARα, Prdm16, Pgc1α, PPARγ, Cox7a1, Cox8b, Retn, Retnia, Psat1, or any combination thereof. The antibodies can be labeled so that a detectable signal can be observed when the antibodies form a complex with the cell marker(s).

The instructions can include guidance for culturing cells for a time and under conditions sufficient to convert a selected cell across differentiation boundaries and into the brown adipose lineage. For example, the instructions can describe amounts of the compositions, compounds and/or agents described herein to add to cell culture media, times sufficient to convert cells to the brown adipose lineage, maintenance of appropriate cell densities for optimal conversion, and the like. For example, the instructions can describe procedures for rehydration or dilution of the compositions, compounds and/or agents described herein. When a kit provides a cell culture medium containing some of the compositions, compounds and/or agents described herein, the instructions can describe how to add other compounds and/agents. The instructions can also describe how to convert the selected cells to brown adipocytes or to mature brown adipose tissue.

The instructions can also describe procedures for detecting brown adipose cell markers or white adipose cell markers by use of antibodies against those markers so that the extent of conversion and/or differentiation can be assessed.

Another kit is also described herein that includes any of the compositions, compounds and/or agents described herein for therapeutic treatment of a subject. The kit can include any of the compositions, compounds and/or agents described herein, as well as instructions for administering those compositions, compounds and/or agents. Such instructions can provide the information described throughout this application. The kit can also include cells. For example, the kit can include brown adipose tissues or cells that have been generated by the methods described herein and that are ready for administration.

The cells, compositions and/or compounds can be provided within any of the kits in a delivery device. Alternatively a delivery device can be separately included in the kit(s), and the instructions can describe how to assemble the delivery device prior to administration to a subject. The delivery device can provide sustained release of any of the compositions described herein. Alternatively or in addition, the delivery device can provide a scaffold for cell growth and/or a matrix for controlled release of any of the compositions, compounds or agents described herein.

Any of the kits can also include syringes, catheters, scalpels, sterile containers for sample or cell collection, diluents, pharmaceutically acceptable carriers, and the like. The kits can provide other factors such as any of the supplementary active ingredients.

### Definitions

The term “adipose tissue” refers to loose connective tissue which stores fat and is composed of multiple cell types, including adipocytes and microvascular cells. Adipose tissue also comprises stem and progenitor cells and endothelial precursor cells. Two varieties of adipose tissue are found in mammals; white adipose tissue and brown adipose tissue.

As the name would imply, white adipose tissue (WAT) comprises white adipocytes, which are adipocytes comprising a single large fat droplet, with a flattened nucleus located on the periphery of the cell.

White adipose tissue can help maintain body temperature (via insulation) and it stores energy in the form of lipids. In addition to morphology, white adipose tissue can be identified by the expression of white adipose tissue-related genes. White adipose tissue-related genes include, by way of non-limiting example, lipoprotein lipase (LPL; NCBI Gene ID No. 4023), hormone-sensitive lipase (HSL; NCBI Gene ID No. 3991), adiponectin (ADIPOQ NCBI Gene ID No. 9370), FABP4 (NCBI Gene ID No. 2167), CEBPA (NCBI Gene ID No. 1050), and PPARG2 (NCBI Gene ID No. 5468). White adipose tissue can be visceral white adipose tissue (also known as abdominal fat, organ fat, or intra-abdominal fat) or subcutaneous fat. Visceral fat is located in the abdominal cavity, typically between the organs (e.g. stomach, liver, kidneys, etc.) An excess amount of visceral white adipose tissue is correlated with central obesity and is linked to type 2 diabetes, insulin resistance, inflammatory disease, and additional obesity-related conditions. In some embodiments, white adipose tissue can be visceral white adipose tissue. Subcutaneous fat is found in the hypodermis just below the skin.

In contrast to white adipose tissue, brown adipose tissue (BAT) includes brown adipose cells that utilize the chemical energy in lipids and glucose to produce heat via non-shivering thermogenesis. Brown adipose cells can have multiple lipid droplets throughout the cell, a rounded nucleus and a large number of mitochondria, which give the cells their distinctive brown color. Brown adipose tissue-related genes include, by way of non-limiting example, lipoprotein lipase (LPL), UCP1 (NCBI Gene ID No. 7350), ELOVL3 (NCBI Gene ID No. 83401), PGC1A (NCBI Gene ID No. 10891), CYC1 (NCBI Gene ID No. 1537), CEBPA, PPARγ2, CYCS (NCBI Gene ID No. 54205), PRDM16 (NCBI Gene ID No. 63976), CIDEA (NCBI Gene ID No. 1149), COX4 (NCBI Gene ID No. 1327), TFAM (NCBI Gene ID No. 7019), and NRF1 (NCBI Gene ID No. 4899). Brown adipocytes can be distinguished from white adipocytes by having high relative expression of, by way of non-limiting example, UCP1, ELOVL3, PGC1A, and CYC1 and low relative expression of, by way of non-limiting example, ADIPOO, HSL, and FABP4, while both cell types will display high levels of PPARγ2 and LPL expression. Brown adipocytes are also characterized by RXR expression, RXR activity, UCP-1 expression, thermogenesis, and uncoupled mitochondrial respiration.

A number of markers, characteristics, and/or parameters of brown adipose tissue are described herein, particularly those that distinguish it from white adipose tissue. As used herein, a “brown adipose tissue-like” or “BAT-like” phenotype refers to a phenotype in which a cell (or tissue) displays a level of at least one marker, characteristic and/or parameter which differs between brown adipose tissue and white adipose tissue such that the level of the marker, characteristic and/or parameter deviates (in a statistically significant amount) from the level of that marker and/or parameter in a white adipose tissue reference level so that the cell (or tissue) more closely resembles brown adipose tissue than does the white adipose tissue reference level for at least one marker, characteristic, and/or parameter. For example, a white adipose tissue cell which is treated according to the methods described herein and which thereafter displays a statistically significant increase in thermogenesis as compared to a white adipose tissue reference level is a cell which has been modulated to display a brown adipose tissue-like phenotype. In some embodiments, the statistically significant amount is a change of at least 10% relative to the white adipose tissue reference level, e.g. 10% or more, 20% or more, 30% or more, 40% or more, 50% or more, 60% or more, 70% or more, 80% or more, 90% or more, 100% or more relative to the white adipose tissue control.

As used herein, “white adipose tissue reference level” refers to a level and/or amount of a marker, characteristic, and/or parameter in a white adipose tissue cell and/or tissue which has not been treated according to the methods described herein. In some embodiments, the white adipose tissue reference level of a marker can be the level of the marker in a white adipose tissue cell and/or tissue. In some embodiments, the white adipose tissue reference level can be the level in a sample of similar cell type, sample type, sample processing, and/or obtained from a subject of similar age, sex and other demographic parameters as the cell and/or tissue which is to be treated according to the methods described herein. Accordingly, in some embodiments, the white adipose tissue reference level of a brown adipose tissue-like phenotype marker can vary as demographic factors such as age, gender, genotype, environmental factors, and individual medical histories vary.

A brown adipose tissue-like phenotype can include an increase in a parameter selected from the group of RXR expression; RXR activity; UCP-1 expression; thermogenesis; and uncoupled mitochondrial respiration, as compared to an untreated WAT reference level. In some embodiments, an increase in a BAT-like phenotype can comprise an increase in a parameter selected from the group of RXR expression; RXR activity; UCP-1 expression; thermogenesis; and uncoupled mitochondrial respiration.

The term “obesity” refers to excess fat in the body. Obesity can be determined by any measure accepted and utilized by those of skill in the art. Currently, an accepted measure of obesity is body mass index (BMI), which is a measure of body weight in kilograms relative to the square of height in meters. Generally, for an adult over age 20, a BMI between about 18.5 and 24.9 is considered normal, a BMI between about 25.0 and 29.9 is considered overweight, a BMI at or above about 30.0 is considered obese, and a BMI at or above about 40 is considered morbidly obese. (See, e.g., Gallagher et al. (2000) Am J Clin Nutr 72:694-701.) These BMI ranges are based on the effect of body weight on increased risk for disease. Some common conditions related to high BMI and obesity include cardiovascular disease, high blood pressure (i.e., hypertension), osteoarthritis, cancer, and diabetes. Although BMI correlates with body fat, the relation between BMI and actual body fat differs with age and gender. For example, women are more likely to have a higher percent of body fat than men for the same BMI. The BMI threshold that separates normal, overweight, and obese can vary, e.g. with age, gender, ethnicity, fitness, and body type, amongst other factors. In some embodiments, a subject with obesity can be a subject with a body mass index of at 2 least about 25 kg/m prior to administration of a treatment as described herein. In some embodiments, a subject with obesity can be a subject with a body mass index of at least about 30 kg/m prior to administration of a treatment as described herein.

As used herein “excess adipose tissue” refers to an amount of adipose tissue present in the subject which is more than is desired. In some embodiments, excess adipose tissue can refer to adipose tissue which a medical practitioner has determined is contributing or can contribute to obesity and/or metabolic disease. In some embodiments, excess adipose tissue can refer to adipose tissue which a medical practitioner has determined to be more than the medically-recommended amount of adipose tissue for the particular subject and can be influenced by age, gender, ethnicity, fitness, and body type, amongst other factors. In some embodiments, excess adipose tissue can be adipose tissue that is determined to be more than aesthetically desirable.

As used herein, “diabetes” refers to diabetes mellitus, a metabolic disease characterized by a deficiency or absence of insulin secretion by the pancreas. As used throughout, “diabetes” includes Type 1, Type 2, Type 3, and Type 4 diabetes mellitus unless otherwise specified herein. The onset of diabetes is typically due to a combination of hereditary and environmental causes, resulting in abnormally high blood sugar levels (hyperglycemia). The two most common forms of diabetes are due to either a diminished production of insulin (in type 1), or diminished response by the body to insulin (in type 2 and gestational). Both lead to hyperglycemia, which largely causes the acute signs of diabetes: excessive urine production, resulting compensatory thirst and increased fluid intake, blurred vision, unexplained weight loss, lethargy, and changes in energy metabolism.

Diabetes can cause many complications. Acute complications (hypoglycemia, ketoacidosis, or nonketotic hyperosmolar coma) may occur if the disease is not adequately controlled. Serious long-term complications (i.e. chronic side effects) include cardiovascular disease (doubled risk), chronic renal failure, retinal damage (which can lead to blindness), nerve damage (of several kinds), and microvascular damage, which may cause impotence and poor wound healing. Poor healing of wounds, particularly of the feet, can lead to gangrene, and possibly to amputation. In some embodiments, the diabetes can be Type 2 diabetes. Type 2 diabetes (non-insulin-dependent diabetes mellitus (NIDDM), or adult-onset diabetes) is a metabolic disorder that is primarily characterized by insulin resistance (diminished response by the body to insulin), relative insulin deficiency, and hyperglycemia. In some embodiments, a subject can be pre-diabetic, which can be characterized, for example, as having elevated fasting blood sugar or elevated post-prandial blood sugar.

As used herein, “cardiovascular disease” refers to various clinical diseases, disorders or conditions involving the heart, blood vessels or circulation. The diseases, disorders or conditions may be due to atherosclerotic impairment of coronary, cerebral or peripheral arteries. Cardiovascular disease includes, but is not limited to, coronary artery disease, peripheral vascular disease, hypertension, myocardial infarction, heart failure, stroke, and angina.

The following non-limiting examples illustrate some aspects of the invention.

### EXAMPLE 1

### Materials and Methods

This Example describes some of the materials and methods used in the development of the invention.

### Cell Culture

C2C12, 3T3-L1 and C3H10T1/2 cells were purchased from ATCC. Primary brown pre-adipocytes were kindly donated by Prof. C. Ronald Kahn (Harvard University). Primary myoblasts were purified from mouse limb muscles and cultured in growth medium (F10+20% FBS+5 ng/ml bFGF) in Matrigel-coated plates (Xiao et al., Cell Res 21, 350-364 (2011)). Adipocyte differentiation was induced by treating cells for 2 days in basal adipogenesis medium (AM) (850 nM insulin, 1 nM T3, 0.5 mM isobuylmethylxanthine, 125 1-μM indomethacin, 5 μM dexamethasone in10% FBS DMEM). Then cells were switched to 10% FBS DMEM containing 850 nM insulin and 1 nM T3 for another 4-6 days. To stimulate thermogenesis, cells were stimulated with 10 μM forskolin for 4 h.

### Constructs and Chemicals

Mouse retinoid X receptor-alpha (RXRα) and retinoid X receptor-gamma (RXRγ) plasmids were from Dr. Ronald Evans (Salk Institute) and mouse RXRβ plasmid was purchased from Origene. These three RXR fragments were subcloned into pMXs vectors to generate a retrovirus construct (Yamanaka lab, Gladstone Institutes). Non-target shRNA(shNT), shRXRα, shRXRβ, and shRXRγ lentivirus constructs were purchased from Sigma-Aldrich. Retrovirus and lentivirus constructs were packaged in Plat E and 293T cells, respectively. Bexarotene, HX531, rosiglitazone and cPGI2 (carbaprostacyclin) was purchased from Thermo Fisher Scientific, Tocris Bioscience, Sigma Aldrich and Cayman Chemical Co., respectively.

### Real-Time PCR

Total RNA from cell lines were purified using Qiagen kit, and total RNA from mouse tissues was extracted using Trizol. Complementary DNA was prepared from total RNA with the iScript DNA synthesis kit (Bio-Rad). Quantitative PCR reactions contained SYBR-Green fluorescent dye (ABI). RT-PCR was also used to confirm some gene expression. Relative mRNA expression was determined by the ΔΔ-Ct method with GAPDH as an endogenous control.

### Affymetrix Microarray Analysis

Mouse genome-wide gene expression analyses were performed using Affymetrix Mouse Gene 1.0 ST Array. Total RNA samples were purified from C2C12 cells after a 2-day basal adipogenesis medium treatment without bexarotene (control) or with bexarotene (Bex) with RNeasy mini plus kit from Qiagen. Microarray analyses were performed in duplicate from independent biologic samples, according to the standard Affymetrix Genechip protocol. Data were normalized and analyzed using Affymetrix Expression Console and Transcriptome Analysis Console (TAC) software. A gene was regarded as significantly changed if the P-value was <0.05 and fold was greater than 1.5 or less than −1.5. The differential expression gene list was further analyzed in DAVID GO functional annotation and GO-Elite (Zambon et al., Bioinformatics 28: 2209-2210 (2012)).

### Animal Work

C57BL/6N mice were fed ad libitum a standard laboratory chow diet (LabDiet 5053, LabDiet, Purina Mills, Richmond, Ind.). Animals were housed under 12-hour light-dark cycles with controlled temperature (23±1° C.). 8-week old male mice were treated with bexarotene (50 mg/kg/day) or saline by daily oral gavage for 4 weeks. All animal experiments were conducted in accordance with the institutional Guidelines for humane treatment of laboratory animals of HKU Animal Care and Use Committee.

### Indirect Calorimetry

Whole-body oxygen consumption was measured using an open circuit indirect calorimetry system with automatic temperature and light controls (Columbus Instruments). Mice had access ad libitum to chow and water in respiration chambers, and data were recorded for a 48 h period before acclimatized for 24 h.

### Statistical Analysis

Statistical significance in gene expression between the control and the bexarotene-treated group was determined by analysis of Student's t test unless otherwise specified.

### EXAMPLE 2

### Bexarotene Selectively Promotes Brown Adipogenesis but Inhibits White Adipocyte Differentiation

This Example describes identification of small molecules that induce brown adipogenesis.

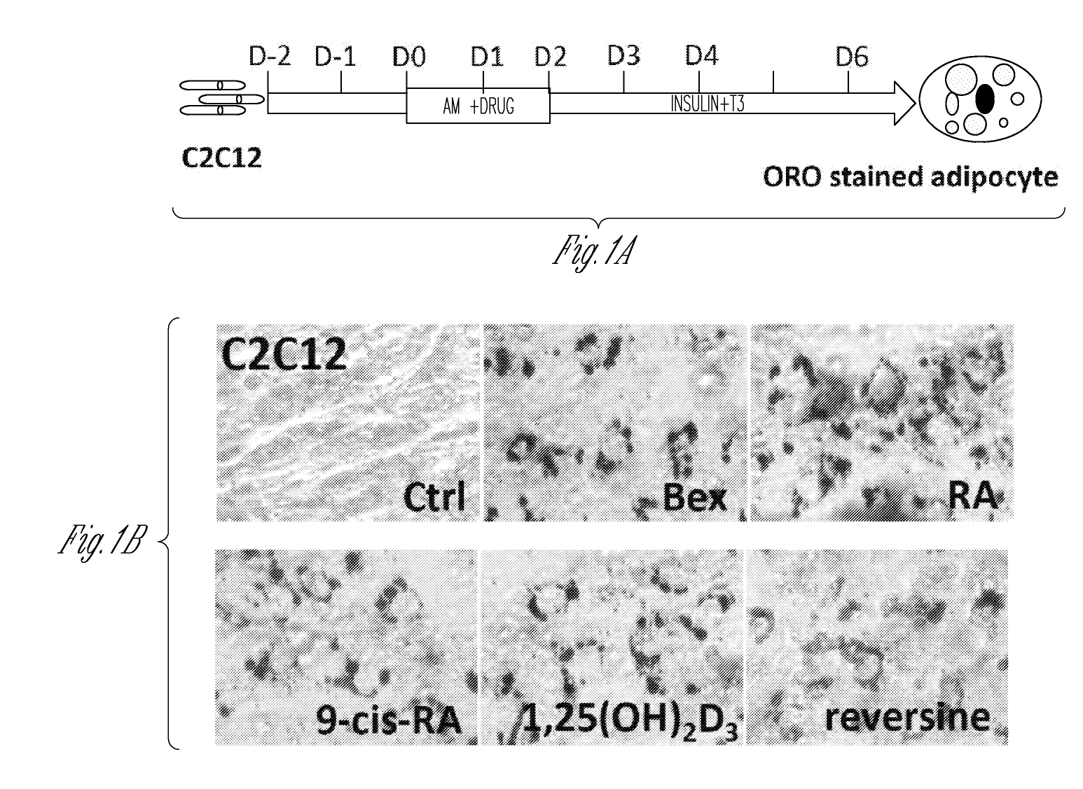

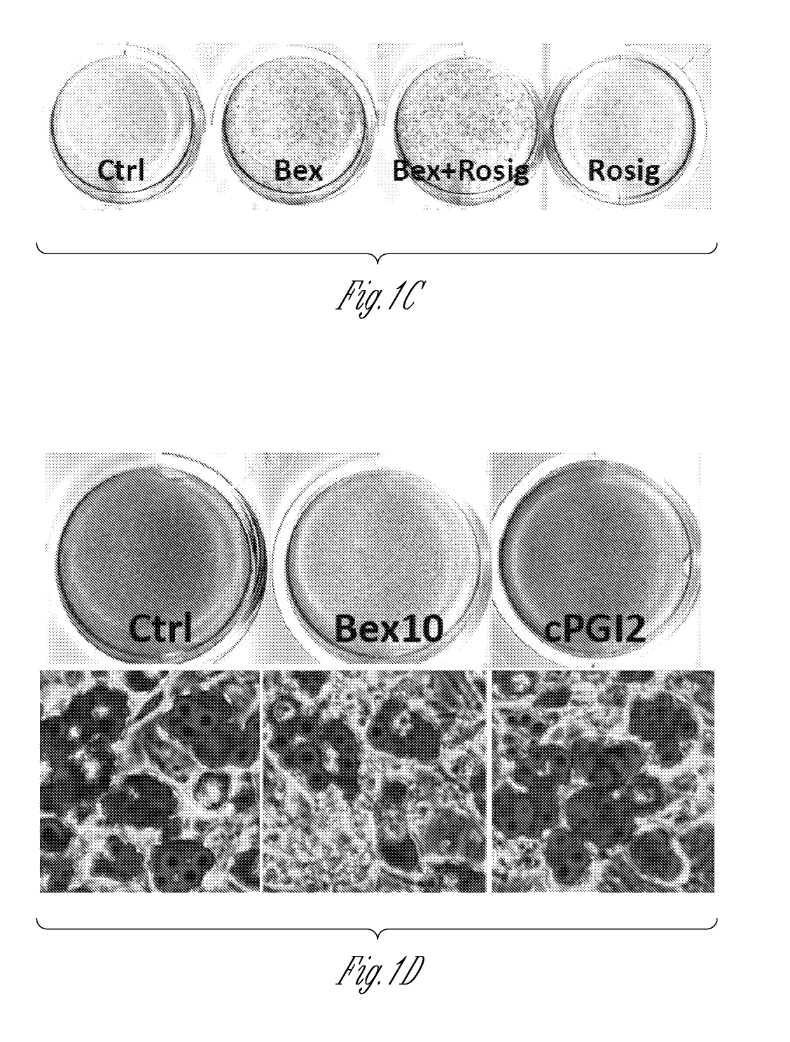

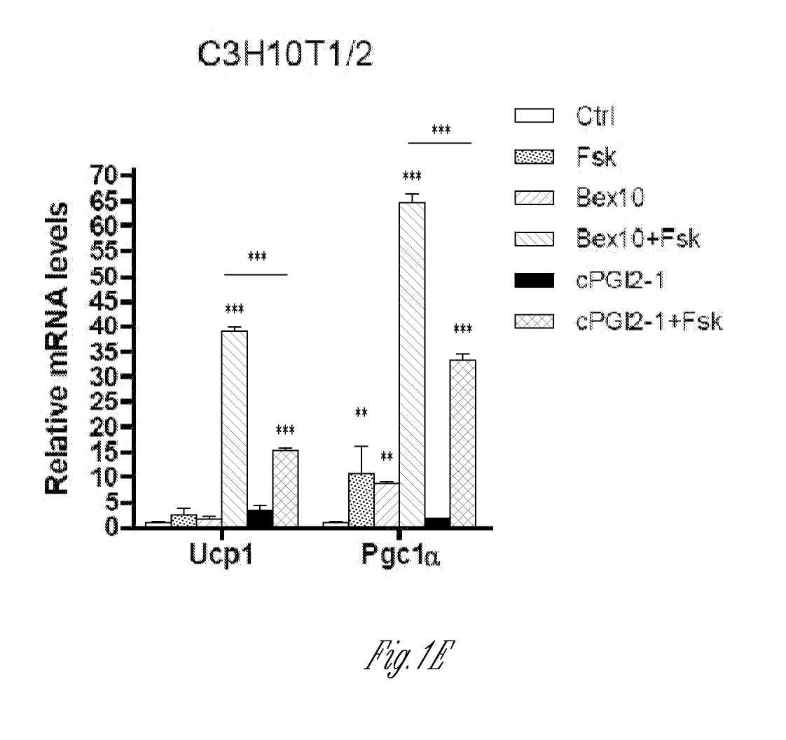

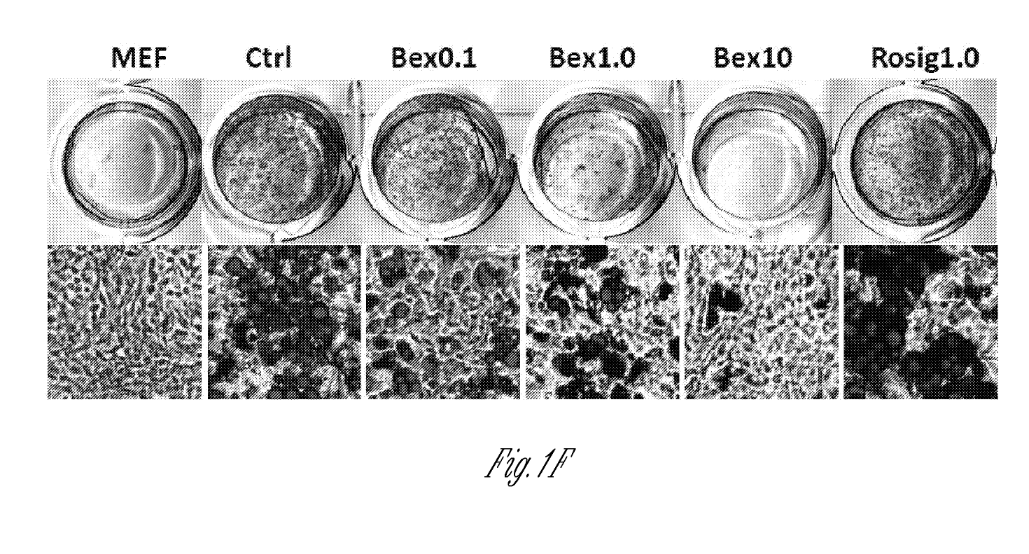

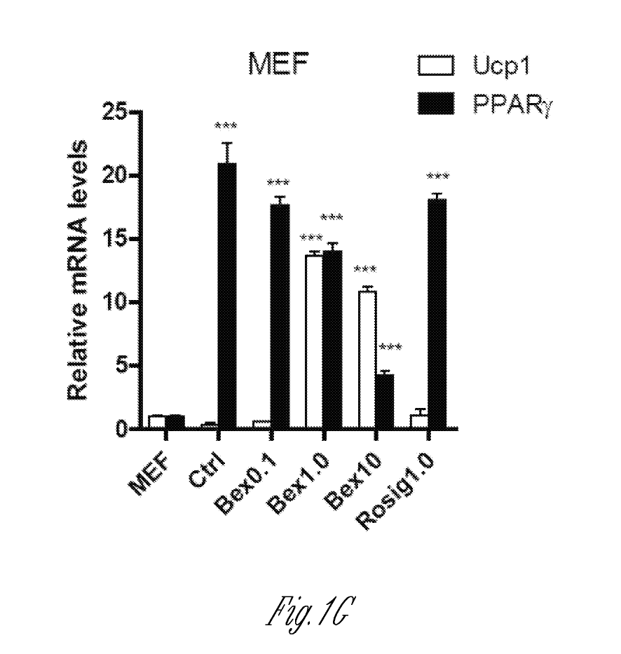

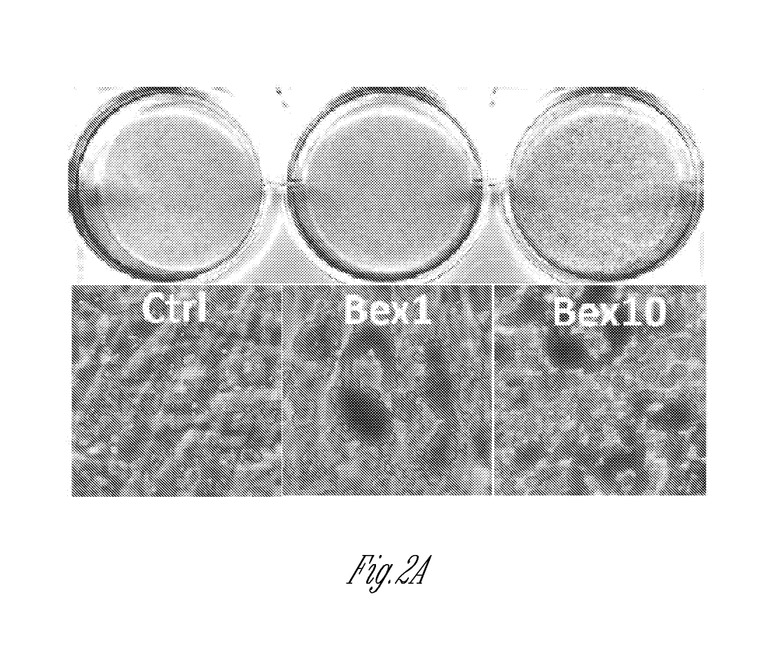

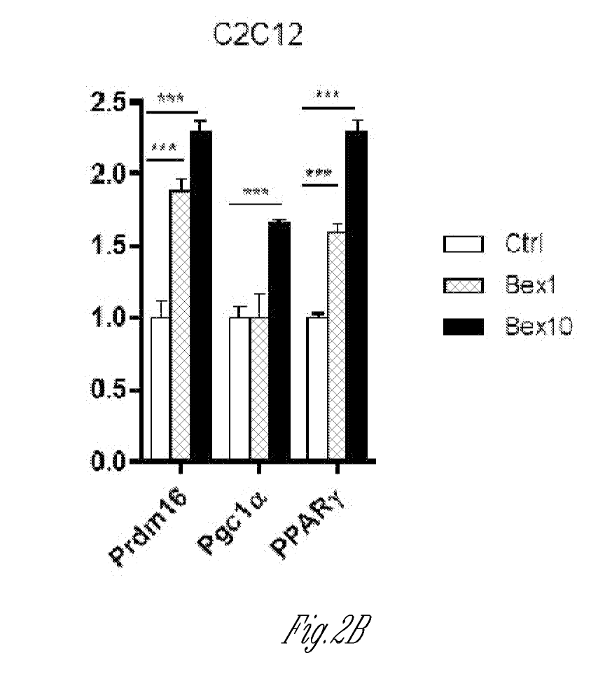

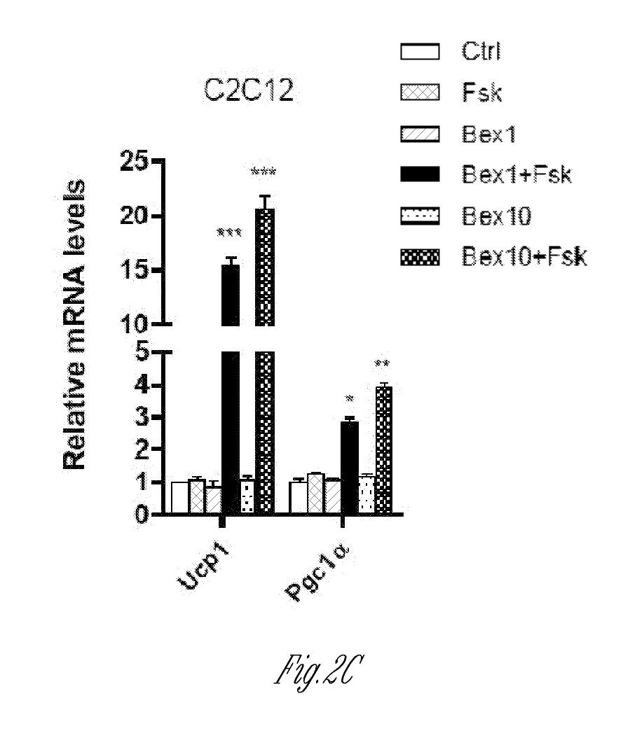

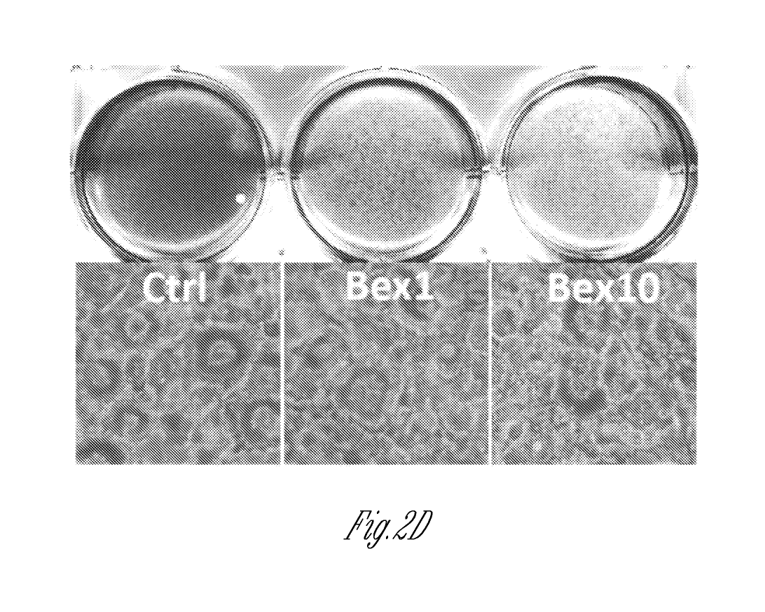

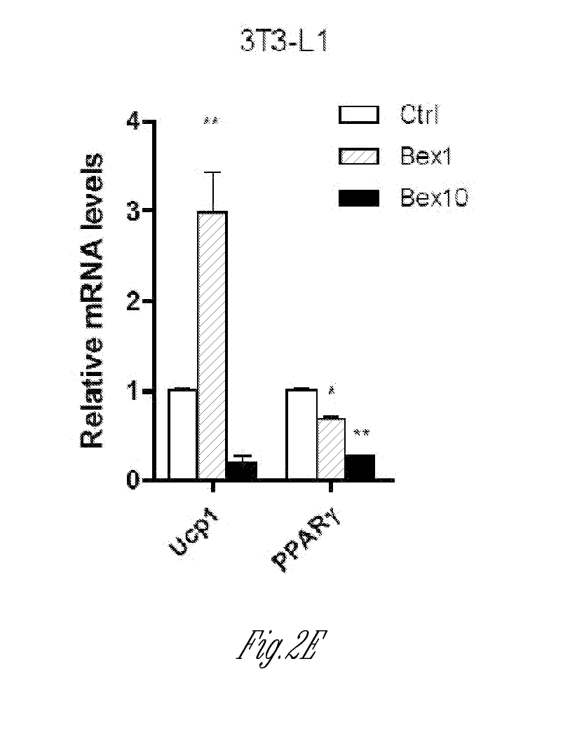

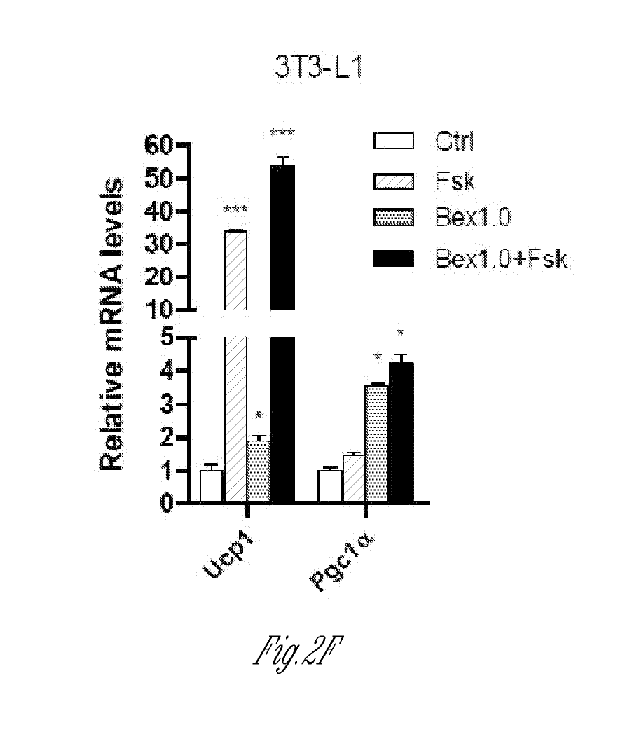

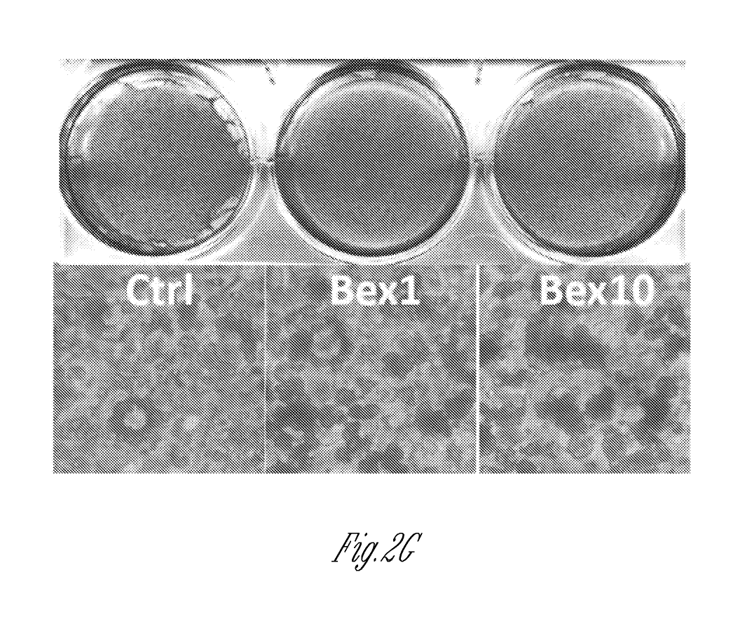

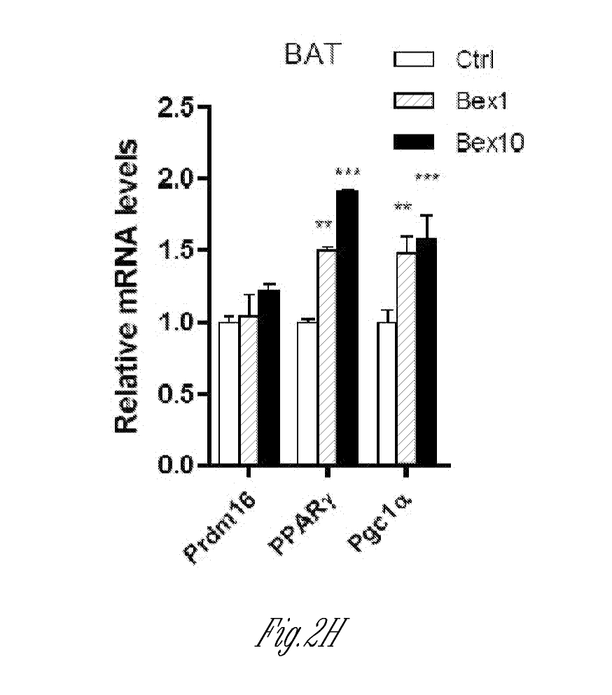

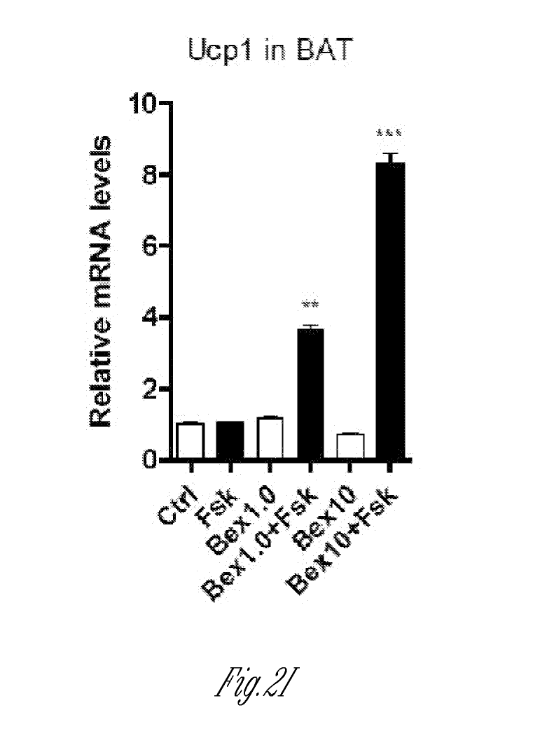

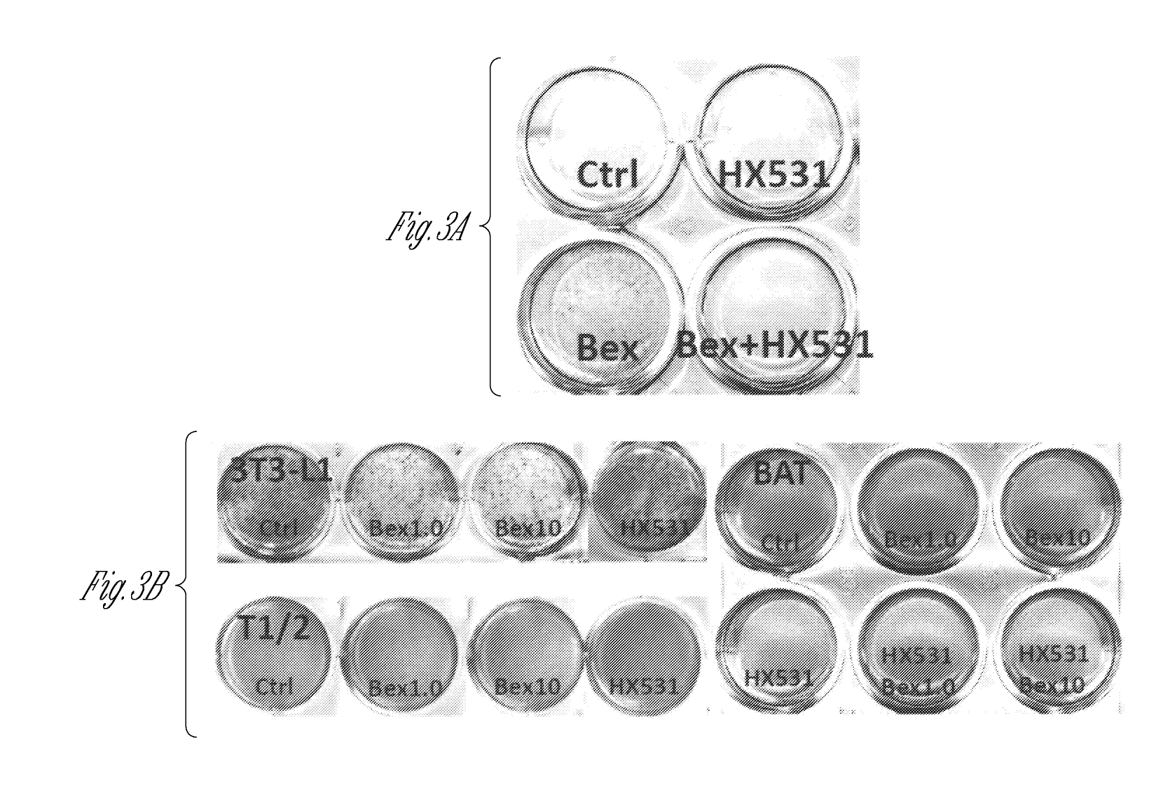

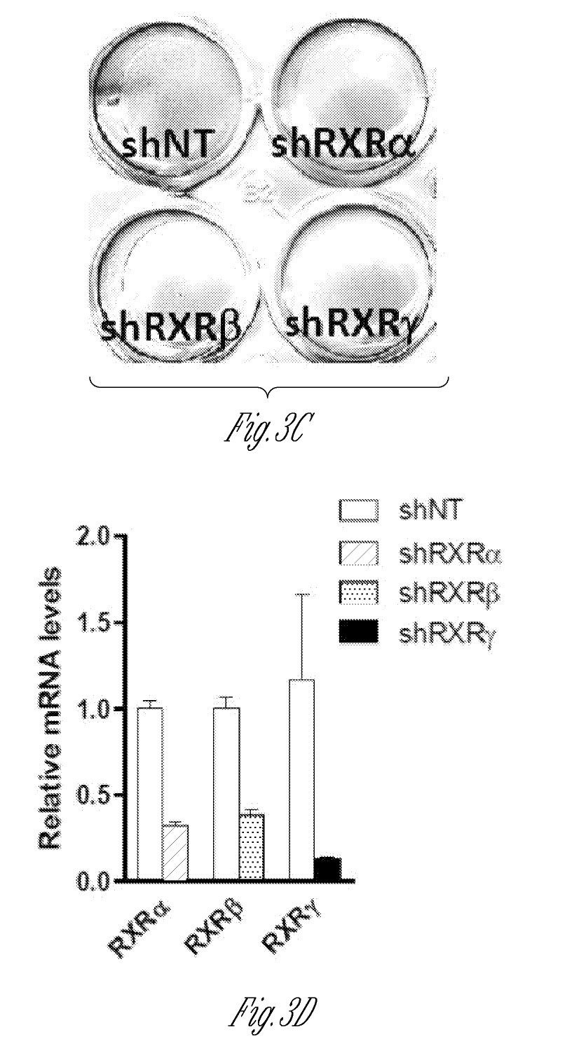

To identify small molecules that mimic the BAT-inducing activity of Prdm16, a high-throughput phenotypic screen of 20,000 compounds was performed using the C2C12 myoblast cell line with lipid droplets stained by Oil-red-O (ORO) as readout (FIG. 1A). Among several primary hits, bexarotene led to the appearance of lipid droplets in C2C12 cells (FIG. 1B). In the basal adipogenesis medium (AM), no adipocyte-like cells could be induced from C2C12 myoblasts. In contrast, addition of bexarotene for 2 days dramatically initiated adipogenic reprogramming in C2C12 cells in a dose-dependent manner (FIG. 2A). Gene expression analysis showed that bexarotene significantly induced the expression of adipogenesis maker PPARγ, and the brown adipocyte-specific genes Prdm16 and Pgc1α (FIGS. 2B-2C). Furthermore, these BAT-like adipocytes readily responded to forskolin (Fsk), which induces expression of thermogenic genes Ucp1 and Pgc1α (FIG. 2C). Moreover, the effect of bexarotene could be further enhanced by the PPARγ agonist rosigliotazone (Rosig), even though rosigliotazone itself could not induce adipocyte formation from myoblasts (FIG. 1C). These data show that bexarotene elicited a BAT-like reprogramming in C2C12 myoblasts.

Tests were then performed to determine whether bexarotene similarly induced BAT reprogramming in other non-BAT cell types. PRDM16 reprograms the white adipogenic lineage (e.g., 3T3-L1 white pre-adipocytes) toward the brown adipocyte lineage by activating the BAT program and simultaneously suppressing the white adipose tissue program (Seale et al., Cell Metab 6: 38-54 (2007)). Thus, 3T3-L1 white pre-adipocytes were treated with bexarotene under the brown adipogenic differentiation conditions. Remarkably, bexarotene significantly suppressed the typical white adipose tissue differentiation along with the adipogenic gene PPARγ in 3T3-L1 cells (FIG. 2E-2G) while BAT features, such as small lipid droplets and Ucp1 expression, were induced (FIG. 2D-2F). Furthermore, cells treated with bexarotene exhibited more robust response to forskolin stimulation (FIG. 2E-2F), indicating that bexarotene shifted 3T3-LI cells from the white towards a brown phenotype.

C3H10T1/2 mesenchymal cells and primary mouse embryonic fibroblast (MEF) cells were examined to evaluate the effects of bexarotene on uncommitted mesenchymal precursor cells (which have a multi-lineage differentiation potential, including adipogenic, osteogenic, chondrogenic, and myogenic lineages). These two types of cells readily differentiate into white adipocytes and respond to BAT inducers, including Prdm16 overexpression and BMP7 treatment, to exhibit BAT phenotypes (Seale et al., 2007; Tseng et al., 2008). Consistently and significantly, bexarotene treatment of C3H10T1/2 cells for even 2 days at the beginning of adipogenic induction induced the brown phenotype, marked by substantially increased levels of thermogenic gene expression to forskolin stimulation (FIG. 1D-1E). Notably, the effect of bexarotene on inducing Ucp1 and Pgc1α expression was much greater than that of the COX-2 agonist cPGI2 (FIG. 1D), which has been reported by Vegiopoulos et al. to promote a brown phenotype (Vegiopoulos et al., Science 328, 1158-1161 (2010)). Similarly, bexarotene induced a strong brown phenotype in primary mouse embryonic fibroblast (MEF) cells while apparently suppressing the white adipogenic phenotype (FIG. 1F-1G). Ucp1 expression was induced in a dose-dependent manner, even without forskolin stimulation (FIG. 1G). In contrast, the widely used white adipogenic inducer rosiglitazone did not induce browning (FIG. 1F-1G).

The effect of bexarotene on the differentiation of primary brown pre-adipocytes (pre-BAT) was further examined to confirm that bexarotene selectively suppresses white adipogenesis. Primary brown pre-adipocytes cells efficiently differentiated into brown adipose tissue in the conventional adipogenesis medium. But treatment with bexarotene significantly enhanced the differentiation efficiency of primary brown pre-adipocytes as show by the increased number of brown adipose tissue adipocytes observed, and by the higher levels of expression of PPARγ and several BAT-associated genes, including Prdm16 and Pgc1α (FIG. 2H). In addition, bexarotene potentiated the induction of Ucp1 and Pgc1α expression in response to forskolin in differentiated BAT adipocytes (FIG. 2I). These data demonstrate that bexarotene preferentially promotes brown adipogenic reprogramming in multiple cell types.

### Bexarotene Induces Brown Adipogenic Reprogramming Via Activation of RXRα and RXRγ

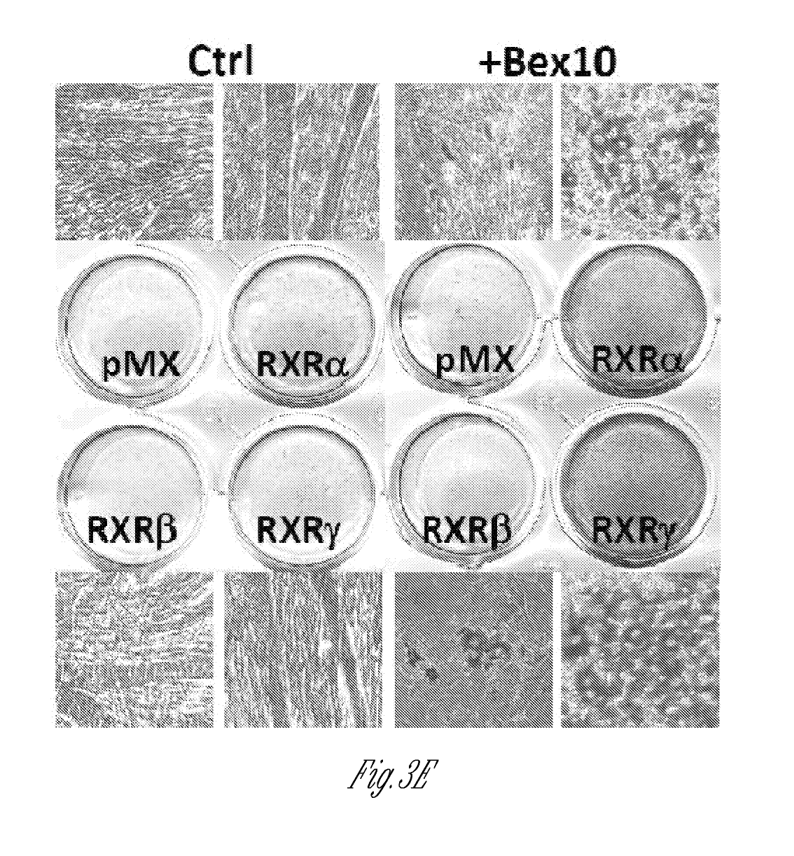

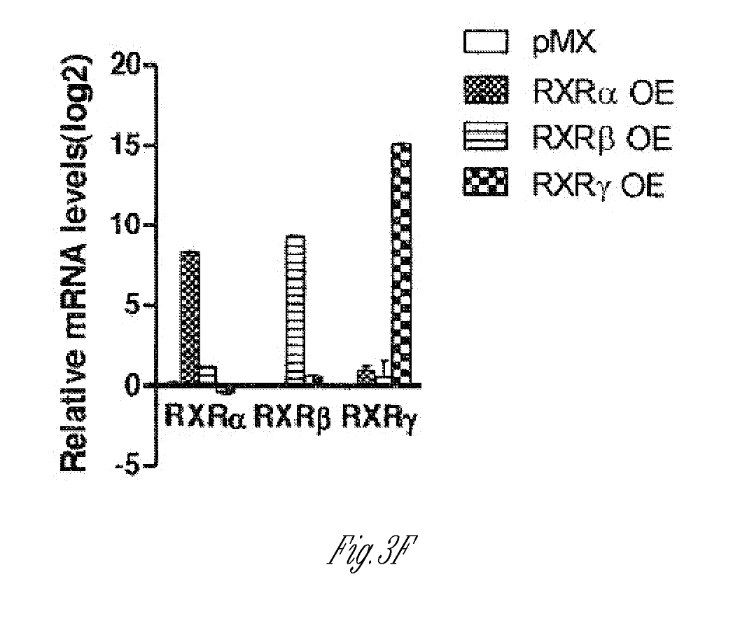

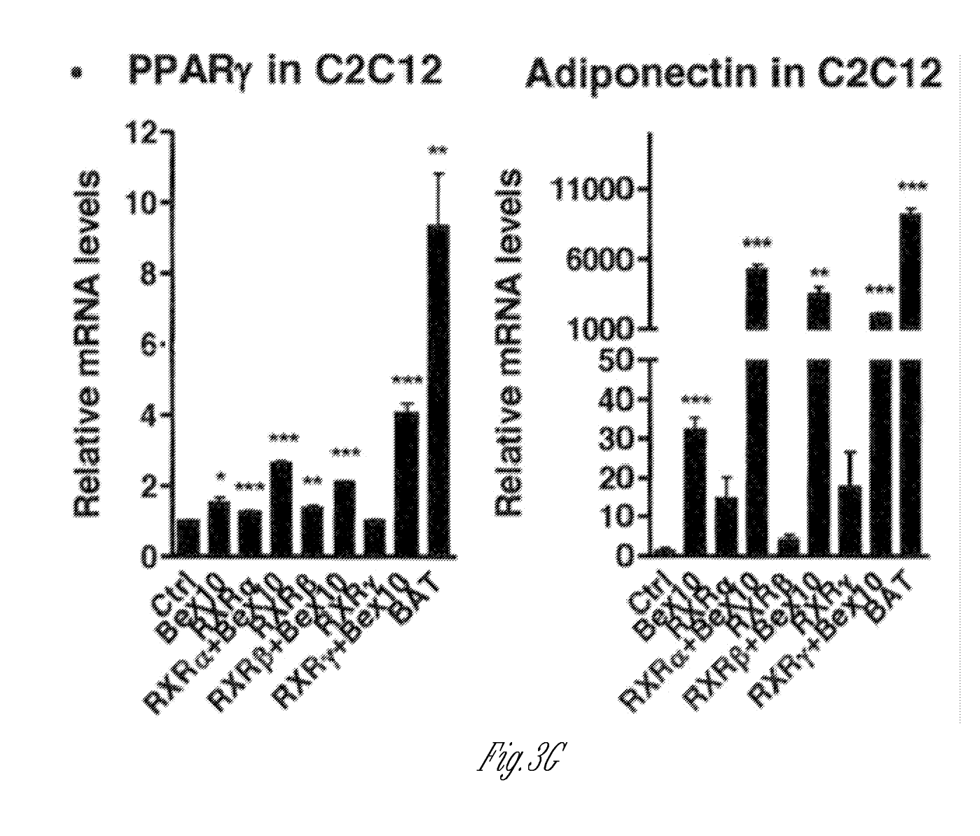

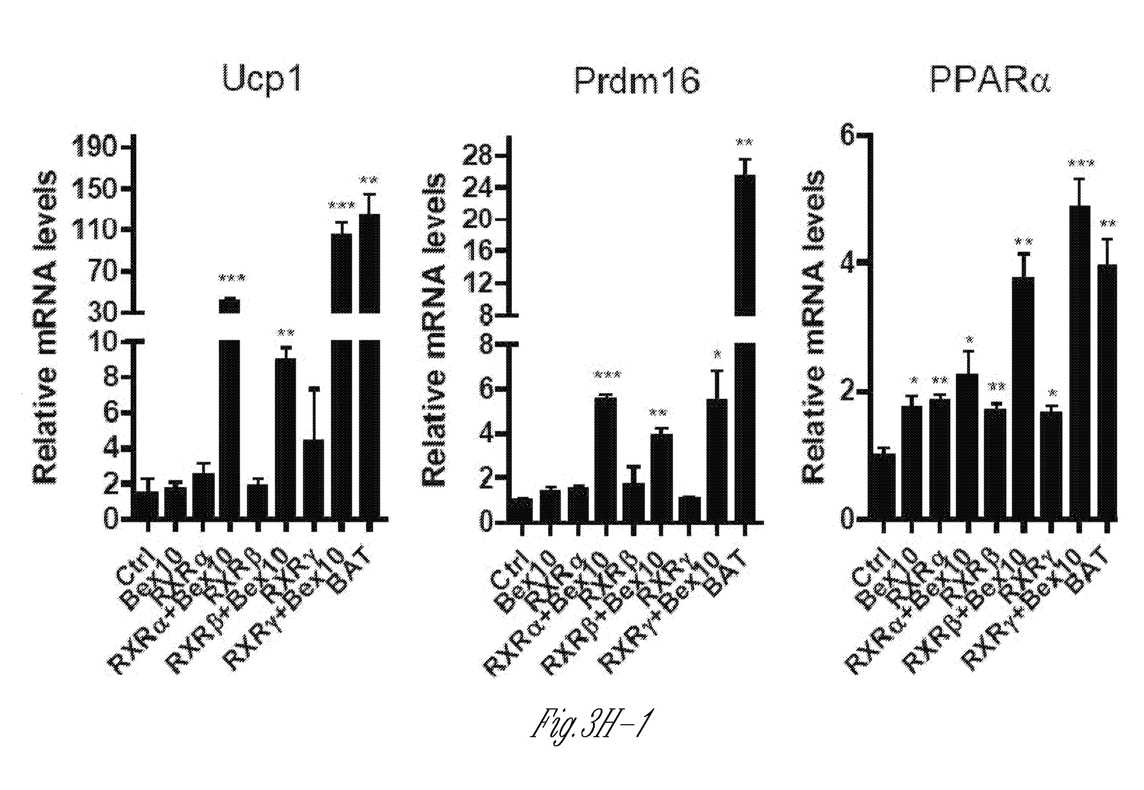

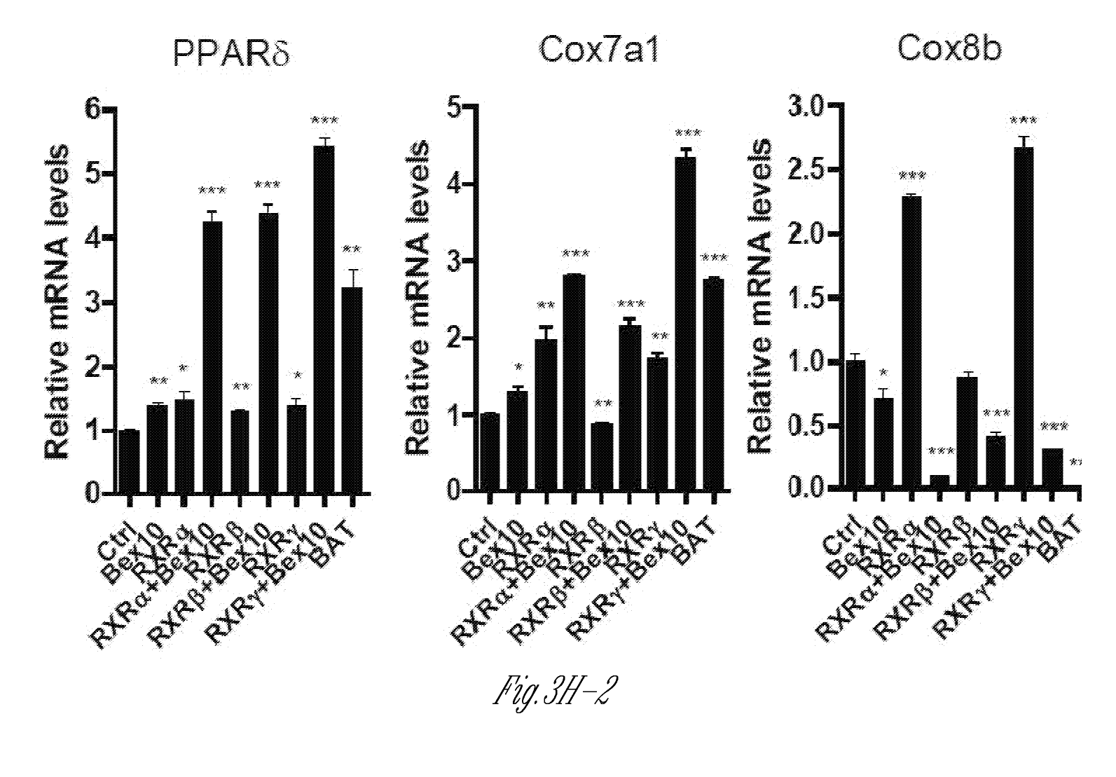

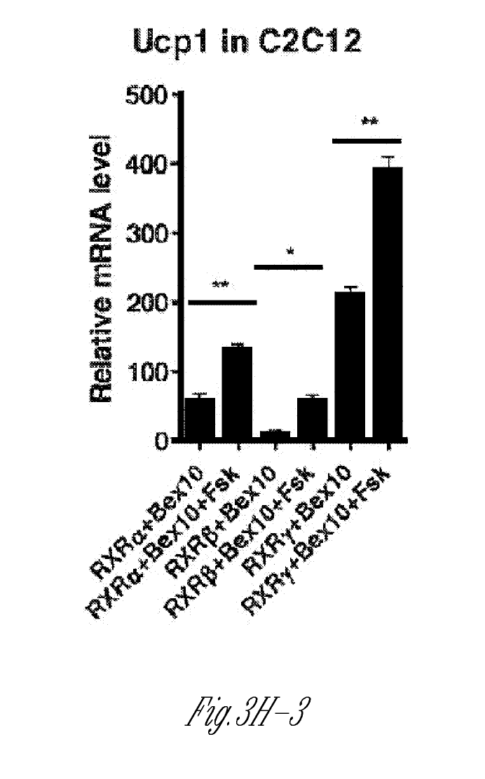

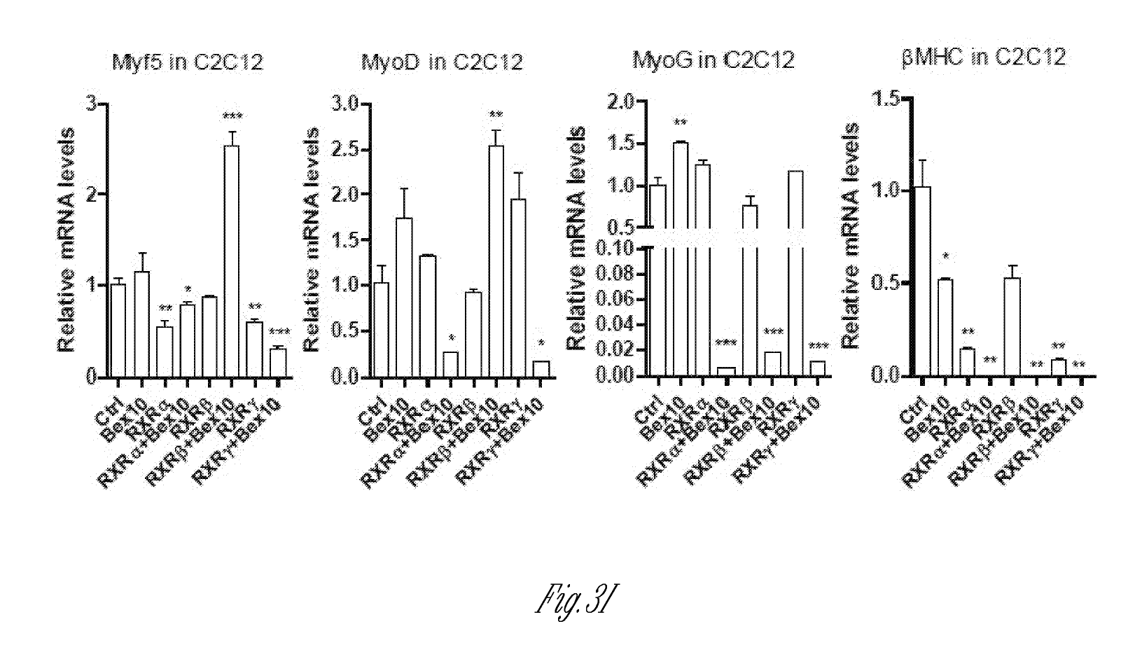

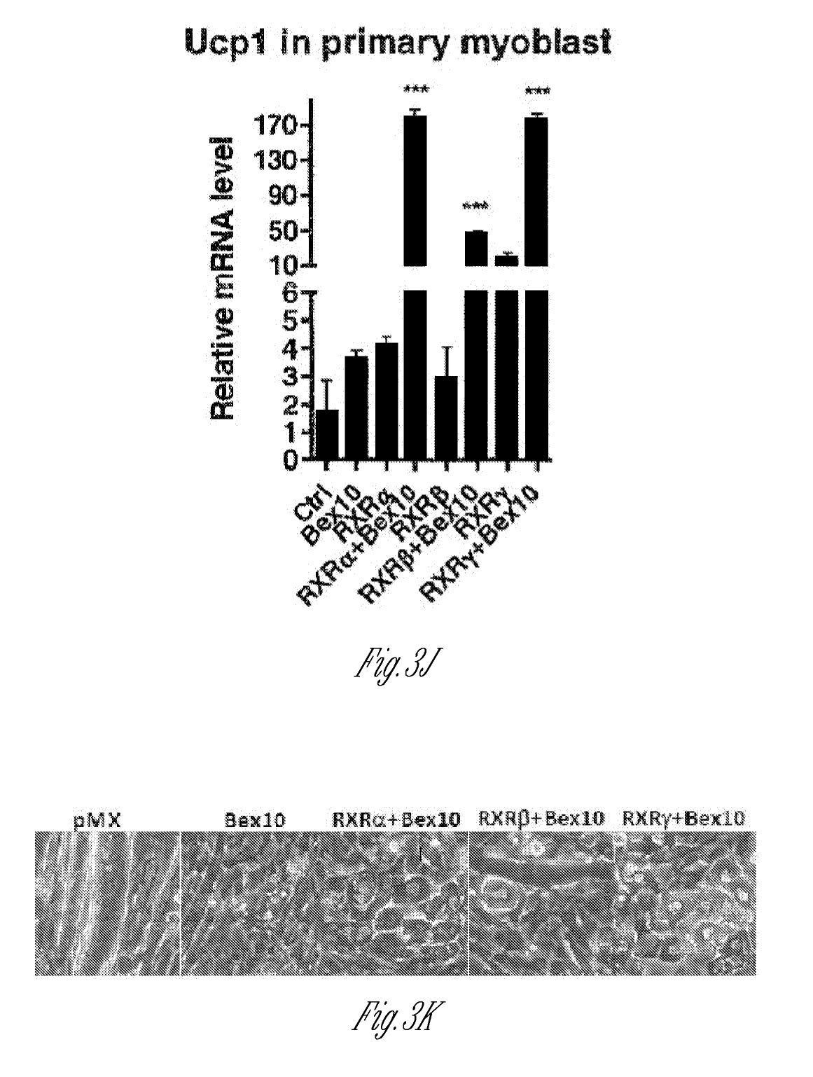

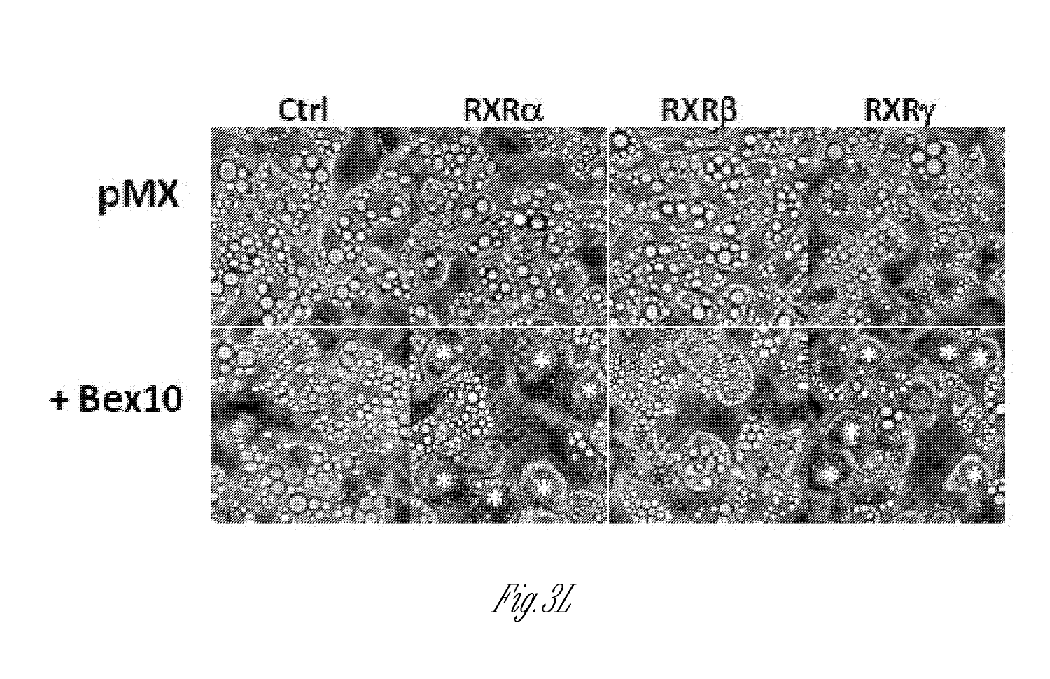

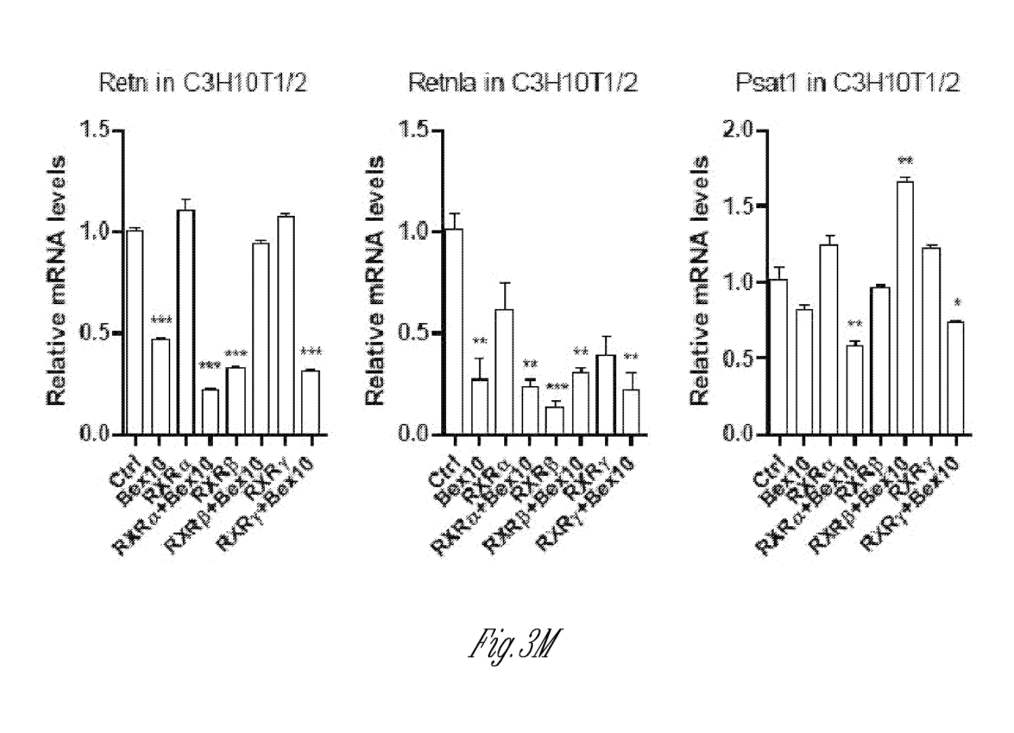

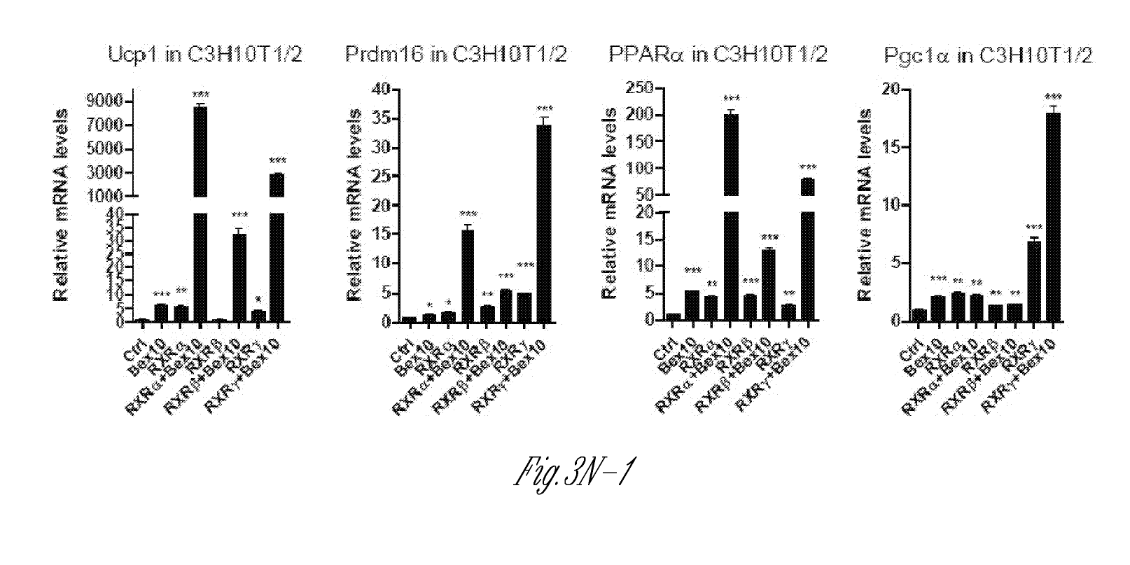

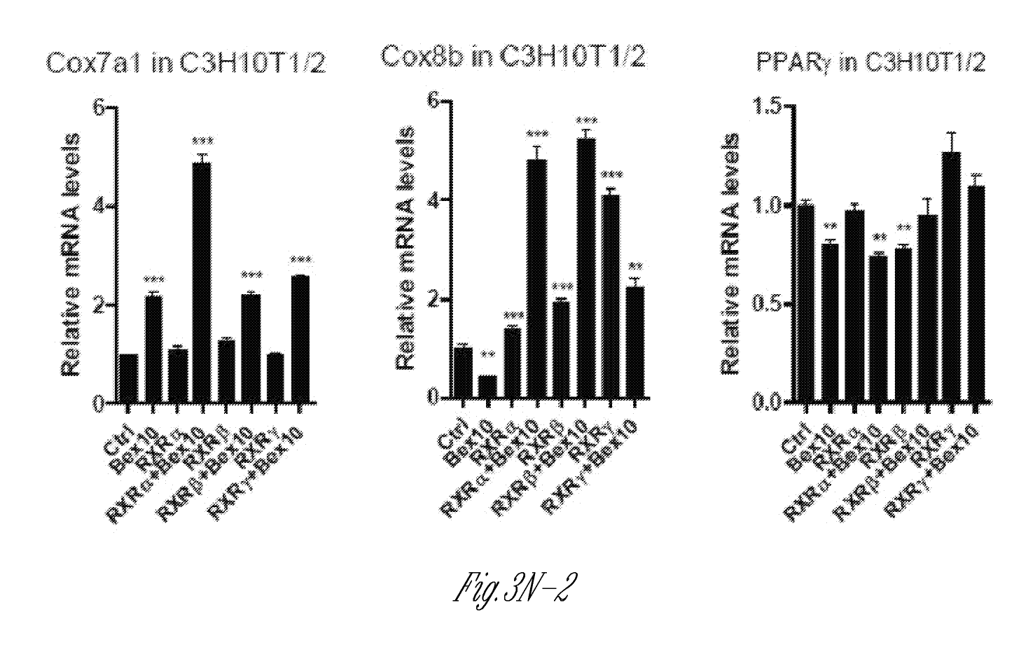

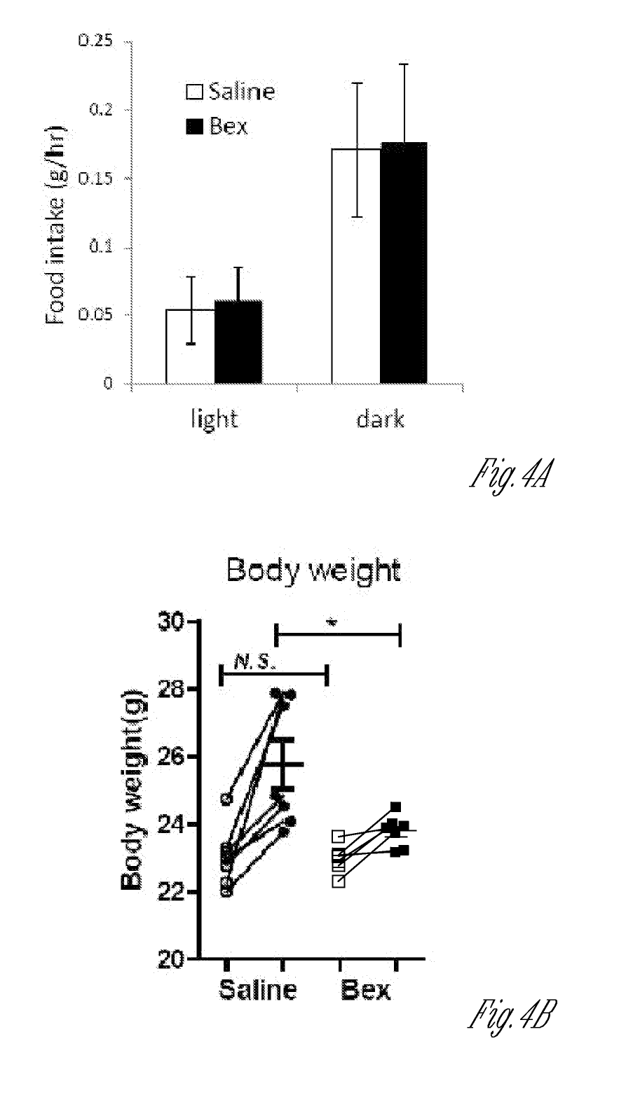

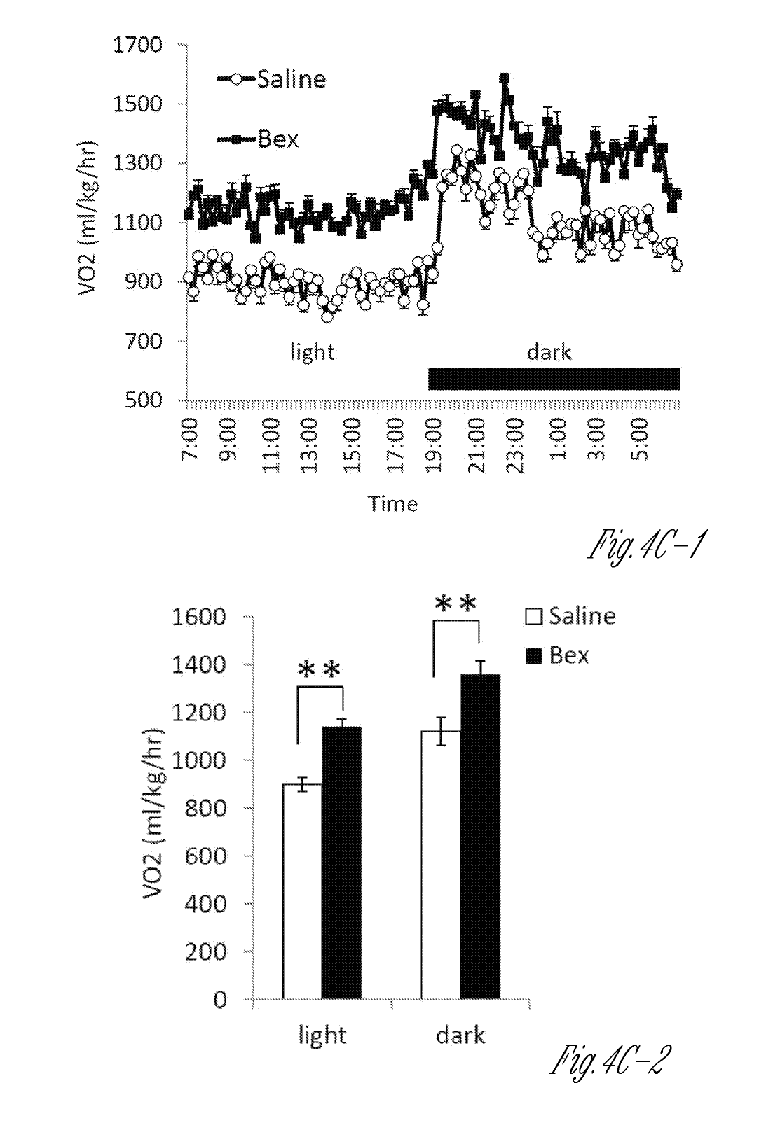

Bexarotene was originally developed as a selective retinoid X receptor (RXR) agonist. To characterize the mechanism by which bexarotene induces the brown adipogenic reprogramming, the inventors first determined whether RXR was required for its effect. As shown in FIGS. 3A and 3B, the selective RXR antagonist, HX531, completely abolished bexarotene-induced brown adipogenic induction in C2C12 cells.

To further confirm the relationship between bexarotene and RXR, the inventors knocked down RXR expression by introducing specific shRNAs that target each RXR subtype. As shown in FIG. 3C-3D, these small hairpin RNAs largely abolished bexarotene-induced adipogenic reprogramming in C2C12 cells compared to a control that did not express a RXR-specific small hairpin RNA (shNT). More importantly, HX531 had the opposite effect of bexarotene. While HX531 promoted white adipose tissue adipogenesis in both 3T3-L1 and C3H10T1/2 cells, it also reduced Bex-induced BAT differentiation. These data confirm that bexarotene acts through RXR activation and possesses opposite effects on adipogenesis in white adipose tissues (WAT) and brown adipose tissues (BAT).

To further characterize the role of retinoid receptors, the RXRα, RXRβ and RXRγ subtype receptors were overexpressed in C2C12 cells. RXRα, RXRβ or RXRγ overexpression alone was not sufficient to induce brown adipose tissue adipogenesis in C2C12 cells. However, upon addition of bexarotene, nearly all cells that over-expressed RXRα or RXRγ were reprogrammed to brown adipose-like cells with multilocular lipid droplets (FIG. 3E-3F). However, RXRβ overexpression did not significantly increase brown adipose tissue-induction efficiency in C2C12 cells even in the presence of bexarotene (FIG. 3E-3F). Gene expression analysis demonstrated that these reprogrammed cells expressed typical brown adipose tissue genes at levels comparable to those in differentiated brown adipocytes, including the general adipogenic genes PPARγ and adiponectin (FIG. 3G), and brown adipose tissue-specific genes, such as Ucp1, Prdm16, PPARα, PPARδ, Cox7a1 and Cox8b (FIGS. 3H-1 and 3H-2). In addition, bexarotene/RXR-induced brown adipose tissue-like cells robustly responded to forskolin to induce higher Ucp1 expression (FIG. 3H-3). On the other hand, myogenic genes, including Myf5, MyoD, and MyoG, and the late myogenic maker MHCβ were dramatically attenuated in these cells (FIG. 3I). Results similar to those with bexarotene/RXR in C2C12 cells were observed in primary myoblasts isolated from mouse skeletal muscle (FIGS. 3J and 3K). Consistent brown adipose tissue induction by bexarotene/RXRα and bexarotene/RXRγ were also observed in C3H10T1/2 cells (FIG. 3L). Specifically, bexarotene/RXRα and bexarotene/RXRγ suppressed white adipose tissue-specific genes, such as resistin (Retn), resistin-like alpha (retn1α), and phosphoserine aminotransferase 1 (Psat1) (FIG. 3M) in C3H10T1/2 cells under adipogenic induction, while they induced brown adipose tissue characteristic small multilocular lipid droplets (FIG. 3L) and expression of brown adipose tissue-specific genes, including Ucp1, Prdm16, PPARα, Pgc1α, Cox7a1, and Cox8b (FIGS. 3N-1 and 3N-2). The above results show that RXRα/RXRγ activation is required for brown adipose tissue induction in myoblasts and white adipocyte cell line.

### Bexarotene Improves Brown Adipose Tissue Mass and Function In Vivo

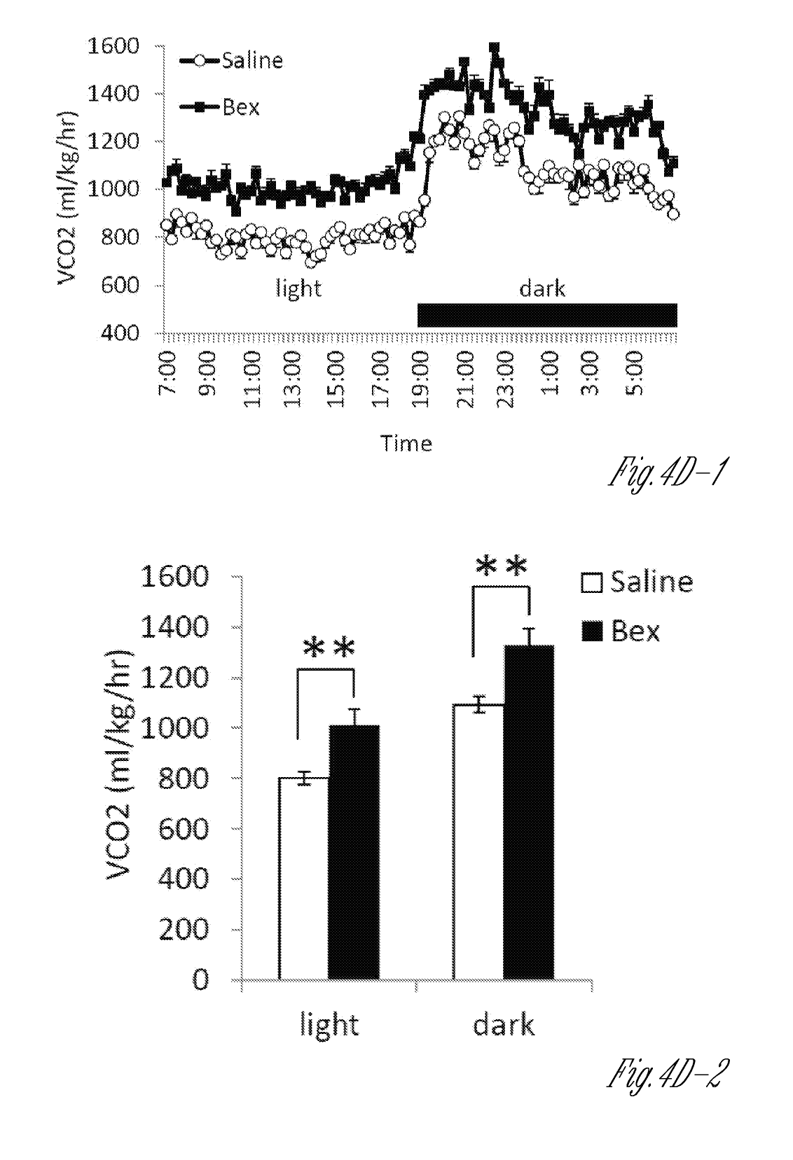

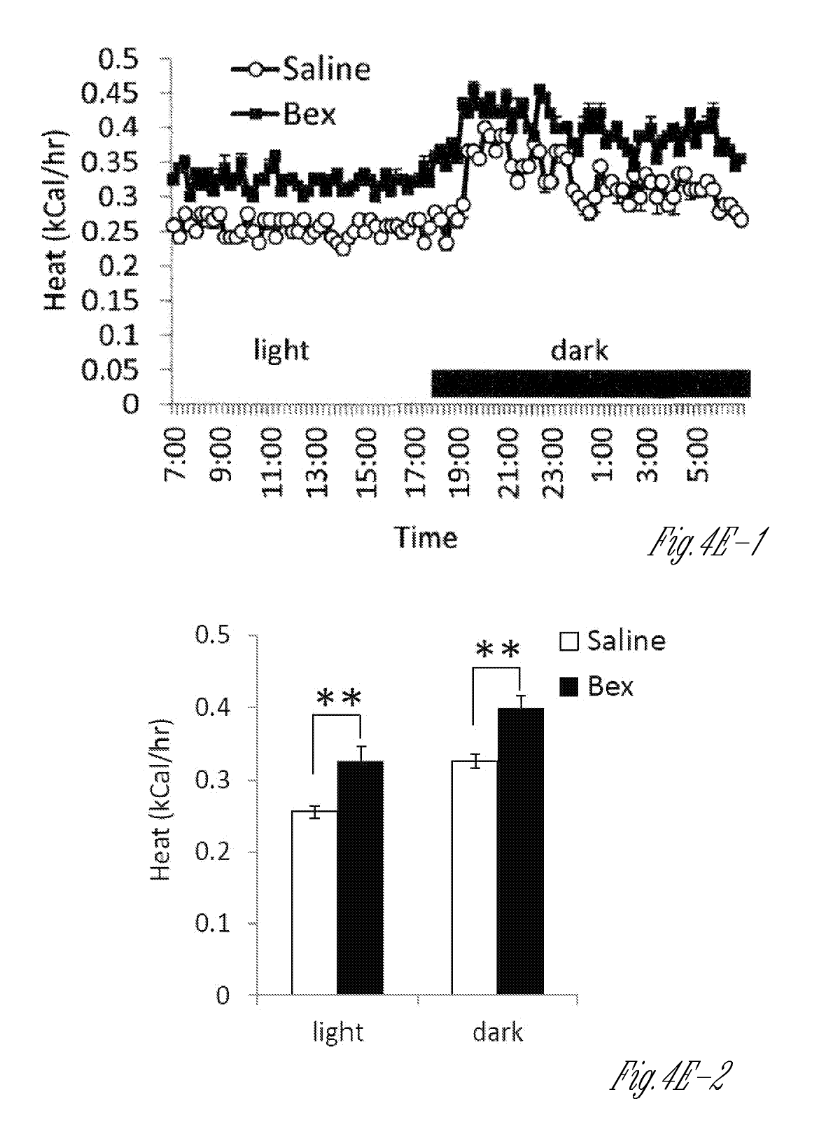

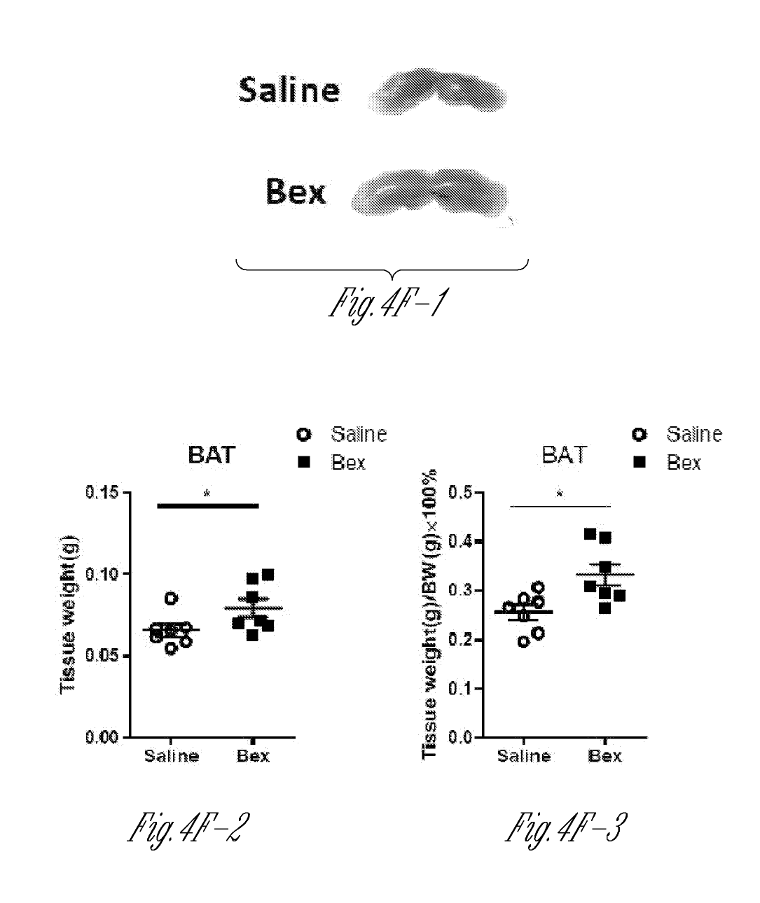

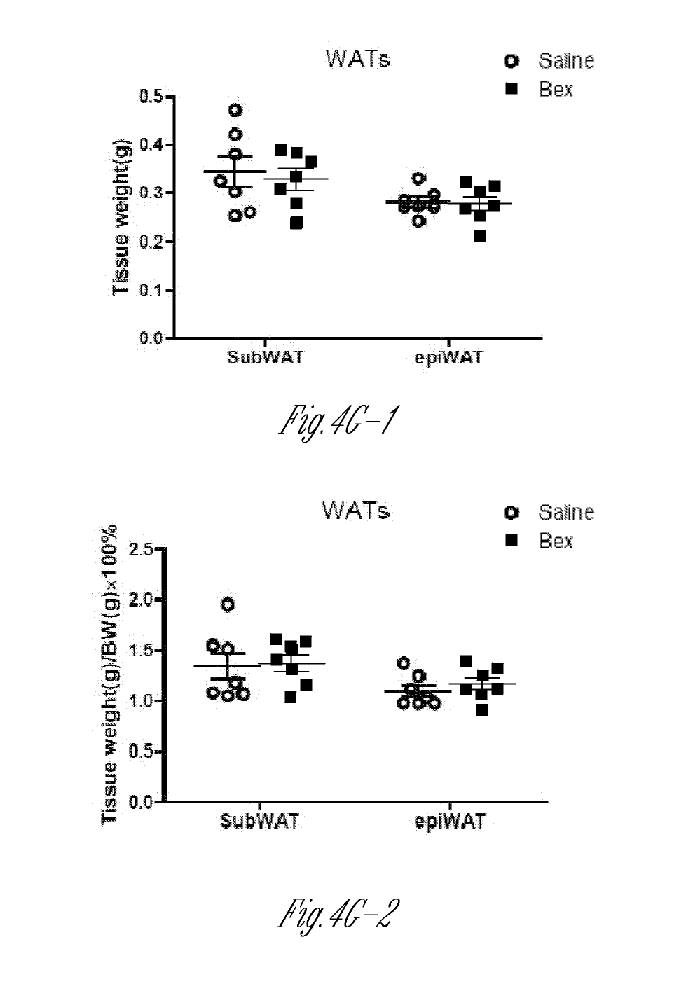

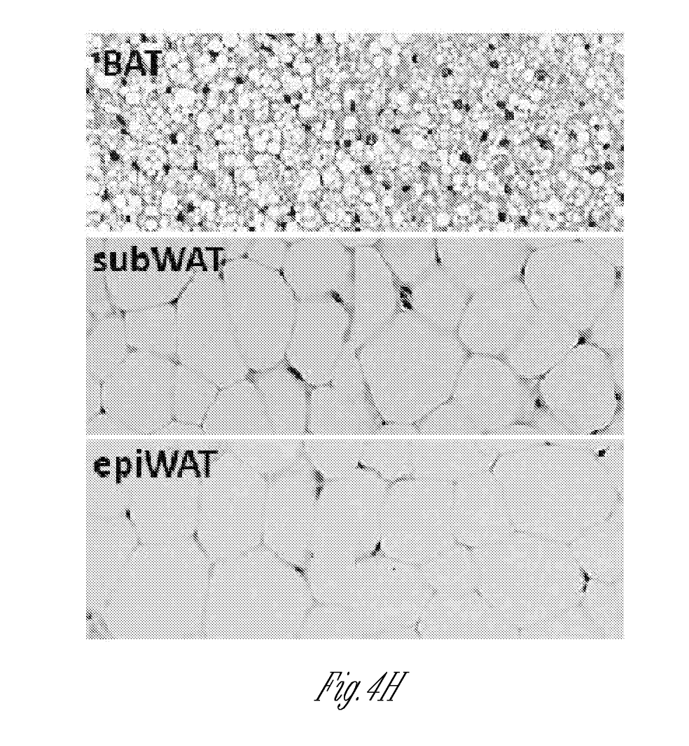

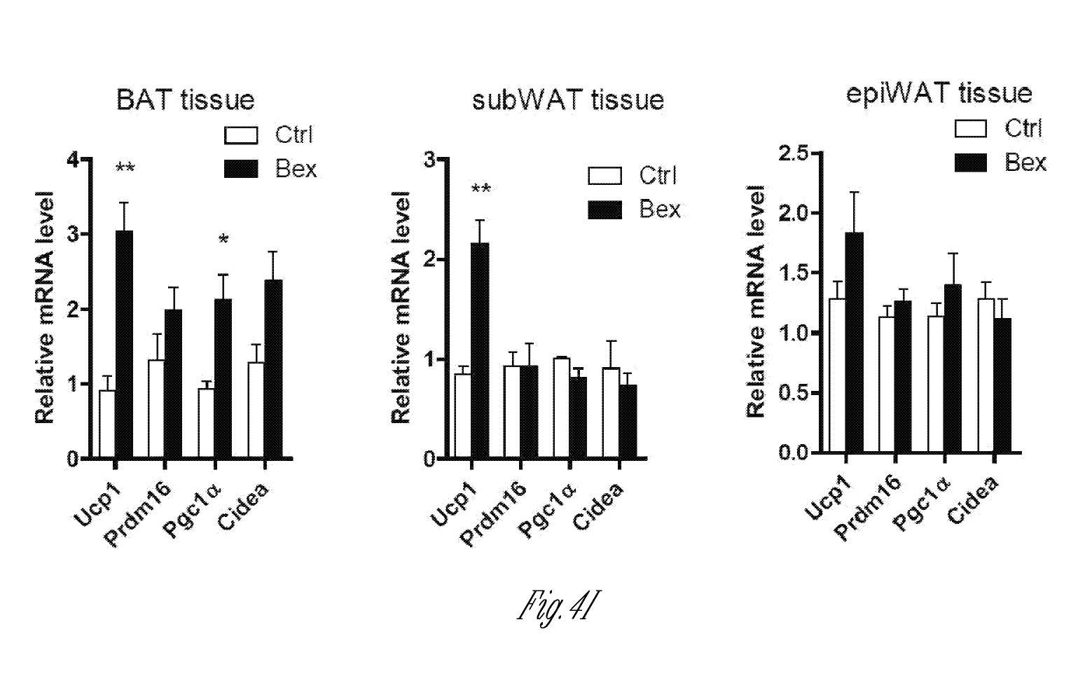

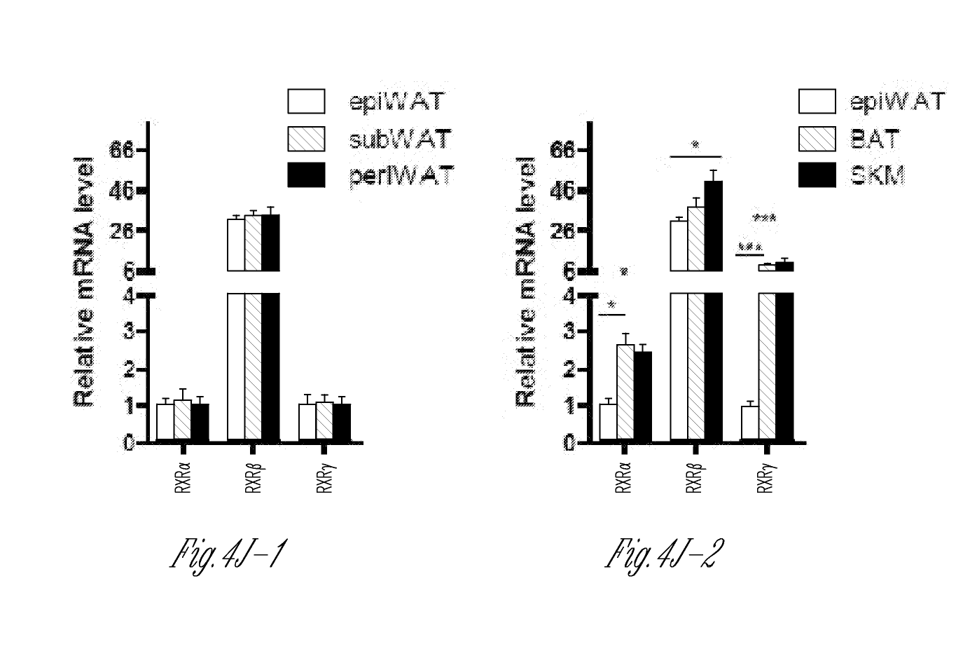

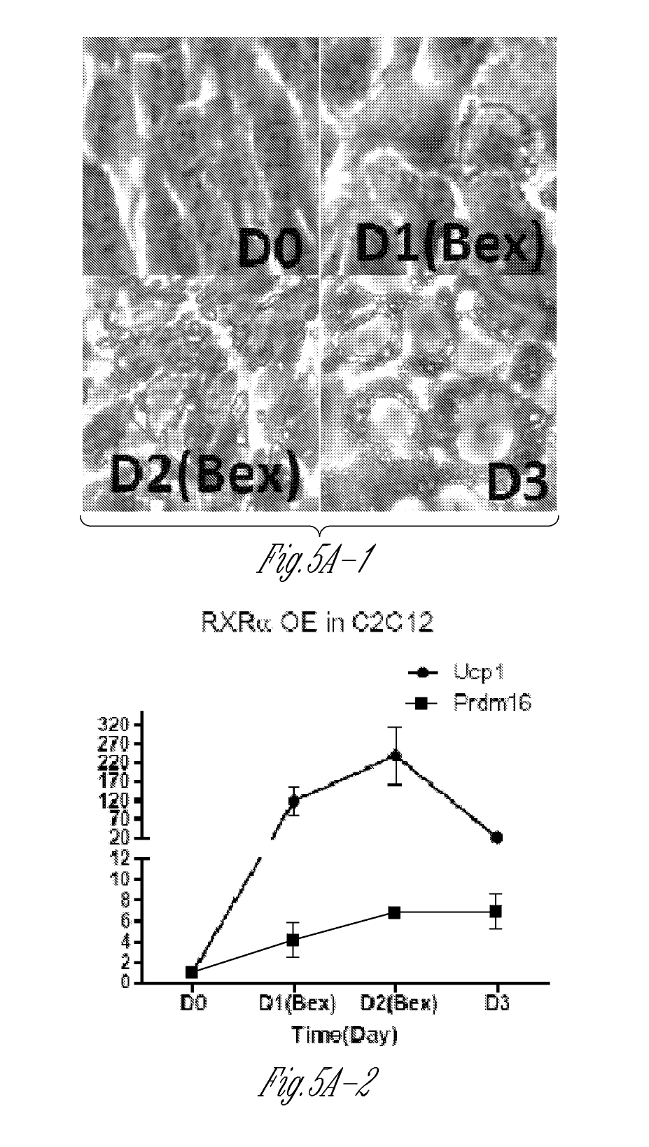

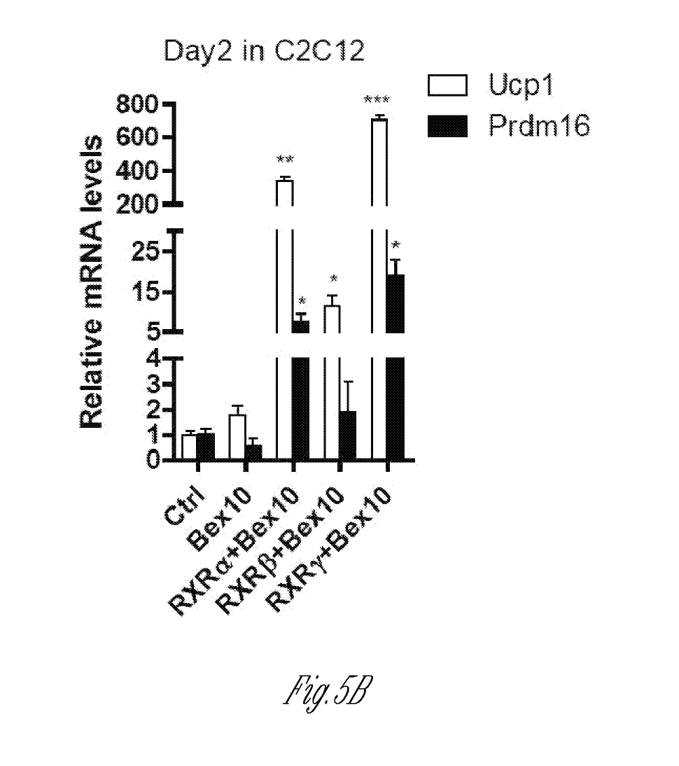

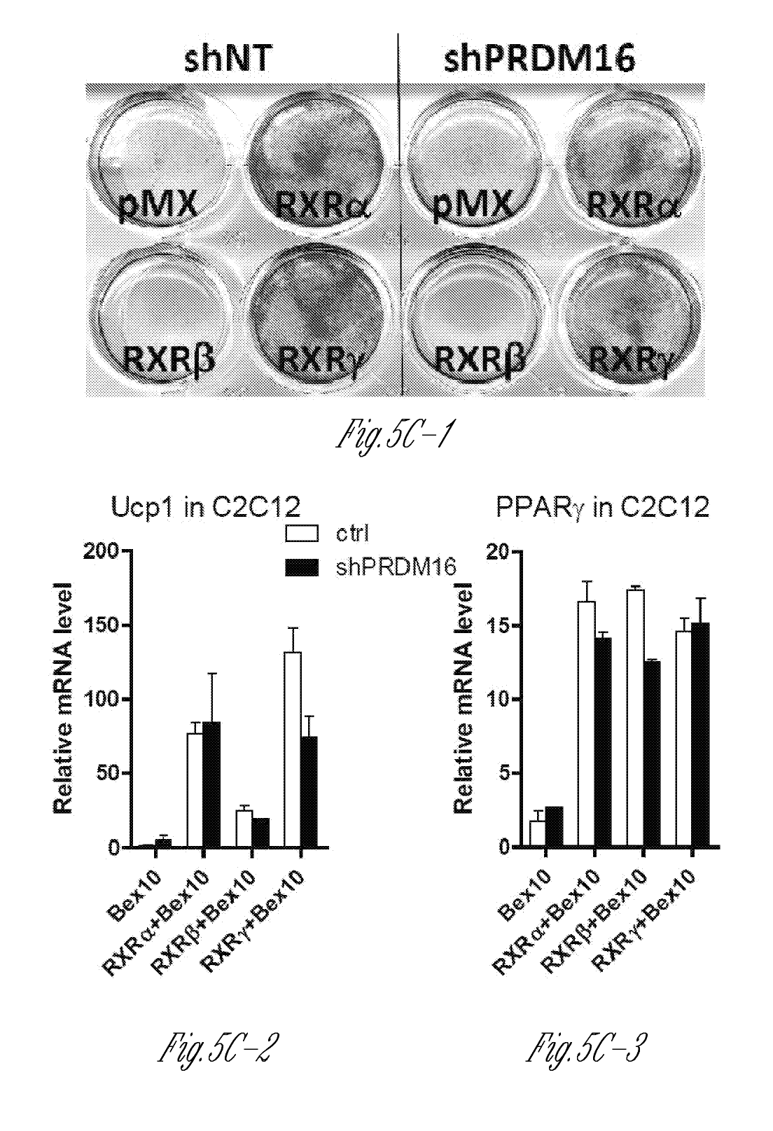

Given bexarotene's specific and remarkable brown adipose tissue-inducing activity in vitro and the existence of relevant cell types in vivo, the inventors next examined whether this reprogramming activity would have any effect in mice. Bexarotene at 50 mg/kg/day was orally administered to normal mice for 4 weeks. Despite similar food intake (FIG. 4A), bexarotene-treated mice had smaller gain in body weight (p=0.0208, FIG. 4B) than control mice. Hence, bexarotene may increase energy expenditure in the mice. To test this possibility, the inventors performed calorimetric analysis of these mice. Bexarotene-treated mice consumed more oxygen (FIG. 4C) and released more CO₂ (FIG. 4D) than the saline-treated group. Furthermore, significantly more total energy was expended, as quantified by heat generation, by the bexarotene-treated group than the saline-treated group (FIG. 4E) without changes in physical activity (data not shown). These data show that bexarotene boosted energy expenditure in vivo.

The masses of various fat depots were also examined at the end of the foregoing experiment. Interscapular brown adipose tissue mass was increased significantly in bexarotene-treated mice than in control mice, both the absolute tissue weight and its portion to body weight (FIG. 4F-1 to 4F-3). However, there was no statistically significant change in white adipose tissue depots (FIGS. 4G-1 and 4G-2), including subcutaneous (subWAT) and epididymal (epiWAT) white adipose tissue. Histological analysis with H&E staining showed similar, if not smaller, lipid droplets in brown adipose tissue (BAT) from bexarotene-treated mice than control mice (FIG. 4H), indicating that the enlarged BAT mass was due to increased brown adipocytes cell number. Moreover, qPCR analysis revealed a marked augmentation of the thermogenic gene expression in brown adipose tissue from bexarotene-treated mice compared control mice, including increased Ucp1 (p=0.0067) and Pgc1α (p=0.0220) (FIG. 4I). Interestingly, Ucp1 expression in subcutaneous white adipose tissue (subWAT) was significantly induced although to a lesser extent (P=0.0065) (FIG. 4I), indicating that bexarotene can also impart the browning effect in subcutaneous white adipose tissue. These data demonstrate that bexarotene exhibits a browning effect in vivo.

### Bexarotene/RXR-Mediated Brown Adipogenesis Partially Depends on PRDM16

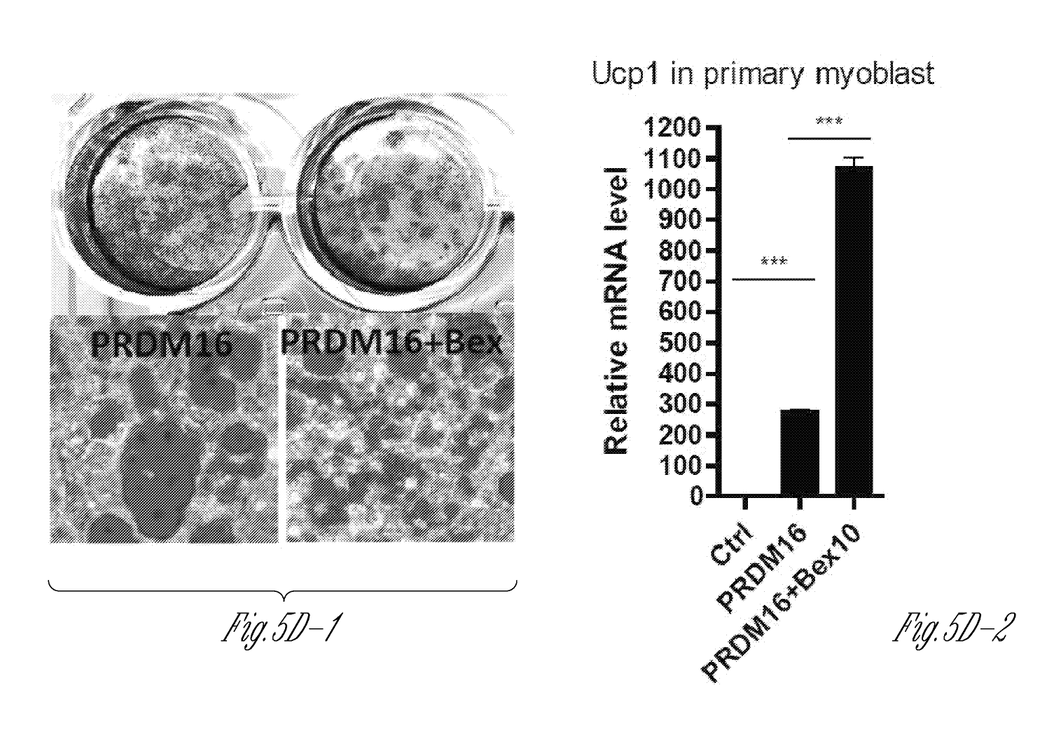

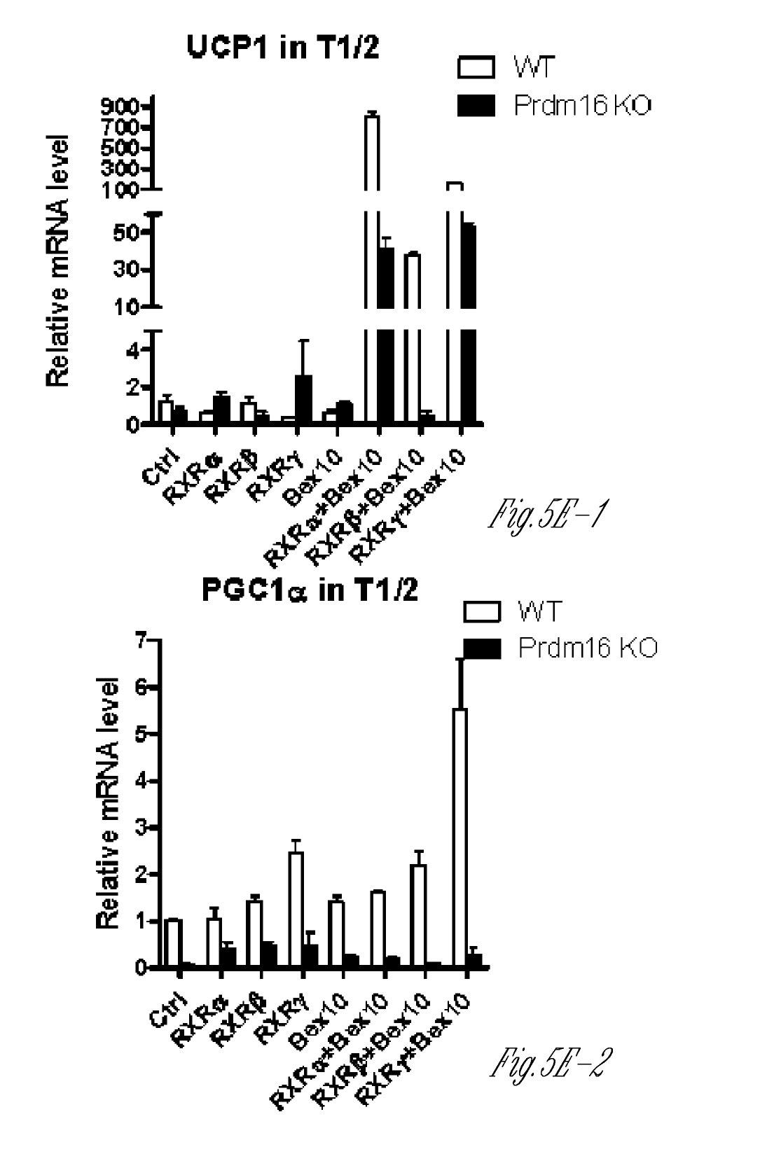

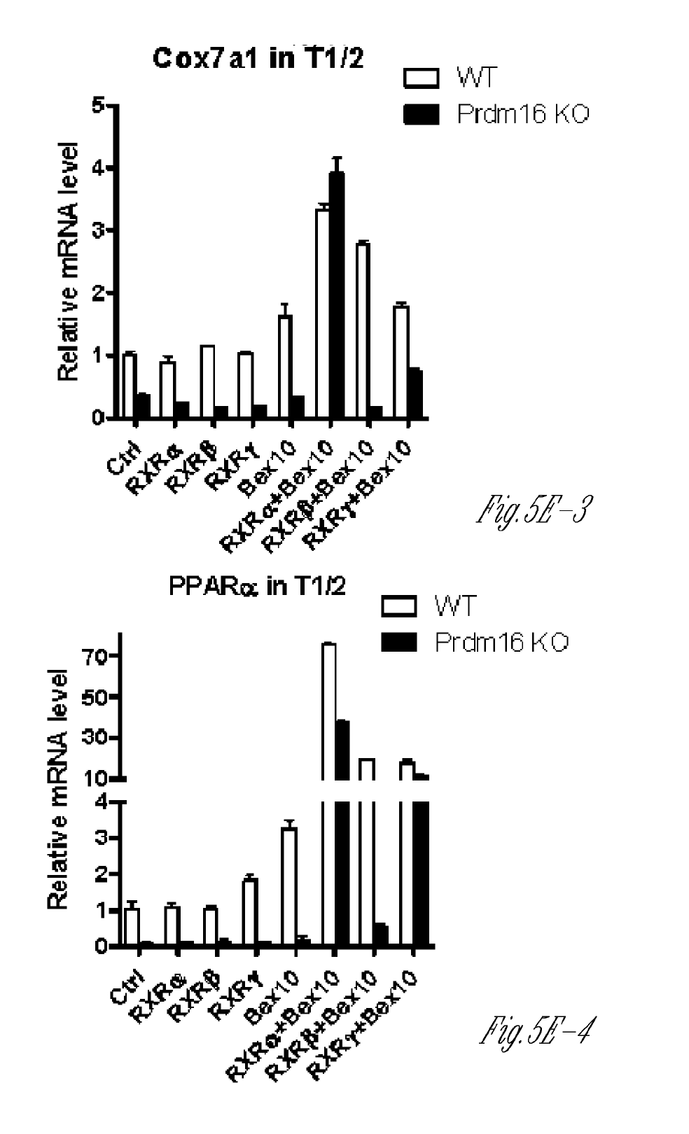

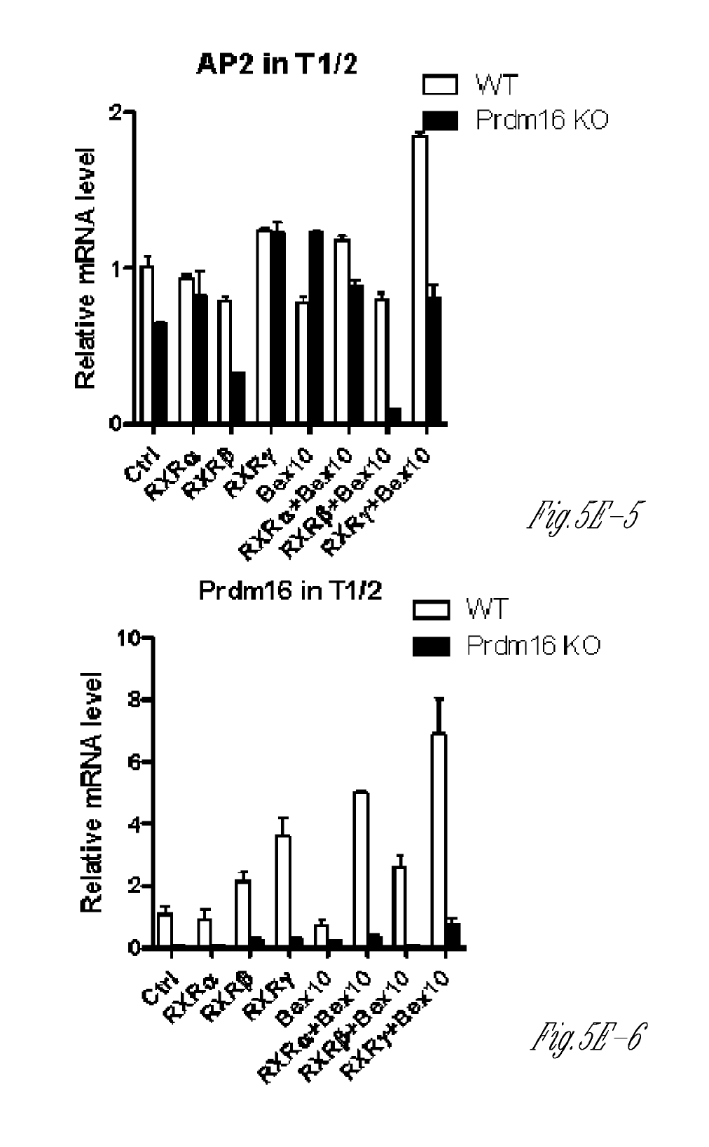

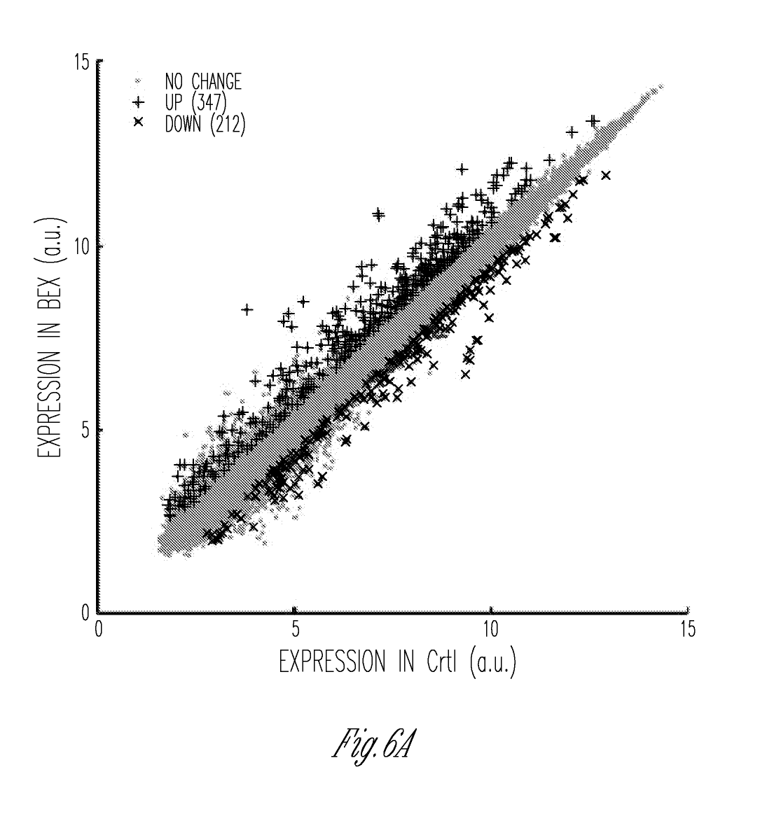

The relationship between retinoid receptors and PRDM16 during brown adipose tissue induction was evaluated by monitoring the gene expression of Prdm16 during the bexarotene/RXR-induced brown adipose tissue reprogramming process. Interestingly, bexarotene induced Prdm16 expression and even more dramatically induced Ucp1 expression in RXRα-overexpressed C2C12 cells at an early stage before typical adipocyte appeared (FIG. 5A). Both RXRα and RXRγ, but not RXRβ, induced Prdm16 expression after bexarotene stimulation for 2 days (FIG. 5B). This result indicated that bexarotene/RXRα/RXRγ may act upstream of PRDM16.

Consistently, knock-down of PRDM16 by shRNA only partially decreased bexarotene/RXR-induced brown adipose tissue reprogramming in C2C12 cells (FIG. 5C-1 to 5C-3) (the Prdm16 knock-down efficiency in pre-BAT is approximately 70%, data not shown). In addition, bexarotene potentiated Prdm16 overexpression-induced Ucp1 expression in primary myoblasts more than fourfold (FIG. 5D-1). Moreover, adding bexarotene to PRDM16 overexpression induced much smaller lipid droplets (i.e., more brown adipose tissue-like) than those in cells with PRDM16 overexpression alone (FIG. 5D-1) indicating that the bexarotene/retinoid X receptor's downstream mechanism is not solely dependent on PRDM16.

In order to further confirm the role of Prdm16 in Bex/RXR-induced brown adipogenesis, CRISPR/Cas9 was employed to knockout Prdm16 gene in C3H10T1/2 cells and to generate a Prdm16 knockout (KO) cell line. As can be seen in FIG. 5E-1 to 5E-6, Prdm16 KO cells possessed similar adipogenesis capacity compared to wild type cells, for example, because AP2 mRNA expression is comparable in most cases (FIG. 5#-5). PGC1α expression is almost completely abolished in Prdm16 KO cells indicating that Prdm16 is essential for mitochondrion biogenesis. As also observed in the Prdm16 knockdown experiments, Bex/RXR-induced Ucp1 expression was partially reduced in Prdm16 KO cells. Other browning genes such as Cox7a1 and PPARα were partially abolished too. These data indicate that Prdm16 is a major downstream component of the Bex/RXR pathway but Bex/RXR activates other downstream signaling to accomplish brown adipogenesis.

### Bexarotene Initiates ‘Browning’ Pathways at an Early Stage of Adipogenesis in C2C12 Cells

PRDM 16 knockdown only partially affected bexarotene/retinoid-induced adipogenesis in C2C12 cells, and neither PRDM16 overexpression in 3T3-L1 nor PRDM16 knockdown in pre-brown adipose tissue affected adipogenesis efficiency (Seale et al., Cell Metab 6: 38-54 (2007)), suggesting that bexarotene/RXR activates downstream regulators other than PRDM16 to mediate the brown adipose tissue reprogramming process.

To further dissect the transcriptional mechanism downstream of bexarotene/RXR, the inventors performed transcriptome analysis of C2C12 cells during early stages of bexarotene treatment corresponding to early adipogenesis (i.e., after 2-days of bexarotene treatment).

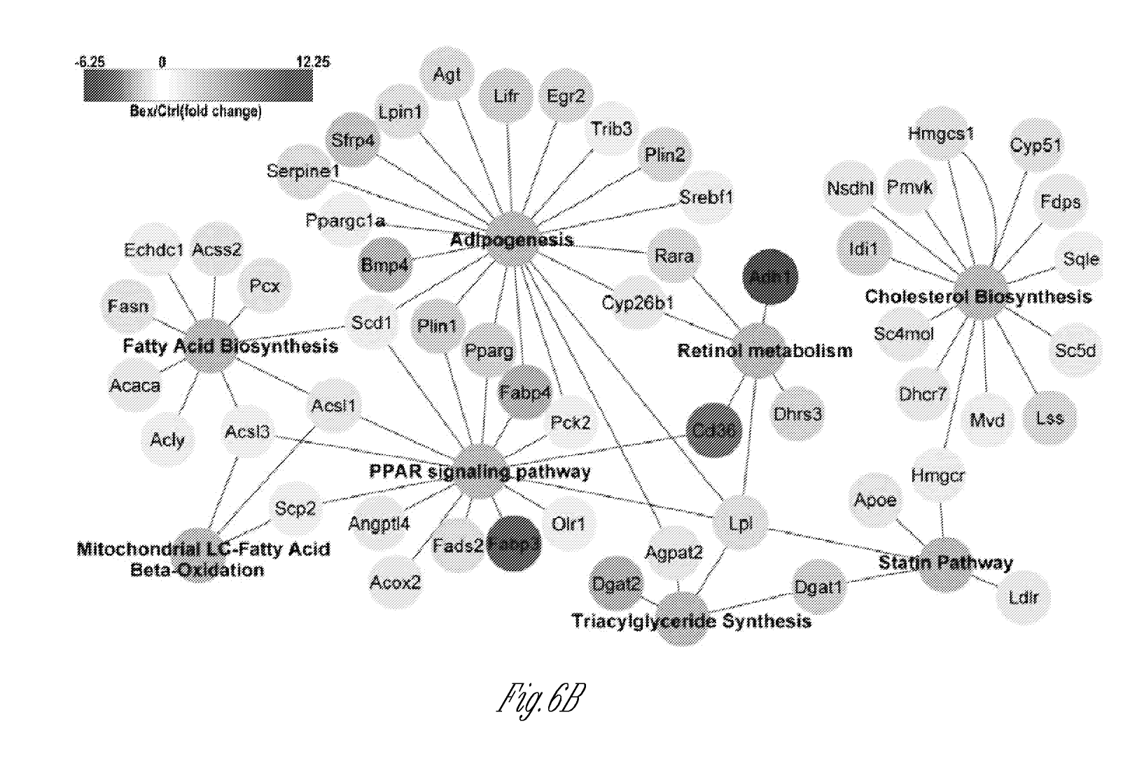

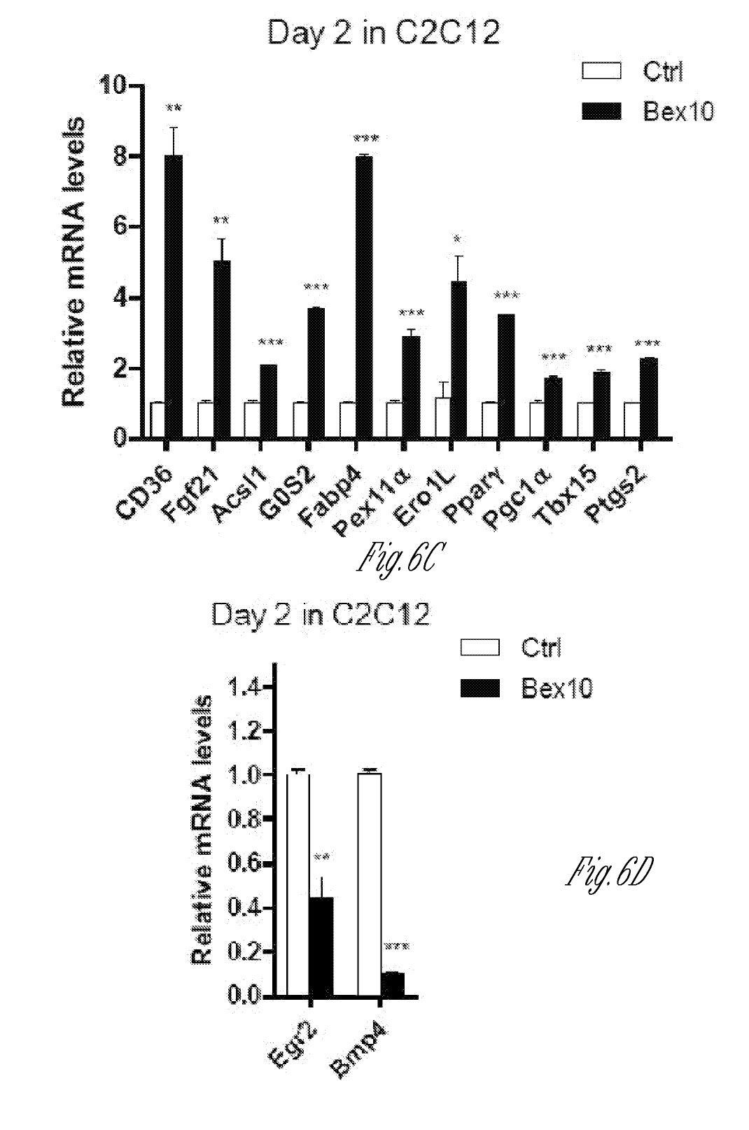

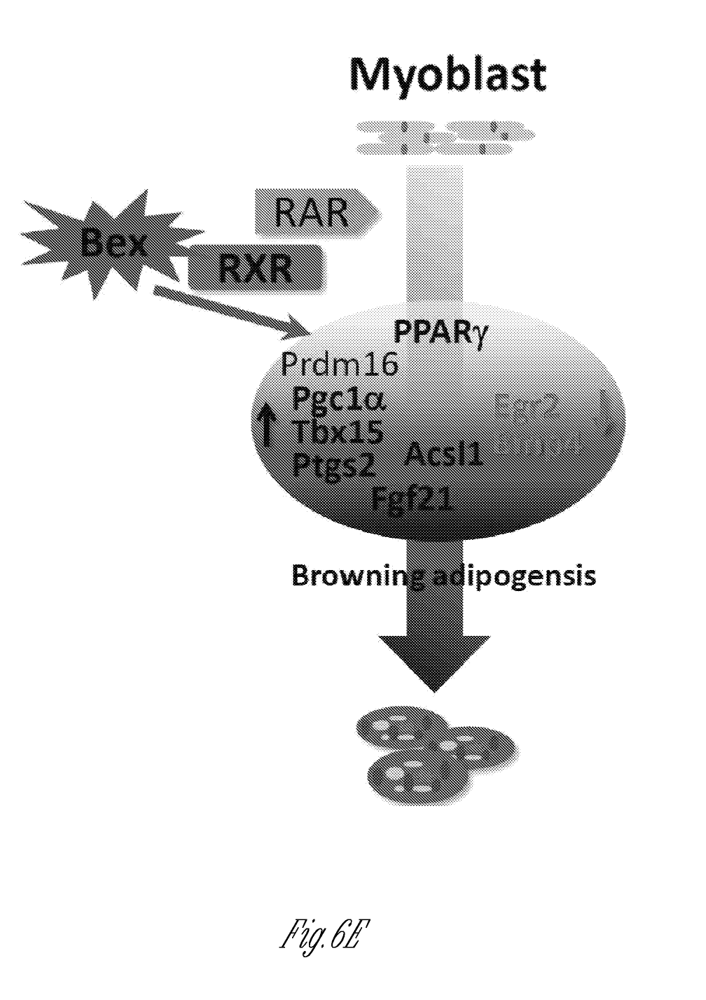

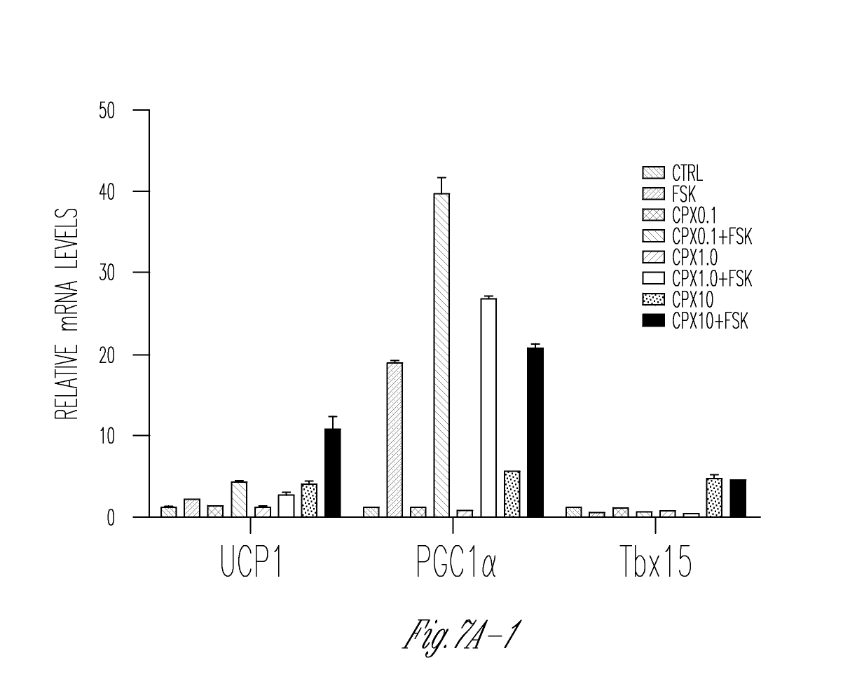

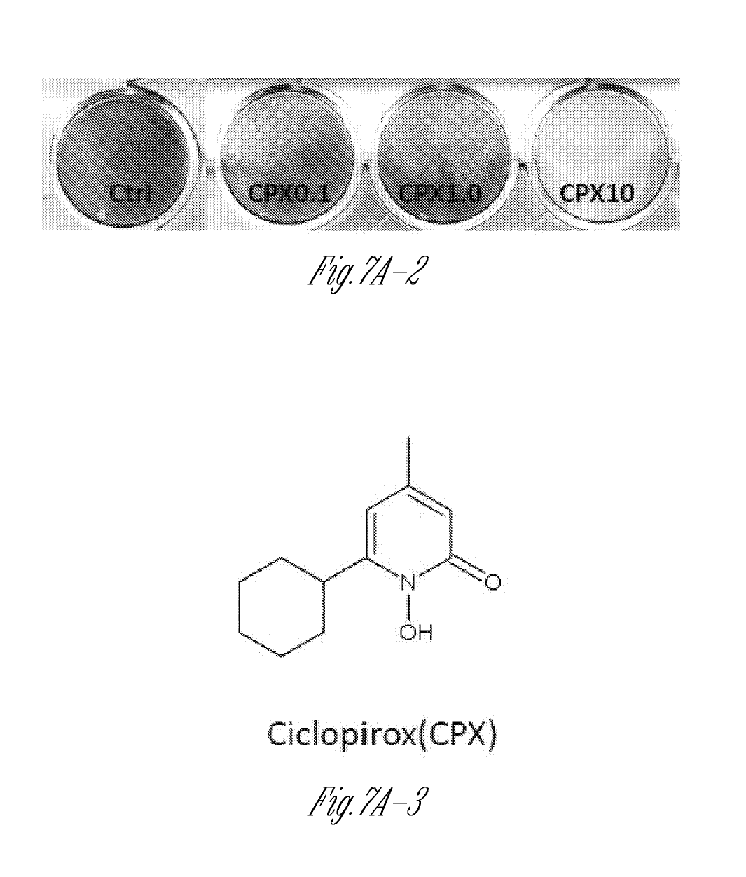

After data normalization, the inventors compared gene expression levels between bexarotene and control (FIG. 6A). DAVID Gene ontology (GO) functional analysis revealed that the genes up-regulated by bexarotene were significantly enriched in those with lipid biogenesis and brown fat cell specification activities (Table 1, cutoff of Benjamini value is <0.01).

TABLE 1
Genes Upregulated by Bexarotene

| GO Term (Biological Process) | Count | % | PValue | Benjamini |
|---|---|---|---|---|
| lipid biosynthetic process | 36 | 10.23 | 4.32E−19 | 7.32E−16 |
| sterol biosynthetic process | 15 | 4.26 | 4.86E−17 | 4.12E−14 |
| cholesterol biosynthetic process | 13 | 3.69 | 1.56E−15 | 8.79E−13 |
| sterol metabolic process | 18 | 5.11 | 4.38E−14 | 1.86E−11 |
| cholesterol metabolic process | 16 | 4.55 | 2.27E−12 | 7.70E−10 |
| steroid biosynthetic process | 16 | 4.55 | 2.83E−12 | 8.00E−10 |
| fatty acid metabolic process | 22 | 6.25 | 3.21E−11 | 7.77E−09 |
| steroid metabolic process | 19 | 5.40 | 1.21E−09 | 2.56E−07 |
| fatty acid biosynthetic process | 13 | 3.69 | 3.17E−08 | 5.97E−06 |
| muscle system process | 12 | 3.41 | 4.00E−08 | 6.78E−06 |
| carboxylic acid biosynthetic | 16 | 4.55 | 5.91E−08 | 9.11E−06 |
| process | | | | |
| organic acid biosynthetic process | 16 | 4.55 | 5.91E−08 | 9.11E−06 |
| muscle contraction | 11 | 3.13 | 1.45E−07 | 2.05E−05 |
| brown fat cell differentiation | 7 | 1.99 | 1.33E−05 | 1.74E−03 |
| lipid homeostatis | 7 | 1.99 | 3.49E−05 | 4.21E−03 |
| circulatory system process | 11 | 3.13 | 4.30E−05 | 4.85E−03 |
| blood circulation | 11 | 3.13 | 4.30E−05 | 4.85E−03 |
| oxidation reduction | 29 | 8.24 | 5.96E−05 | 6.30E−03 |
| coenzyme metabolic process | 12 | 3.41 | 7.70E−05 | 7.66E−03 |

Interestingly, there was no significant GO function enrichment in down-regulated genes by bexarotene (data not shown). To identify the minimal non-redundant set of pathways involving in this process, these genes were further analyzed by GO-Elite and visualized by Cytoscape (Shannon et al., Genome Res 13, 2498-2504 (2003)). Most pathways induced by bexarotene were implicated in adipocyte development, including adipogenesis, PPAR pathway, triacylglyceride synthesis, statin pathway, retinol metabolism, fatty acid biogenesis pathways, cholesterol biosynthesis and mitochondrial LC-fatty acid beta-oxidation (FIG. 6B).

By close inspection and experimental confirmation with qPCR, bexarotene significantly increased levels of the general adipogenesis markers PPARγ, Fabp4 (also known as aP2), and CD36, as well as some downstream targets of RXR/PPARγ (e.g., Pex11a, Angpt14, and HK2 (Nielsen et al., Genes Dev 22, 2953-2967 (2008)).

In addition, bexarotene increased expression of some genes with known ‘browning’ effect, such as Fgf21, Pgc1α, Ppargc1a, Acsl1, Tbx15, Ptgs2 and G0S2 (Table 2 and FIG. 6C-6D).

TABLE 2
Expression level fold-changes
of some adipogenesis and browning genes

| Gene | Bex/Ctrl (fold) |
|---|---|
| Cd36 | 9.08 |
| Fabp4 | 5.43 |
| Pparg | 3.51 |
| Pex11a | 3.08 |
| Angptl4 | 1.91 |
| Hk2 | 1.8 |
| G0s2 | 3.09 |
| Fgf21 | 2.88 |
| Ptgs2 | 2.61 |
| Acsl1 | 2.17 |
| Ppargc1a | 1.65 |
| Tbx15 | 1.61 |
| Egr2 | −1.56 |
| Bmp4 | −2.46 |

Fgf21 has a physiological role in thermogenesis of WATs. FG F21^{-/-} mice displayed impaired ability to adapt to chronic cold exposure, with diminished browning of WAT (Fisher et al., Genes Dev 26, 271-281 (2012)).

Pgc1α is the key regulator of mitochondrial biogenesis and function (Ventura-Clapier et al., Cardiovasc Res 79, 208-217 (2008)) and is essential to brown adipose tissue function (Lin et al., Cell 119, 121-135 (2004)).

Acsl1 is responsible for mitochondrial long chain-fatty acid β-oxidation and required for cold thermogenesis (Ellis et al., Cell Metab 12, 53-64 (2010).

Tbx15 is essential for brown and brite adipocyte but not for white adipocyte differentiation (Gburcik et al., Am J Physiol Endocrinol Metab 303, E1053-1060 (2012)). Moreover, 3T3-L1 white adipose tissue differentiation was impaired by Tbx15 overexpression (Gesta et al., Proc Natl Acad Sci USA 108: 2771-2776 (2011)).

Overexpression of Ptgs2 (also known as COX-2) in white adipose tissue induced de novo browning recruitment in white adipose tissue, increased systemic energy expenditure, and protected mice against high-fat diet-induced obesity (Madsen et al., PLoS One 5, e11391 (2010); Vegiopoulos et al., Science 328, 1158-1161 (2010)).

G0S2 (the G0/G1 switch gene 2) expression is also induced along adipogenesis, and its expression negatively correlates with the development of obesity (Yang et al., Cell Metab 11: 194-205 (2010)).

On the other hand, bexarotene repressed some white adipogenesis-associated genes, such as Bmp4 and Egr2 (also known as KROX20) (Table 2). Bmp4 specifically promotes white adipocyte differentiation of C3H10T1/2 cells and even lower UCP1 level in pre-BAT (Tseng et al., Nature 454, 1000-1004 (2008). The transcription factor Egr2 was reported to be essential for 3T3-L1 cellular differentiation via the transcriptional activator of C/EBPB. Conversely, knockdown of Egr2 reduced adipogenesis in 3T3-L 1 cells (Chen et al., Cell Metab 1: 93-106 (2005)).

These data provide a molecular pathway map for browning induction in C2C 12 cells, and show that bexarotene/RXR is a master regulator of brown adipose tissue specification. FIG. 6E illustrates early stage bexarotene RXR-mediated browning adipogenesis pathways. The data provided herein also show that bexarotene/RXR activates a spectrum of general adipogenesis pathways, particularly those that specifically induce brown adipose tissue, while suppressing white adipose tissue-selective genes.

### EXAMPLE 3

### Ciclopirox and IOX2 Induce Brown Adipose Tissue Markers

This Example illustrates the effects of ciclopirox and IOX2 upon expression of genes associated with the brown adipose tissue phenotype.

C3H10T1/2 cells were incubated in concentrations of ciclopirox and IOX2 that varied from 0.1 μM to 50 μM. Gene expression levels of genes correlated with the brown adipose tissue phenotype was measured using procedures described in Example 1.

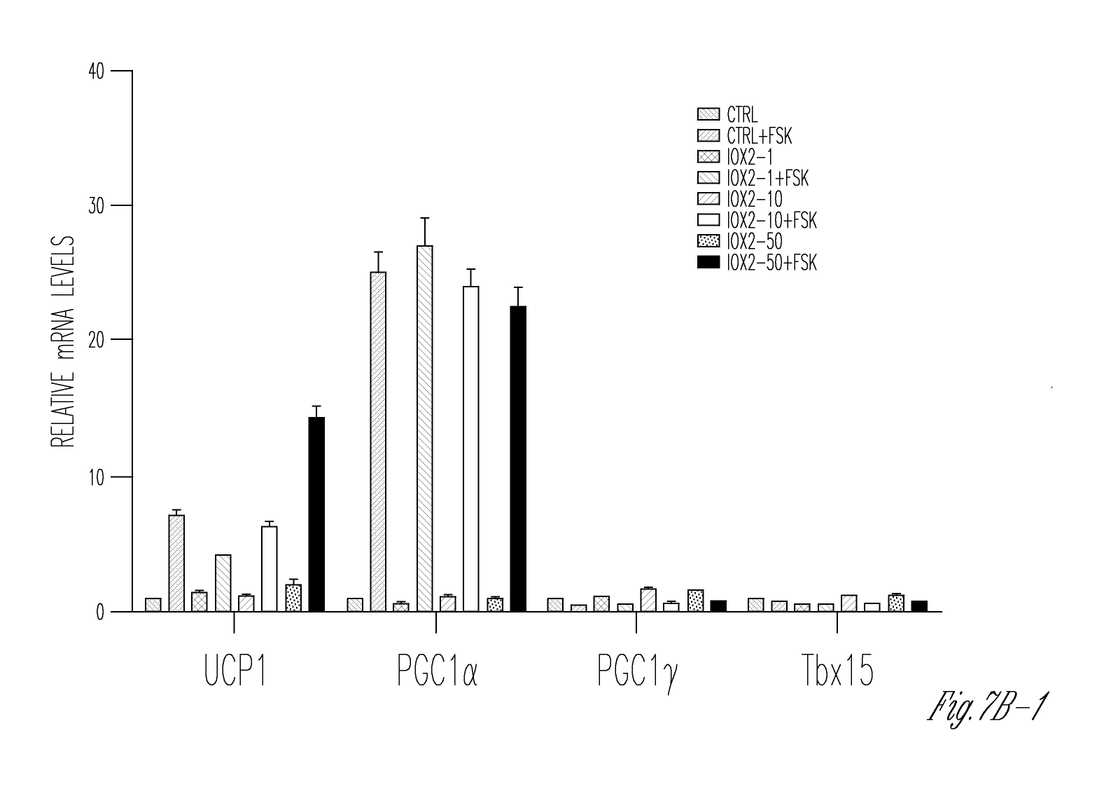

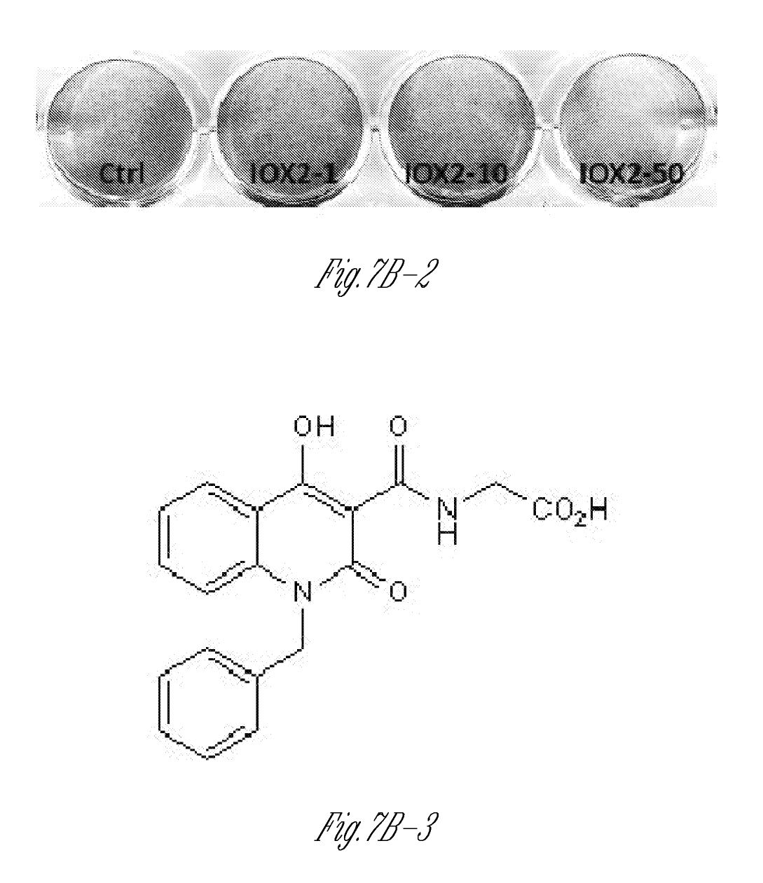

Gene expression analysis showed that ciclopirox and IOX2 significantly induced the expression of adipogenesis maker PPARγ, and expression of several brown adipocyte-specific genes (FIG. 7A-7B). Furthermore, these BAT-like adipocytes readily responded to forskolin (Fsk) to induce thermogenic genes Ucp1 and Pgc1α (FIG. 7A-7B).

### EXAMPLE 4

### Bexarotene Treatment can Reduce Body Weight

This Example illustrates that bexarotene treatment can reduce body weight gain in mammals.

### Methods

Male mice (8-week-old, C57BL/6J) were housed in metabolism cages and maintained on a 12 h light-dark cycle at 23° C. The mice were fed a high fat diet (HFD) (21.9 kJ/g, 60% of energy as fat, 20% of energy as protein, 20% of energy as carbohydrate; D12492; Research Diet, New Brunswick, N.J., USA) for four weeks. The mice received a daily intraperitoneal injection with saline or bexarotene (50 mg/kg) until the study was ended.

Glucose and insulin tolerance tests were performed on the mice at the end of the four week dosing period. Mice were fasted overnight and injected intraperitoneally (i.p.) with 20% glucose at a dose of 2 g/kg body weight for glucose tolerance tests (GTTs). For insulin tolerance tests (ITTs), mice were starved for 6 h and i.p. injected with 0.5 U/kg body weight of recombinant human insulin (Eli Lilly). Blood glucose was monitored from the tail vein blood using a glucometer (ACCU-CHEK Advantage; Roche Diagnostics China, Shanghai, China) at various time points. Mouse body weight, subcutaneous (inguinal fat; SubQ fat), and epididymal adipose tissue weight (epidi weight) were also determined at the end of the 4 week test.

### Results

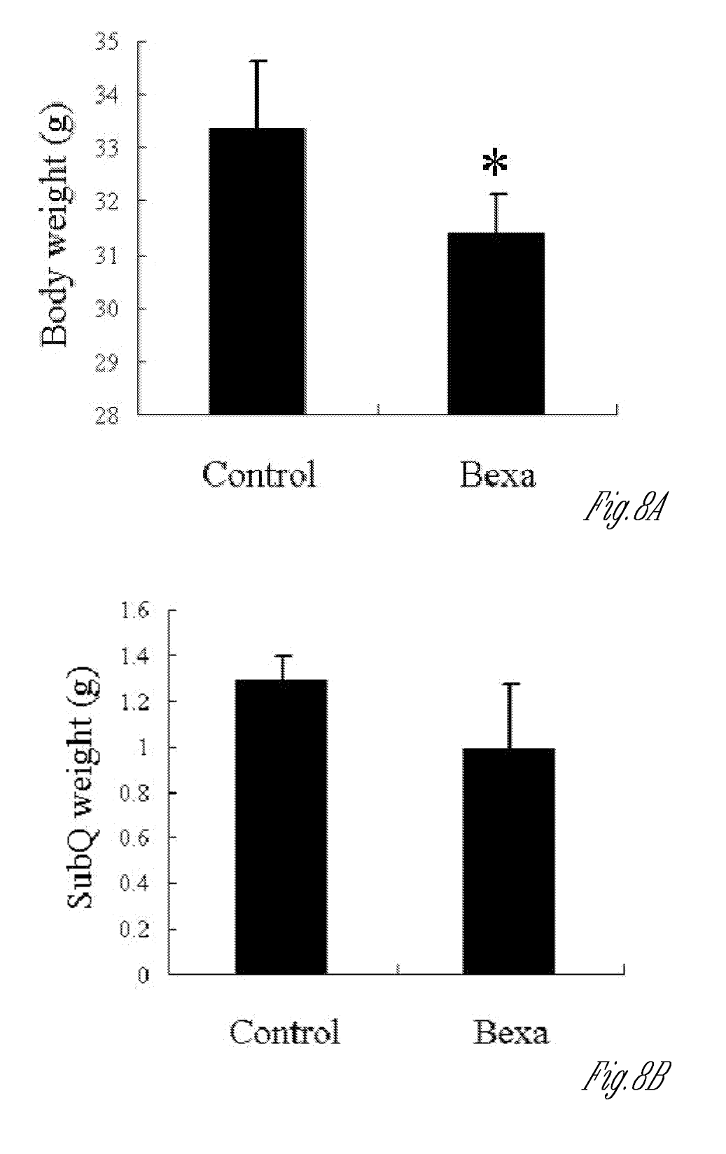

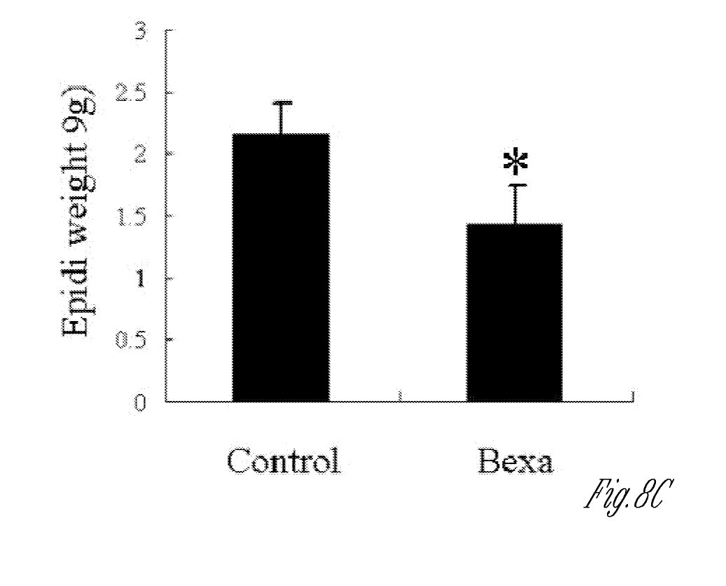

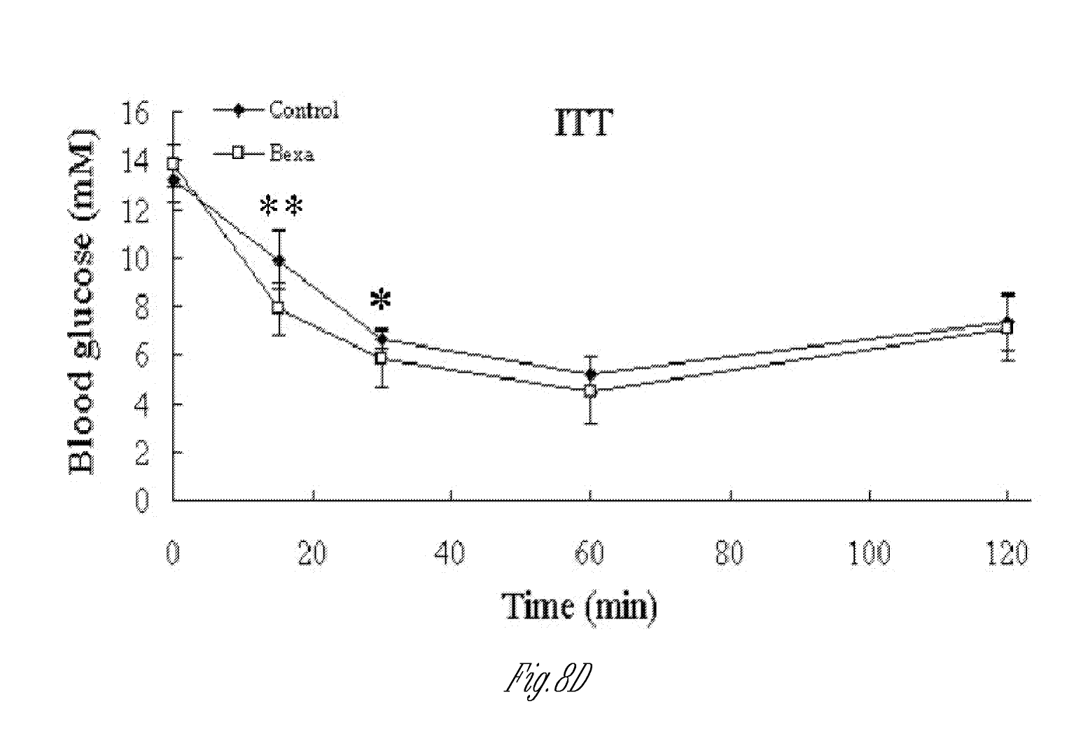

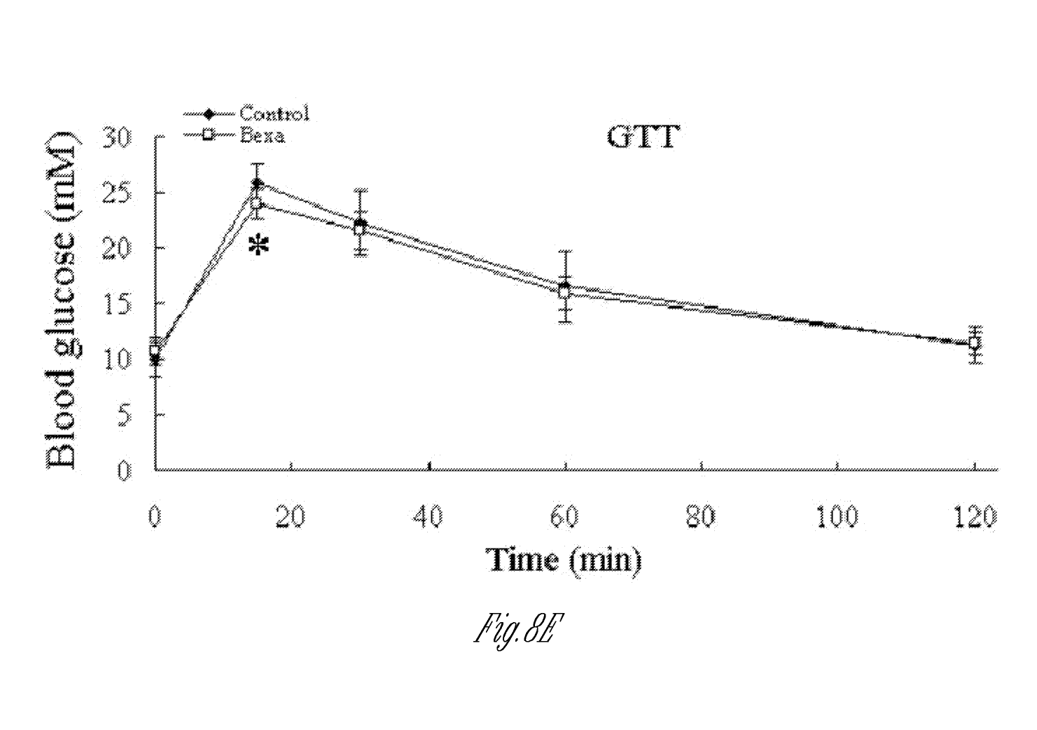

As shown in FIG. 8A-8C, bexarotene-treated mice had lower body weight (FIG. 8A), less subcutaneous fat (FIG. 8B), and less epididymal adipose tissue (FIG. 8C) than control mice that did not receive bexarotene. Insulin tolerance tests showed that bexarotene-treated mice responded to insulin more sensitively (FIG. 8D). Glucose tolerance tests showed that bexarotene-treated mice have improved glucose homeostasis upon glucose challenge (FIG. 8E).

### REFERENCES

Bartelt, A., Bruns, O. T., Reimer, R., Hohenberg, H., lttrich, H., Peldschus, K., Kaul, M. G., Tromsdorf, U. l., Weller, H., Waurisch, C., et al. (2011). Brown adipose tissue activity controls triglyceride clearance. Nat Med 17, 200-205.
Cannon, B., and Nedergaard, J. (2004). Brown adipose tissue: function and physiological significance. Physiol Rev 84, 277-359.
Chen, Z., Torrens, J. l., Anand, A., Spiegelman, B. M., and Friedman, J. M. (2005). Krox20 stimulates adipogenesis via C/EBPbeta-dependent and -independent mechanisms. Cell Metab 1, 93-106.
Cypess, A. M., Lehman, S., Williams, G., Tal, l., Rodman, D., Goldfine, A. B., Kuo, F. C., Palmer, E. L., Tseng, Y. H., Doria, A., et al. (2009). Identification and importance of brown adipose tissue in adult humans. N Engl J Med 360, 1509-1517.
Cypess, A. M., White, A. P., Vernochet, C., Schulz, T. J., Xue, R., Sass, C A, Huang, T. L, Roberts-Toler, C., Weiner, L. S., Sze, C., et al. (2013). Anatomical localization, gene expression profiling and functional characterization of adult human neck brown fat. Nat Med 19, 635-639.
Ellis, J. M., Li, L. O., Wu, P. C., Koves, T. R., llkayeva, 0., Stevens, R. D., Watkins, S. M., Muoio, D. M., and Coleman, R. A. (2010). Adipose acyl-CoA synthetase-1 directs fatty acids toward beta-oxidation and is required for cold thermogenesis. Cell Metab 12, 53-64.
Enderlin, V., Pallet, V., Alfos, S., Dargelos, E., Jaffard, R., Garcin, H., and Higueret, P. (1997). Age-related decreases in mRNA for brain nuclear receptors and target genes are reversed by retinoic acid treatment. Neurosci Lett 229, 125-129.
Feldmann, H. M., Golozoubova, V., Cannon, B., and Nedergaard, J. (2009). UCP1 ablation induces obesity and abolishes diet-induced thermogenesis in mice exempt from thermal stress by living at thermoneutrality. Cell Metab 9, 203-209.
Fisher, F. M., Kleiner, S., Douris, N., Fox, E. C., Mepani, R. J., Verdeguer, F., Wu, J., Kharitonenkov, A., Flier, J. S., Iviaratos-Fiier, E., et al. (2012). FGF21 regulates PGC-1alpha and browning of white adipose tissues in adaptive thermogenesis. Genes Dev 26, 271-281.
Gburcik, V., Cawthorn, W. P., Nedergaard, J., Timmons, J. A., and Cannon, B. (2012). An essential role for Tbx15 in the differentiation of brown and 11 brite11 but not white adipocytes. Am J Physiol Endocrinol Metab 303, E1053-1060.
Gesta, S., Bezy, 0., Mori, M. A., Macotela, Y., Lee, K. Y., and Kahn, C. R. (2011). Mesodermal developmental gene Tbx15 impairs adipocyte differentiation and mitochondrial respiration. Proc Natl Acad Sci USA 108, 2771-2776.
Jayawardena, T. M., Egemnazarov, B., Finch, E. A., Zhang, L., Payne, J. A., Pandya, K., Zhang, Z., Rosenberg, P., Mirotsou, M., and Dzau, V. J. (2012). MicroRNA-mediated in vitro and in vivo direct reprogramming of cardiac fibroblasts to cardiomyocytes. Circ Res 110, 1465-1473.
Udell, M. E., Betz, M. J., Dahlqvist Leinhard, O., Heglind, M., Elander, L., Slawik, M., Mussack, T., Nilsson, D., Romu, T., Nuutila, P., et al. (2013). Evidence for two types of brown adipose tissue in humans. Nat Med 19, 631-634.
Lin, l, Wu, P. H., Tarr, P. T., Lindenberg, K. S., St-Pierre, J., Zhang, C. Y., Mootha, V. K., Jager, S., Vianna, C. R., Reznick, R. M., et al. (2004). Defects in adaptive energy metabolism with CNS-linked hyperactivity in PGC-1alpha null mice. Cell 119, 121-135.
Liu, X., Zheng, Z., Zhu, X., Meng, M., Li, L., Shen, Y., Chi, Q., Wang, D., Zhang, Z., Li, C., et al. (2013). Brown adipose tissue transplantation improves whole-body energy metabolism. Cell Res 23, 851-854.
Madsen, L., Pedersen, L. M., Lillefosse, H. H., Fjaere, E., Bronstad, l., Hao, Q., Petersen, R. K., Hallenborg, P., Ma, T., DeMatteis, R., et al. (2010). UCP1 induction during recruitment of brown adipocytes in white adipose tissue is dependent on cyclooxygenase activity. PLoS One 5, e11391.
Mecocci, P., Polidori, M. C., Troiano, L., Cherubini, A., Cecchetti, R., Pini, G., Straatman, M., Monti, D., Stahl, W., Sies, H., et al. (2000). Plasma antioxidants and longevity: a study on healthy centenarians. Free Radio Biol Med 28, 1243-1248.
Mingaud, F., Mormede, C., Etchamendy, N., Mons, N., Niedergang, B., Wietrzych, M., Pallet, V., Jaffard, R., Krezel, W., Higueret, P., et al. (2008). Retinoid hyposignaling contributes to aging-related decline in hippocampal function in short-term/working memory organization and long-term declarative memory encoding in mice. J Neurosci 28, 279-291.
Nielsen, R., Pedersen, T. A., Hagenbeek, D., Moulos, P., Siersbaek, R., Megens, E., Denissov, S., Borgesen, M., Francoijs, K. J., Mandrup, S., et al. (2008). Genome-wide profiling of PPARgamma:RXR and RNA polymerase II occupancy reveals temporal activation of distinct metabolic pathways and changes in RXR dimer composition during adipogenesis. Genes Dev 22, 2953-2967.
Ohno, H., Shinoda, K., Spiegelman, B. M., and Kajimura, S. PPARgamma agonists Induce a White-to-Brown Fat Conversion through Stabilization of PRDM16 Protein. Cell Metab 15, 395-404.
Ohno, H., Shinoda, K., Spiegelman, B. M., and Kajimura, S. (2012). PPARgamma agonists induce a white-to-brown fat conversion through stabilization of PRDM16 protein. Cell Metab 15, 395-404.
Ouellet, V., Routhier-Labadie, A., Bellemare, W., Lakhai-Chaieb, L., Turcotte, E., Carpentier, A. C., and Richard, D. (2011). Outdoor temperature, age, sex, body mass index, and diabetic status determine the prevalence, mass, and glucose-uptake activity of 18F-FDG-detected BAT in humans. J Clin Endocrine Metab 96, 192-199.
Pallet, V., Azais-Braesco, V., Enderlin, V., Grolier, P., Noei-Suberville, C., Garcin, H., and Higueret, P. (1997). Aging decreases retinoic acid and triiodothyronine nuclear expression in rat liver: exogenous retinol and retinoic acid differentially modulate this decreased expression. Mech Ageing Dev 99, 123-136.
Pfannenberg, C., Werner, M. K., Ripkens, S., Stef, l., Deckert, A., Schmadl, M., Reimold, M., Haring, H. U., Claussen, C. D., and Stefan, N. (2010). Impact of age on the relationships of brown adipose tissue with sex and adiposity in humans. Diabetes 59, 1789-1793.
Qian, L., Huang, Y., Spencer, C. L., Foley, A., Vedantham, V., Liu, L., Conway, S. J., Fu, J. D., and Srivastava, D. (2012). In vivo reprogramming of murine cardiac fibroblasts into induced cardiomyocytes. Nature 485, 593-598.
Rajakumari, S., Wu, J., Ishibashi, J., Lim, H. W., Giang, A. H., Won, K. J., Reed, R. R., and Seale, P. (2013). EBF2 determines and maintains brown adipocyte identity. Cell Metab 17, 562-574.
Seale, P., Bjork, B., Yang, W., Kajimura, S., Chin, S., Kuang, S., Scime, A., Devarakonda, S., Conroe, H. M., Erdjument-Bromage, H., et al. (2008). PRDM16 controls a brown fat/skeletal muscle switch. Nature 454, 961-967.
Seale, P., Conroe, H. M., Estall, J., Kajimura, S., Frontini, A., Ishibashi, J., Cohen, P., Cinti, S., and Spiegelman, B. M. (2011). Prdm16 determines the thermogenic program of subcutaneous white adipose tissue in mice. J Clin Invest 121, 96-105.
Seale, P., Kajimura, S., Yang, W., Chin, S., Rohas, L. M., Uldry, M., Tavernier, G., Langin, D., and Spiegelman, B. M. (2007). Transcriptional control of brown fat determination by PRDM16. Cell Metab 6, 38-54.
Shannon, P., Markiel, A., Ozier, 0., Baliga, N. S., Wang, J. T., Ramage, D., Amin, N., Schwikowski, B., and Ideker, T. (2003). Cytoscape: a software environment for integrated models of biomolecular interaction networks. Genome Res 13, 2498-2504.
Siersbaek, M. S., Loft, A., Aagaard, M. M., Nielsen, R., Schmidt, S. F., Petrovic, N., Nedergaard, J., and Mandrup, S. (2012). Genome-wide profiling of peroxisome proliferator-activated receptor gamma in primary epididymal, inguinal, and brown adipocytes reveals depot-selective binding correlated with gene expression. Mol Cell Biol 32, 3452-3463.
Stanford, K. I., Middelbeek, R. J., Townsend, K. L., An, D., Nygaard, E. B., Hitchcox, K. M., Markan, K. R., Nakano, K., Hirshman, M. F., Tseng, Y. H., et al. (2013). Brown adipose tissue regulates glucose homeostasis and insulin sensitivity. J Clin Invest 123, 215-223.
Torper, O., Pfisterer, U., Wolf, D. A., Pereira, M., Lau, S., Jakobsson, J., Bjorklund, A., Grealish, S., and Parmar, M. (2013). Generation of induced neurons via direct conversion in vivo. Proc Natl Acad Sci USA 110, 7038-7043.
Tseng, Y. H., Kokkotou, E., Schulz, T. J., Huang, T. L., Win nay, J. N., Taniguchi, C. M., Tran, T. T., Suzuki, R., Espinoza, D. O., Yamamoto, Y., et al. (2008). New role of bone morphogenetic protein 7 in brown adipogenesis and energy expenditure. Nature 454, 1000-1004.
van Marken Lichtenbelt, W. O., Vanhommerig, J. W., Smulders, N. M., Drossaerts, J. M., Kemerink, G. J., Bouvy, N. D., Schrauwen, P., and Teule, G. J. (2009). Cold-activated brown adipose tissue in healthy men. N Engl J Med 360, 1500-1508.
Vegiopoulos, A., Muller-Decker, K., Strzoda, D., Schmitt, l., Chichelnitskiy, E., Ostertag, A., Berriel, Diaz, M., Rozman, J., Hrabe de Angelis, M., Nusing, R. M., et al. (2010). Cyclooxygenase-2 controls energy homeostasis in mice by de novo recruitment of brown adipocytes. Science 328, 1158-1161.
Ventura-Ciapier, R., Gamier, A., and Veksler, V. (2008). Transcriptional control of mitochondrial biogenesis: the central role of PGC-1alpha. Cardiovasc Res 79, 208-217.
Virtanen, K. A., Udell, M. E., Oraval J., Heglind, M., Westergren, R., Niemi, T., Taittonen, M., Laine, J., Savisto, N. J., Enerback, S., et al. (2009). Functional brown adipose tissue in healthy adults. N Engl J Med 360, 1518-1525.
Wu, J., Bostrom, P., Sparks, L. M., Ye, L., Choi, J. H., Giang, A. H., Khandekar, M., Virtanen, K. A., Nuutila, P., Schaart, G., et al. (2012). Beige adipocytes are a distinct type of thermogenic fat cell in mouse and human. Cell 150, 366-376.
Xiao, F., Wang, H., Fu, X., Li, Y., Ma, K., Sun, L., Gao, X., and Wu, Z. (2011). Oncostatin M inhibits myoblast differentiation and regulates muscle regeneration. Cell Res 21, 350-364.
Yang, X., Lu, X., Lombes, M., Rha, G. B., Chi, V. I., Guerin, T. M., Smart, E. J., and Liu, J. (2010). The G(O)/G(1) switch gene 2 regulates adipose lipolysis through association with adipose triglyceride lipase. Cell Metab 11, 194-205.
Zambon, A. C., Gaj, S., Ho, l., Hanspers, K., Vranizan, K., Evelo, C. T., Conklin, B. R., Pico, A. R., and Salomonis, N. (2012). GO-Elite: a flexible solution for pathway and ontology over-representation. Bioinformatics 28,2209-2210.
Zhou, Q., Brown, J., Kanarek, A., Rajagopal, J., and Melton, D. A. (2008). In vivo reprogramming of adult pancreatic exocrine cells to beta-cells. Nature 455, 627-632.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification

### Statements:

1. A method of generating one or more brown adipose cells from one or more starting cells, comprising contacting the one or more starting cells with bexarotene, ciclopirox, IOX2, or combinations thereof, to thereby generate one or more brown adipose cells.
2. The method of statement 1, wherein the starting cells are selected from the group of myoblasts, adipocytes, pre-adipocytes, mesenchymal precursor cells, multipotent stem cells, pluripotent stem cells, unipotent stem cells, fibroblasts, white adipocytes, and any combination thereof.
3. The method of statement 1 or 2, which inhibits white adipocyte cell generation.
4. The method of any of statements 1-3, further comprising contacting the one or more starting cells with retinoic acid, 9-cis retinoic acid, all-trans 3,4-didehydro retinoic acid, 4-oxo retinoic acid, retinol, rosigliotazone, forskolin, or any combination thereof.
5. The method of any of statements 1-4, further comprising contacting the one or more starting cells with one or more retinoids of formula I:

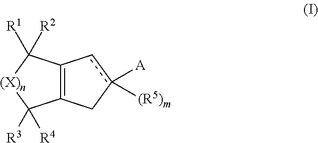

wherein:
   the dotted bond is either present and forms a double bond, or is absent;
   R¹, R², R³ and R⁴ are independently hydrogen or alkyl;
   n is 1, 2 or 3;
   X is —C(R⁸)(R⁹)— for n=1, 2 or 3; or X is oxygen for n=1;
   R⁸ and R⁹ are independently hydrogen or alkyl;
   R is hydrogen, alkyl, alkoxy, alkoxy-alkyl-, alkylthio, alkyl-NR¹⁰—, alkenyl, alkenyloxy, alkynyl, benzyl, cycloalkyl-alkyl, phenyl-alkyl, R¹⁰ is hydrogen or alkyl;
   m is 0 when the dotted bond is present; and m is 1 when the dotted bond is absent; and
   A is a residue of formula:

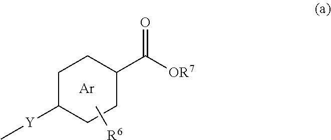

or of formula:

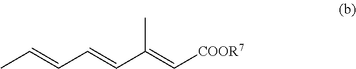

wherein
   Ar is phenyl or a heteroarylic ring;
   R⁶ is hydrogen, halogen, alkoxy or hydroxy;
   R⁷ is hydrogen or alkyl; and Y is —COO—, —OCO—, —CONR¹⁰—, —NR¹⁰CO—, —CH═CH—, —C≡C—, —COCH═CH—, —CHOHCH═CH—, —CH₂0-, —CH₂S—, —CH₂SO—, —CH₂S0₂-, —CH₂NR¹⁰—, —OCH₂—, —SCH₂—, —SOCH₂—, —S0₂CH₂— or —NR¹⁰CH₂—, with the proviso that when Y is —OCO—, —NR¹⁰CO—, —OCH₂—, —SCH₂—, —SOCH₂—, —SO₂CH₂— or —NR¹⁰CH₂—,
   R⁵ is hydrogen, alkyl, alkoxy-alkyl-, alkenyl, alkynyl, benzyl, cycloalkyl-alkyl or phenyl-alkyl; and
   pharmaceutically active salts of carboxylic acids of formula I.
6. The method of any of statements 1-5, performed in vitro.
7. The method of statement 6, further comprising administering the one or more brown adipose cells to a mammal.
8. The method of any of statements 1-5, performed in vivo.
9. The method of statement 8, wherein the bexarotene, ciclopirox, IOX2, or combinations thereof is administered to a mammal in an amount sufficient to increase brown adipose tissue mass in the mammal relative to a control mammal that did not receive the bexarotene, ciclopirox, IOX2, or combinations thereof.
10. The method of statements 8 or 9, wherein the bexarotene, ciclopirox, IOX2, or combinations thereof is administered to a mammal for a time sufficient to increase brown adipose tissue mass in the mammal relative to a control mammal that did not receive the bexarotene, ciclopirox, IOX2, or combinations thereof.
11. The method of any of statements 8-10, wherein the bexarotene, ciclopirox, IOX2, or combinations thereof is administered once, twice, or three times per day.
12. The method of any of statements 8-10, wherein the bexarotene, ciclopirox, IOX2, or combinations thereof is administered daily, thrice weekly, biweekly, weekly, bimonthly, monthly, or a combination thereof.
13. The method of any of statements 8-10, wherein the mammal has lower body fat, has reduced white adipose tissue mass, consumes more oxygen, has increased energy expenditure, generates more heat, or any combination thereof, than a control mammal that did not receive the bexarotene, ciclopirox, IOX2, or combinations thereof.
14. The method of any of statements 8-13, wherein the mammal loses body weight within at least two weeks, or at least three weeks, or at least four weeks, or at least six weeks, or at least two months of receiving the bexarotene, ciclopirox, IOX2, or combinations thereof.
15. The method of any of statements 1-12, which does not comprise contacting the one or more starting cells with paulownin or an extract of Paulownia wood.
16. The method of any of statements 1-13, which does not comprise administering paulownin or an extract of Paulownia wood to a mammal.
17. A composition comprising bexarotene, bexarotene, ciclopirox, IOX2, or combinations thereof, and at least one supplemental ingredient selected from the group of retinoic acid, 9-cis retinoic acid, all-trans 3,4-didehydro retinoic acid, 4-oxo retinoic acid, retinol, rosigliotazone, forskolin, or any combination thereof.
18. The composition of statement 17, comprising at least two, or at least three, or at least four of the supplemental ingredients.
19. The composition of statement 17 or 18, further comprising one or more retinoids of formula I:

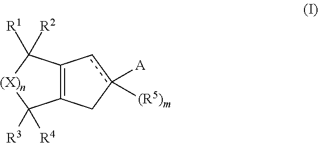

wherein:
   the dotted bond is either present and forms a double bond, or is absent;
   R¹, R², R³ and R⁴ are independently hydrogen or alkyl;
   n is 1, 2 or 3;
   X is —C(R⁸)(R⁹)— for n=1, 2 or 3; or X is oxygen for n=1;
   R⁸ and R⁹ are independently hydrogen or alkyl;
   R is hydrogen, alkyl, alkoxy, alkoxy-alkyl-, alkylthio, alkyl-NR¹⁰—, alkenyl, alkenyloxy, alkynyl, benzyl, cycloalkyl-alkyl, phenyl-alkyl, R¹⁰ is hydrogen or alkyl;
   m is 0 when the dotted bond is present; and m is 1 when the dotted bond is absent; and
   A is a residue of formula:

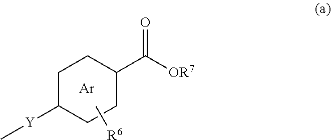

or of formula:

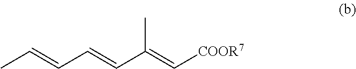

wherein
   Ar is phenyl or a heteroarylic ring;
   R⁶ is hydrogen, halogen, alkoxy or hydroxy;
   R⁷ is hydrogen or alkyl; and Y is —COO—, —OCO—, —CONR¹⁰—, —NR¹⁰CO—, —CH═CH—, —C≡C—, —COCH═CH—, —CHOHCH═CH—, —CH₂0-, —CH₂S—, —CH₂SO—, —CH₂S0₂-, —CH₂NR¹⁰—, —OCH₂—, —SCH₂—, —SOCH₂—, —SO₂CH₂— or —NR¹⁰CH₂—, with the proviso that when Y is —OCO—, —NR¹⁰CO—, —OCH₂—, —SCH₂—, —SOCH₂—, —SO₂CH₂— or —NR¹⁰CH₂—,
   R⁵ is hydrogen, alkyl, alkoxy-alkyl-, alkenyl, alkynyl, benzyl, cycloalkyl-alkyl or phenyl-alkyl; and
   pharmaceutically active salts of carboxylic acids of formula I.
20. A method comprising administering an effective amount of the composition of any of statements 17-19 to a mammal to thereby reduce body fat, reduce white adipose tissue mass, increase energy expenditure, generate more heat, and/or consume more oxygen.
21. The method of statement 20, wherein the effective amount of the composition is an amount sufficient to increase brown adipose tissue mass in the mammal relative to a control mammal that did not receive the composition.
22. The method of statement 20 or 21, wherein the composition is administered daily, thrice weekly, biweekly, weekly, bimonthly, monthly, or a combination thereof.
23. The method of any of statements 20-22, wherein the mammal loses body weight within at least two weeks, or at least three weeks, or at least four weeks, or at least six weeks, or at least two months of receiving the bexarotene, ciclopirox, IOX2, or combinations thereof.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a compound,” “a cell,” “a nucleic acid” or “a polypeptide” includes a plurality of such compounds, cells, nucleic acids or polypeptides (for example, a solution of cells, nucleic acids or polypeptides, a suspension of cells, or a series of compound, cell, nucleic acid or polypeptide preparations), and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

What is claimed:

1. A method of generating brown adipose cells from non-brown adipose starting cells, comprising contacting the starting cells with bexarotene, ciclopirox, IOX2, or combinations thereof, to thereby generate brown adipose cells.

2. The method of claim 1, wherein the starting cells are selected from the group of myoblasts, adipocytes, pre-adipocytes, mesenchymal precursor cells, multipotent stem cells, pluripotent stem cells, unipotent stem cells, fibroblasts, white adipocytes, and any combination thereof.

3. The method of claim 1, which inhibits white adipocyte cell generation.

4. The method of claim 1, further comprising contacting the one or more starting cells with retinoic acid, 9-cis retinoic acid, all-trans 3,4-didehydro retinoic acid, 4-oxo retinoic acid, retinol, rosigliotazone, forskolin, or any combination thereof.

5. The method of claim 1, further comprising contacting the one or more starting cells with one or more retinoids of formula I: 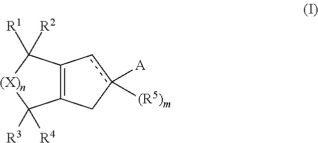
wherein:
the dotted bond is either present and forms a double bond, or is absent;
R¹, R², R³ and R⁴ are independently hydrogen or alkyl;
n is 1, 2 or 3;
X is —C(R⁸)(R⁹)— for n=1, 2 or 3; or X is oxygen for n=1;
R⁸ and R⁹ are independently hydrogen or alkyl;
R is hydrogen, alkyl, alkoxy, alkoxy-alkyl-, alkylthio, alkyl-NR¹⁰—, alkenyl, alkenyloxy, alkynyl, benzyl, cycloalkyl-alkyl, phenyl-alkyl, R¹⁰ is hydrogen or alkyl;
m is 0 when the dotted bond is present; and m is 1 when the dotted bond is absent; and
A is a residue of formula: 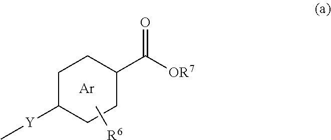
or of formula: 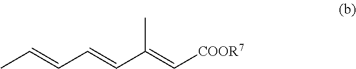
wherein
Ar is phenyl or a heteroarylic ring;
R⁶ is hydrogen, halogen, alkoxy or hydroxy;
R⁷ is hydrogen or alkyl; and Y is —COO—, —OCO—, —CONR¹⁰—, —NR¹⁰CO—, —CH═CH—, —C≡C—, —COCH═CH—, —CHOHCH═CH—, —CH₂0-, —CH₂S—, —CH₂SO—, —CH₂S0₂-, —CH₂NR¹⁰—, —OCH₂—, —SCH₂—, —SOCH₂—, —S0₂CH₂— or —NR¹⁰CH₂—, with the proviso that when Y is —OCO—, —NR¹⁰CO—, —OCH₂—, —SCH₂—, —SOCH₂—, —SO₂CH₂— or —NR¹⁰CH₂—,
R⁵ is hydrogen, alkyl, alkoxy-alkyl-, alkenyl, alkynyl, benzyl, cycloalkyl-alkyl or phenyl-alkyl; and
pharmaceutically active salts of carboxylic acids of formula I.

6. The method of claim 1, performed in vitro.

7. The method of claim 6, further comprising administering the one or more brown adipose cells to a mammal.

8. The method of claim 1, performed in vivo.

9. The method of claim 8, wherein the bexarotene, ciclopirox, IOX2, or combinations thereof is administered to a mammal in an amount sufficient to increase brown adipose tissue mass in the mammal relative to a control mammal that did not receive the bexarotene, ciclopirox, IOX2, or combinations thereof.

10. The method of claim 8, wherein the bexarotene, ciclopirox, IOX2, or combinations thereof is administered to a mammal for a time sufficient to increase brown adipose tissue mass in the mammal relative to a control mammal that did not receive the bexarotene, ciclopirox, IOX2, or combinations thereof.

11. The method of claim 8, wherein the bexarotene, ciclopirox, IOX2, or combinations thereof is administered once, twice, or three times per day.

12. The method of claim 8, wherein the bexarotene, ciclopirox, IOX2, or combinations thereof is administered daily, thrice weekly, biweekly, weekly, bimonthly, monthly, or a combination thereof.

13. The method of claim 8, wherein the mammal has lower body fat, has reduced white adipose tissue mass, consumes more oxygen, has increased energy expenditure, generates more heat, or any combination thereof, than a control mammal that did not receive the bexarotene, ciclopirox, IOX2, or combinations thereof.

14. The method of claim 8, wherein the mammal loses body weight within at least two weeks, or at least three weeks, or at least four weeks, or at least six weeks, or at least two months of receiving the bexarotene, ciclopirox, IOX2, or combinations thereof.

15. A composition comprising bexarotene, ciclopirox, IOX2, or combinations thereof, and at least one supplemental ingredient selected from the group of retinoic acid, 9-cis retinoic acid, all-trans 3,4-didehydro retinoic acid, 4-oxo retinoic acid, retinol, rosigliotazone, forskolin, or any combination thereof.

16. The composition of claim 15, comprising at least two, or at least three, or at least four of the supplemental ingredients.

17. The composition of claim 15, further comprising one or more retinoids of formula I: 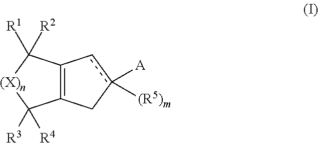
wherein:
the dotted bond is either present and forms a double bond, or is absent;
R¹, R², R³ and R⁴ are independently hydrogen or alkyl;
n is 1, 2 or 3;
X is —C(R⁸)(R⁹)— for n=1, 2 or 3; or X is oxygen for n=1;
R⁸ and R⁹ are independently hydrogen or alkyl;
R is hydrogen, alkyl, alkoxy, alkoxy-alkyl-, alkylthio, alkyl-NR¹⁰—, alkenyl, alkenyloxy, alkynyl, benzyl, cycloalkyl-alkyl, phenyl-alkyl, R¹⁰ is hydrogen or alkyl;
m is 0 when the dotted bond is present; and m is 1 when the dotted bond is absent; and
A is a residue of formula: 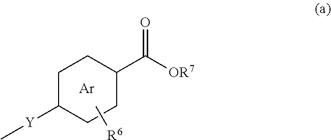
or of formula: 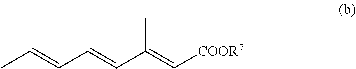
wherein
Ar is phenyl or a heteroarylic ring;
R⁶ is hydrogen, halogen, alkoxy or hydroxy;
R⁷ is hydrogen or alkyl; and Y is —COO—, —OCO—, —CONR¹⁰—, —NR¹⁰CO—, —CH═CH—, —C≡C—, —COCH═CH—, —CHOHCH═CH—, —CH₂0-, —CH₂S—, —CH₂SO—, —CH₂S0₂-, —CH₂NR¹⁰—, —OCH₂—, —SCH₂—, —SOCH₂—, —SO₂CH₂— or —NR¹⁰CH₂—, with the proviso that when Y is —OCO—, —NR¹⁰CO—, —OCH₂—, —SCH₂—, —SOCH₂—, —SO₂CH₂— or —NR¹⁰CH₂—,
R⁵ is hydrogen, alkyl, alkoxy-alkyl-, alkenyl, alkynyl, benzyl, cycloalkyl-alkyl or phenyl-alkyl; and
pharmaceutically active salts of carboxylic acids of formula I.
